(19) United States
(12) Patent Application Publication
(10) Pub. No.: US 2010/0312137 A1
(43) Pub. Date: Dec. 9, 2010

(54) Ovulation Cycle Monitoring and Management

(76) Inventors: Robert Gilmour, Auckland (NZ); Leonard Francis Blackwell, Manawatu (NZ)

Correspondence Address: DUANE MORRIS LLP - San Diego, 101 WEST BROADWAY, SUITE 900, SAN DIEGO, CA, 92101-8285, US

(21) Appl. No.: 12/084,166

(22) PCT Filed: Oct. 24, 2006
(86) PCT No.: PCT/IB2006/003925

§ 371 Date: Aug. 5, 2010

Related U.S. Application Data
(60) Provisional application No. 60/729,554, filed on Oct. 24, 2005.

(51) Int. Cl. A61B 10/00

(52) U.S. Cl. 600/551

(57) ABSTRACT

Methods of monitoring the ovulation cycle of an animal by detecting specific analytes in body fluids, computer program products, devices, data processing systems, and kits for monitoring the ovulation cycle and determining the fertility of female mammals.

### CROSS REFERENCE TO RELATED APPLICATIONS

This application claims priority from Provisional Application U.S. Ser. No. 60/729,554 filed Oct. 24, 2005, by Robert Gilmour and Len Blackwell, entitled “Ovulation Cycle Monitoring and Management”, the contents of which is hereby incorporated by reference in its entirety.

### FIELD

The field includes methods, devices, kits, and systems for monitoring, for example, mammalian ovulation cycles.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

The potentially fertile period of the ovulatory menstrual cycle, sometimes termed the window of fertility, is the period during which a female can conceive from an act of intercourse. In humans, this period begins up to six days before ovulation to allow for the fertilizable life of the sperm and ends one day after ovulation to allow for the fertilizable life of the ovum. Austin C R, J Reprod Fertil Suppl 22:75-89 (1975). Over 10% of couples in the United States have difficulty in achieving pregnancy. Chandra A., Fam Plann Perspect 30:34-42 (1998). Most of these couples require medical interventions. However, some may achieve pregnancy by having intercourse during the fertility window of the ovulatory cycle and timing it to the most fertile period of the cycle, and an accurate determination of the ovulatory cycle has many practical applications in the management of human fertility and infertility.

Monitoring and determination of the ovulatory cycle is also very important in fertility and reproductive management in animal husbandry. Considerable resources of the farm and domestic animal industries are dedicated to the reproductive and breeding managements of these animals. Economic considerations of the animal breeding business require owners to understand the reproductive cycle and how it can be managed and manipulated. In the dairy industry, for example, the percentage of cows that become pregnant during a breeding season has a direct effect on ranch profitability. In the equine industry, the periodicity of estrus and ovulation are linked to photoperiodic conditions, and management tools such as the use of artificial lighting and pharmaceutical treatments have been used to try to help breeders to gain a limited amount of control over a reproductive system that is often difficult to predict with an acceptable amount of certainty. While it is understood that detection, monitoring, and modulation of the animal estrous/ovulation cycles could increase the effectiveness of reproductive management, there remains a significant need for improvements in this area and the potential for improvement in the reproductive efficiency by maximizing heat detection and conception rates would be a major opportunity for these industries. The inventions described and claimed herein address this unmet need.

The ovulatory cycle has been the subject of much investigation. For example, the patterns of secretion of luteinizing hormone (LH), and of the ovarian hormones, estradiol and progesterone, have been investigated. Clinical studies have been reported concerning the measurement of these and other hormones in large population samples, including how the hormones may correlate with the fertility status of individual members of the population. One problem with these studies is that data obtained from large populations of females do not take into consideration the considerable variations from one individual to another, or the variation from one cycle to another in the same individual. For example, in a population of individual women reporting normal-length cycles (average 28 days), some individuals may exhibit extremely short cycle lengths. The whole cycle can be compressed into 20 or 21 days, or in extreme instances an even shorter interval. These shortened cycles may appear only occasionally, or more frequently. The fertile phase during these shortened cycles occurs quite early. It is thus apparent that one challenge to accurately monitoring fertility arises from the variability of the ovulation cycle amongst individuals and between cycles in particular individuals.

Data obtained from clinical studies is measured in a laboratory and interpreted by a physician or health care professional. To maintain accuracy and reliability standards, laboratories and clinics must be accredited and employ fully trained personnel for performing the assays, maintaining quality control and interpreting the results. Thus another impediment to use of ovulation monitoring assays is that most assays can only be performed currently by sophisticated laboratory instruments by persons so trained to use these instruments. This is inconvenient for the subject and costly.

A variety of immunoassay techniques and detection devices are available that allow analytes to be measured as biomarkers of physiological status. Included among the analytical systems used for detection of analytes are chromatographic assay systems. Such chromatographic systems are frequently used by physicians and medical technicians as point of care devices for in-office diagnosis. Chromatographic systems used in conjunction with immunoassays in a procedure known as immunochromatography allow use of a labeling reagent or particle that has been linked to an antibody for the molecule to be assayed, forming a conjugate. This conjugate is then mixed with a sample and, if the molecule to be assayed is present in the specimen, the labeling reagent-linked antibodies bind to the molecule to be assayed, thereby giving an indication that the molecule to be assayed is present. The labeling reagent or particle can be identifiable by color, magnetic properties, radioactivity, specific reactivity with another molecule, or another physical or chemical property. The specific reactions that are employed vary with the nature of the molecule being assayed and the sample to be tested.

Immunochromatographic assays may be classified generally into “sandwich” type assays and “competitive” assays, depending on the nature of the analyte-antibody complex to be detected and the steps needed to produce that complex. In the case of antigen detection, the sandwich immunochromatographic procedures mix a sample having a detectable analyte with antibodies to the analyte. The antibodies are typically mobile and linked to a label or a reagent, such as dyed latex, a colloidal metal sol, or a radioisotope. The mixture containing the antibody-analyte complex is separated by use of a chromatographic medium containing a capture zone. This capture zone contains immobilized antibodies for the analyte of interest. When the complex of the analyte and the labeled antibody reaches the zone of the immobilized antibodies on the chromatographic medium, binding occurs, and the bound-labeled antibodies are localized at the zone. This indicates the presence of the desired analyte. This technique can be used to obtain qualitative results. Examples of sandwich immunoassays performed on test strips are described in U.S. Pat. No. 4,168,146 to Grubb et al., U.S. Pat. No. 4,366,241 to Tom et al., U.S. Pat. Nos. 6,017,767 and 5,998,220 to Chandler; and U.S. Pat. No. 4,305,924 to Piasio et al. The use of other immunoassays, including lateral-flow assay systems and components in the field of molecular diagnostics, has also been described (see M. Surmanian, IVD Technology, October, 2004 and W. R. Seitz, “Immunoassay Labels Based on Chemiluminescence and Bioluminescence,” Clinical Biochemistry 17:120-126 (1984).

In a competitive immunoassay, the immobilized component is present in known amounts as a control, and the mobile component is present in unknown amounts. The unknown amount of mobile component is supplemented with a known amount of the same component that has been tagged by the addition of a measurable moiety which does not interfere with its immunochemical reactive properties. The tag may include, for example, a radioisotope, a chromophore, a particle, a fluorophor, or an enzyme. The amount of tagged material bound immuno-chemically to the solid phase depends upon the amount of untagged component in solution competing for the same binding sites. The amount of the unknown component present in inversely related the amount of bound tagged component.

In addition to immunochromatographic assays, enzyme-based chromatographic assays can be used. Similar techniques are used, except that an enzymatically-catalyzed reaction is used in place of an antigen-antibody reaction. The enzymatically-catalyzed reaction frequently generates a detectable product.

Representative examples of membrane based diagnostic tests, including lateral flow diagnostic tests, are known in the art. See U.S. Pat. No. 5,602,040 to May et al., and U.S. Pat. No. 5,075,078 to Osikowicz et al. For examples of lateral flow assay methods and apparatuses, where the reading is normally conducted optically, see U.S. Pat. Nos. 5,591,645 to Rosenstein; 5,798,273 to Shuler et al.; 5,622,871 to May et al; 5,602,040 to May et al.; 5,714,389 to Charlton et al.; 5,879,951 to Sy; 4,632,901 to Valkirs et al.; and 5,958,790 to Cerny. Examples of assays with several different pads or membranes, each with defined functions, such as, for example, receiving of sample, storing and releasing of conjugate, and carrying test and control lines for the presence of the analyte in the sample, are noted in U.S. Pat. Nos. 5,559,041, 5,728,587, and 6,027,943, all issued to Kang et al. Pads with carbon black immunochemical label or reporting molecule are referenced in U.S. Pat. No. 5,252,496 to Kang et al. Other representative examples of assays include lateral-flow dip-stick tests (U.S. Pat. No. 5,591,645 to Rosenstein) and flow through tests as set forth in for example, U.S. Pat. No. 5,395,754 Lambotte et al., U.S. Pat. No. 4,916,056 to Brown et al., and U.S. Pat. No. 5,149,622 to Brown et al.

Various solid phase testing devices such as dipsticks and chromatographic strips, which may readily be adapted for use in determining urinary analytes, are also known in the art. Representative examples of assays which can readily be adapted for use in accordance with the teachings of the present invention are described, for example, in U.S. Pat. No. 5,500,350 to Baker et al., U.S. Pat. No. 5,604,110 to Baker et al., U.S. Pat. No. 4,999,285 to Stiso, U.S. Pat. No. 4,861,711 to Friesen, U.S. Pat. No. 5,602,040 to May et al., U.S. Pat. No. 5,622,871 to May et al., U.S. Pat. No. 5,656,503 to May et al., U.S. Pat. No. 6,187,598 to May et al., U.S. Pat. No. 6,228,660 to May et al., U.S. Pat. No. 6,818,455 to May et al., US Pat. App. No. 2001041368 to May et al., US Pat. App. No. 2001008774 to May et al., U.S. Pat. No. 6,352,862 to Davis et al., US Pat. App. No. 2003143755 to Davis et al., US Pat. App. No. 2003207465 to Davis et al., and US Pat. App. No. 2003219908 to Davis et al.

In-home assays developed for monitoring ovulation include those based on use of sandwich assays to monitor urinary levels of luteinizing hormone (LH). LH levels peak approximately one day prior to ovulation, and the change in levels is generally large enough that measuring the LH change can be visualized by the human eye on a color coded standardized chart.

However, changes in analyte concentration are seldom so dramatic and thus necessitate the use of more sensitive instrumentation if the data are to be accurately measured. An assay that is not sufficiently accurate is prone to misinterpretation, particularly by a non-professional. See for example, Brown J B, et al., American Journal of Obstetrics & Gynecology. 157(4 Pt 2):1082-9, (1987). For example, it is possible that a woman using an in home assay with a simple eye test could misread the test results when others would interpret the test differently, particularly when the test indications disagreed with the woman's preconceptions of her ovarian activity. A more quantitative assay with an absolute read out would be desirable to prevent this misreading.

Attempts have been made to use an ovarian monitor for in home measurement of estrone glucuronide (E1G) and pregnanediol glucuronide (PdG). Blackwell L. F., et al., Steroids 68:465-476 (2003), incorporated by reference herein. In this study, the results from the Ovarian Monitor were compared to results obtained from radioimmunoassays. It was reported that in 50% of the cycles a urine bias in the ovarian monitor test caused a delay of up to 3 days in identifying the beginning of the E1G rise compared with the radioimmunoassay, which was reported as being more reliable. Id. at 469. The E1G values obtained using the monitor were higher than those obtained by RIA, and this was reported as being attributed to a vertical displacement of the profiles resulting from a bias caused by interfering substances in the relatively large volumes of urine and the prolonged incubation times required for obtaining the necessary sensitivity of the assay. Id, at 474.

There is a need for better in-home and on-site fertility status assays that rivals the accuracy of assays performed in clinical settings, and which are simple, convenient, and cost effective. The use of quantitative strips offers a more flexible system for on-site and home fertility care than Monitors such as described in Blackwell L. F., et al., Id, which although accurate suffer from the disadvantage of not having the ease of use provided by a quantitative strip system such as described herein. Such assays would provide considerable savings and enable accurate and cost-effective daily monitoring of ovarian activity. Additionally, a quantitative home assay kit with improved accuracy over other strip systems is needed. The inventions herein address these and other needs.

### BRIEF SUMMARY

The inventions described and claimed herein have many attributes and embodiments including, but not limited to, those set forth or described or referenced in this Summary and elsewhere. The inventions are not limited to or by the features or embodiments identified in this Summary, which is included for purposes of illustration only and not restriction.

Methods and devices for use in monitoring the ovulation cycle in female animals are included. The methods and devices provide information useful, for example, for measuring the fertility of a female animal and for providing fertility management.

In one aspect the fertility of a mammal (including a human) is measured or evaluated by detecting specific analytes in body fluids. Particular analytes detected by methods and devices provided herein include hormones, hormone derivatives, and hormone metabolites, such as estrogen metabolites and progesterone metabolites. Analytes can by detected by immunological procedures described herein or by methods now known or later discovered.

Suitable hormone metabolites useful in monitoring the ovulation cycle include urinary glucuronides. Particular hormone metabolites for detection include the estrone glucuronide (E1G), an estrogen metabolite, and pregnanediol glucuronide (PdG), a progesterone metabolite. Analytes can be detected by binding to binding agents that bind with desired affinity and specificity. Suitable binding agents include antibodies, for example, antibodies directed to estrone glucuronide and antibodies directed to pregnanediol glucuronide.

One embodiment is directed to a method of measuring the fertility of a mammal comprising the steps of (a) obtaining a body fluid sample from a female subject; (b) contacting the sample with a capture element having a first binding agent capable of binding an estrogen metabolite and a second binding agent capable of binding a progesterone metabolite; (c) quantifying the excretion rate of said estrogen metabolite and said progesterone metabolite; and (d) determining the ovulation cycle status of said female subject based upon the relative excretion rates of said estrogen metabolite and said progesterone metabolite. The relative excretion rates may optionally be expressed as a ratio.

In some embodiments, binding agents are immobilized to a solid phase capture element such as strips, membranes, and the like. In certain embodiments, estrone glucuronide and pregnanediol glucuronide are detected by immunoassay procedures, including but not limited to those described herein. In further certain embodiments, estrone glucuronide and pregnanediol glucuronide are quantified by immunoassay procedures.

One, two, or more analytes can be evaluated or measured in one assay using a single body fluid testing device, including devices capable of reading multiple assay strips, and alternatively by devices that are capable of reading a single strip for the detection of two or more different analytes. In one exemplary embodiment, a single strip comprising antibodies against estrone glucuronide and antibodies against pregnanediol glucuronide is provided. Detection of analytes can be accomplished, for example, by a simple positive or negative format based upon a predetermined threshold value. In other embodiments, the amount of the analyte is quantified. In certain embodiments, analyte excretion rates are determined by the use of a quantitative strip/device. In particular embodiments, a solid phase test strip is used in which the paramagnetic particles are embedded or immobilized to the strip.

In another aspect, an analyte detector is provided that is capable of detecting analytes of interest, such as estrone glucuronide and pregnanediol glucuronide, for example. In certain embodiments, the analyte detector is portable and is suitable for use in a home or field location. The detector, whether portable or not, may be in communication with an electronic database. Certain embodiments include a portable detector in communication with an electronic database comprising historical and other values of levels/excretion rates for one or more estrogen metabolites and/or one or more progesterone metabolites.

Some embodiments of the analyte detector utilize a lateral flow assay format. Certain embodiments of the analyte detector utilize paramagnetic or superparamagnetic particles for detecting analytes, including but not limited to estrone glucuronide and pregnanediol glucuronide.

In another aspect, a fertility monitoring system is provided. A monitoring system according to the invention may comprise a fertility monitor having a sample dispenser that provides a fixed sample volume to a test strip. Certain embodiments of a fertility monitor provided herein comprise a sample dispenser that dispenses an adjusted sample volume to a test strip.

In some embodiments, an algorithm is used for calculating an adjusted urinary volume. Embodiments of the fertility monitor may include one or more of a sensor for detecting the presence of analytes in the sample, a processor for performing calculations, and means for communication to an external database or an internal data storage database.

In some embodiments, the excretion rates of certain hormone metabolites from urine are determined and compared to a compilation of data for a particular analyte. The compilation of data may be in the form of an electronic database, and in certain embodiments a compilation of data is directed specifically to a particular species of animal, or to a particular individual, or a set or subset of individuals or groups of individuals. Certain databases relate to data for estrone glucuronide and pregnanediol glucuronide excretion rates determined under a variety of selected conditions. For example, in certain embodiments the excretion rates for estrone glucuronide and pregnanediol glucuronide for at least one bovine ovulation cycle are provided.

In certain embodiments, urine samples are collected over a specified interval(s) of time as part of a method for determining or providing excretion rates for particular analyte. In some embodiments, urine is collected over at least a 3 hour time period and the volume of the urine sample is measured, and then adjusted to a normalized volume that corresponds to the time interval of the collection period.

In certain embodiments, the volume of a urine sample is normalized prior to determining excretion rates for a metabolite, for example, an estrogen metabolite and/or a progesterone metabolite. In some embodiments, normalizing a volume comprises adjusting excretion rates using a computer algorithm for correction of urinary volume bias.

In another aspect, information obtained regarding ovulation cycle status is used to measure or quantify fertility in a female animal, including determining a time frame for optimal fertility within a menstrual cycle of a female subject.

Embodiments of the inventions described herein are useful for determining time frames for optimal fertility for performing an in vitro fertilization of a female subject.

Other embodiments provided herein are useful for monitoring and/or treatment of a female subject suffering from or suspected of having a post partum condition.

Embodiments of the inventions described herein are also useful for detecting and/or treating menopause and/or symptoms associated with menopause in a female subject (including e.g. natural menopause, perimenopause, induced menopause, premature menopause, and post menopause).

Embodiments of the inventions described herein are useful for administration of hormone replacement associated with menopause.

Embodiments of the inventions described herein are useful for the measurement of metabolites and/or analytes or the detection of cancers. In particular embodiments, hormone metabolites are monitored for the detection of certain cancers (e.g. estrogen levels for monitoring breast cancer).

Other conditions that are diagnosed and/or treated by embodiments of the invention include: anovulation associated with infertility, unexplained infertility, menopausal symptoms (perimenopausal menorrhagia, postmenopausal bleeding), premature menopause, amenorrhea, hormone imbalance (unspecified), decreased libido, chronic fatigue, nervousness, osteoporosis, premenstral syndrome, ovulation bleeding, dysfunctional uterine bleeding, hormone replacement therapy, surgical menopause syndrome, hypomenorrhea, hyperstimulated ovaries, polycystic ovarian disease, habitual aborter (currently pregnant again), missed abortion, and threatened abortion.

In another aspect, embodiments of the invention, are used in making detection devices (e.g. strips for the measurement of hormones) with long shelf life.

In certain embodiments, one or more hormone metabolites is/are measured for one or more days, or on a daily basis for a desired period of time or times. The provided algorithms may be used to determine or analyze excretion rates or to set one or more threshold value for analyzing analyte levels. In some embodiments, excretion rates for particular analytes are stored in a database that is in communication with an analyte detection device in communication with the database.

One method of determining analyte excretion rates provided herein comprises applying a urine volume adjustment. Certain embodiments utilize a correction for urine volume. In alternative embodiments, urine volume corrections are made by applying a algorithm to adjust values in the quantification of an analyte or determination of excretion rate of an analyte, for example. In some embodiments, a urine sample volume correction is made with reference to the specific gravity determination made for a sample, including where urine is collected from a subject with or without respect to a specified time period. In some embodiments, a urine sample volume correction is made based upon a spectroscopic analysis of a sample, including where urine is collected from a subject without respect to a specified time period

In another aspect, data processing systems are provided for use in performing methods of the invention. Certain embodiments are directed to methods of monitoring the physiologic status of one or more remotely located subjects in need of therapeutic management.

In one data processing system, a central data processing system is configured to communicate with and receive data from one or more subject monitoring systems. Each subject monitoring system is capable of one or more of receiving, storing, and/or analyzing subject data. An example of a method of monitoring a subject can be performed by the following steps: obtaining a sample from a subject for analysis; contacting the sample with an analyte detector associated with a subject monitoring system; measuring a photometric or electroactive signal corresponding to an analyte on the detection device and detecting one or more analyte; performing an exchange of data between said subject monitoring system and said central data processing system; generating a computer program product output comprising historical and/or real time physiologic status assessment data of said subject, wherein said computer program product output is in communication with the central data processing system; analyzing said subject data from one or more subject monitoring systems; determining the status of the subject based on the analysis performed by said computer program; and communicating, transmitting, or displaying the identified subject status and/or a therapeutic management recommendation for one or more subjects.

In certain embodiments, assays are performed on samples using a detection device suitable for a lateral flow assay system in conjunction with a subject monitoring system. A detection device for use in the subject monitor system may be capable of detecting photometric or electroactive signals generated from the specific analytes.

In some embodiments, subject data is transmitted from a subject monitoring system to, for example, a central data processing system to determine a subject's clinical and/or physiologic status. It is sometimes preferred that certain subject data transmitted from a subject monitoring system is analyzed substantially simultaneously with the transmission of the data to, for example, a central data processing system in order determine a subjects clinical or physiological status. In other aspects the determination of the subject's clinical or physiologic status includes ratiometric determination of body fluid excretion rates with or without volumetric adjustment for fluid volume bias using a computer executable algorithm.

In some embodiments, the method includes generation of a computer program product output (e.g., a database) comprising historical and real time physiologic status assessment data of the subject or other subjects in communication with, for example, a central data processing system.

In some embodiments, the method includes transmission and/or analysis of the subject data transmitted from one or more of the subject monitoring systems to, for example, a central database or data processing system and to, for example, the computer or other data receiving device of a physician or designated health care professional. In certain embodiments, the method includes transmission and/or analysis of the subject data transmitted from one or more of the subject monitoring systems to, for example, a central data storage and/or processing system substantially simultaneously with the transmission thereof to the computer or other data receiving device of a physician or designated health care professional.

In some embodiments, the method includes determining a subject's clinical or physiologic status based on an analysis performed by a computer program to identify the clinical and/or physiologic status of individual subjects. In certain other embodiments, a program evaluates potential abnormalities when compared against clinical or physiologic status assessment data from broader subject groups and/or populations.

In some embodiments, the method comprises one or more of communicating, transmitting, and/or displaying the identified subject clinical and/or physiologic status and therapeutic management recommendation for each respective subject via at least one remotely located client in communication with a central data storage/processing system and/or respective subject monitor system.

In some embodiments, the method comprises optimizing accuracy of a fertility status assessment and/or fertility status prediction of individual fertility endpoints based upon statistical comparison using individual historical data and or subject population historical data. In some embodiments, the method comprises transmitting information pertaining to the clinical and/or physiologic status of individual subjects when compared against clinical or physiologic status assessment data from broader subject groups or populations.

In some embodiments, the method comprises communicating, transmitting, and/or displaying the identified subject clinical and/or physiologic status and therapeutic management recommendation for each respective subject via at least one remotely located client in communication with a central data processing system and/or respective subject monitor system. In some embodiments, the method includes transmitting information pertaining to the clinical or physiologic status and clinical and/or physiologic issues of individual subjects including potential abnormalities when compared against clinical or physiologic status assessment data from broader subject groups and/or populations.

In a further embodiment, the method is performed by obtaining a sample from a subject for analysis and capturing the sample on detection device suitable for detection on or with a subject monitoring system. The detection device is assessed, and signals corresponding to one or more analytes are measured on or with the detection device. The subject data that is obtained is analyzed to determine a subject's clinical or physiologic status. The subject data is preferably analyzed within a short period of time, including by analyzing the subject data transmitted from a subject monitoring system substantially simultaneously with the transmission of the subject data to a central data processing system. A computer program product output (e.g., database) is generated that comprises historical and/or real time physiologic status assessment data of one or more subjects that are in communication with a central data storage or processing system. The subject data are transmitted from one or more subject monitoring systems to a central data storage or processing system and to the computer or other data receiving/viewing device of a physician or designated health care professional. Preferably, the subject data is transmitted from one or more subject monitoring systems to a central data storage or processing system and to the computer or other data receiving/viewing device of a physician or designated health care professional within a short period of time, such as substantially simultaneously. A subject's clinical or physiologic status is determined based on the analysis performed by the computer program in order to identify clinical or physiologic issues of individual subjects, including potential abnormalities when compared against clinical or physiologic status assessment data from broader subject populations. The subject's clinical or physiological status, including any detected abnormities, may be indicative of the subject's fertility. One or more subject's clinical or physiologic status is determined based on the analysis performed by the computer program, including to identify clinical or physiologic issues of individual subjects such as the identification of potential abnormalities when compared to clinical or physiologic status assessment data from broader subject populations. A subject's clinical or physiologic status may be determined based on performance of a data review or analysis. This may include the identification of clinical and/or physiologic issues of individual subjects, including the identification of potential abnormalities when compared to clinical or physiologic status assessment data from broader subject groups and/or populations. A subject's clinical and/or physiologic status is communicated, transmitted, and/or displayed. A therapeutic management recommendation can be made for one or more remotely located subject in communication with a central data storage or processing system and/or respective subject monitor system. The method is typically performed, for example, by using a central data processing system configured to communicate with and receive data from a one or more subject monitoring systems, where each subject monitoring system is capable of one or more of receiving, storing, and analyzing subject data.

In certain embodiments, the database contains data selected from the group consisting of physiologic data and behavioral data. In other embodiments, the database comprises historical and real-time physiologic status assessment data. In other embodiments, the database comprises historical and real time fertility status assessment data. In certain aspects, the database comprises data related to historical and real time urinary metabolite excretion rates. In certain other embodiments, the database comprises historical and real-time data directed to ratiometric measurements related to urinary metabolite excretion rates. In yet another aspect, the database comprises data related to historical and real time, for example, urinary glucuronide excretion rates. In yet another embodiment, the database contains physiologic data related to one or more of urinary metabolites, blood glucose measurements, body temperature measurements, presence or absence of illness, and assessments data related to diet, exercise, and stress. In certain embodiments, the database contains data directed to general health status, diet, exercise, and medications taken; date and time information of the last measurement; and prescribed course of action regimen(s). In some embodiments, the database of medication interaction information is configured to allow a subject to query the database for information related to the subject's use of multiple medications. In other embodiments, the database of medication interaction information is configured to allow a subject to query the database for specific historical fertility data profile for each subject and/or historical fertility profiles for groups and/or populations of subjects.

In one aspect, the algorithm assesses a subject's clinical and/or physiologic status based on comparison against clinical and/or physiologic status assessment data from broader subject populations, groups, and/or the subject. In other aspects, the algorithm calculates adjustments for a subject's ovulation variation according to a physician's or other health care professional's prescription as applied to the data entered into the system by the subject. In another aspect, the algorithm optimizes efficacy of the specific fertility regimen based on a particular subject's reproductive condition. In yet another aspect, the algorithm is configured to make automatic adjustments to a subject's self-monitoring and fertility management regimen based on subject-entered data.

In certain embodiments, the algorithm contains data useful for evaluation of the effects of concurrent therapy for other non-fertility indication which might affect the fertility or ovulation cycle of the subject.

In certain embodiments, the algorithm allows interactive input from a physician or other health care professional to specify retrospective and/or supplemental adjustment regimens.

In certain embodiments, the subject monitor system suitable for monitoring fertility management data of subjects is capable of detecting paramagnetic analyte signals.

In some embodiments, the system communication is performed by a device selected from the group comprising a transmitter, a beeper, a receiver, a telephone, a modem, a cellular phone, a cable, an internet connection, a world wide web link, a television, a closed circuit monitor, a computer, a display screen, a telephone answering machine, facsimile machine, or a printer.

A great advantage of the inventions provided herein are their great accuracy, high level of utility, and ease of use. Furthermore, certain embodiments of provided herein are very stable and have a long shelf life. This provides a very stable platform that is unaffected by unaffected by aging, heat or humidity, or other physical properties. The diagnostic agents (e.g. strips) can be read immediately, as well as days or months later to obtain a result. Thus in certain embodiments, it is envisioned that a woman can be traveling (e.g. camping, on a cruise ship, etc.) and do her tests, and then on return, all the tests can be read and the levels of E1G or PDG can be observed over the period she is away. This can be used to monitor the cycle, therapy for infertility or hormone replacement therapy by way of examples.

This new form of testing of the invention is novel and much need. The ease of use and this stability encourage compliance and provide much more convenient and efficient protocols than the current art. A further advantage is the ease of use is further enhanced when the handheld reader is used. This will provide people with the ability to perform self test whenever convenient such that the result can be received by a doctor and a doctors recommendation can be made remotely. Alternatively, for example, a recommendation may come from a computer, for example to advise a period of maximum fertility.

A great advantage of the inventions provided herein are their great accuracy, high level of utility, and ease of use. Furthermore, certain embodiments of provided herein are very stable and have a long shelf life. This provides a very stable platform that is unaffected by unaffected by aging, heat or humidity, or other physical properties. The diagnostic agents (e.g. strips) can be read immediately, as well as days or months later to obtain a result. Thus in certain embodiments, it is envisioned that a woman can be traveling (e.g. camping, on a cruise ship, etc.) and do her tests, and then on return, all the tests can be read and the levels of E1G or PDG can be observed over the period she is away. This can be used to monitor the cycle, therapy for infertility or hormone replacement therapy by way of examples.

This new form of testing of the invention is novel and much need. The ease of use and this stability encourage compliance and provide much more convenient and efficient protocols than the current art. A further advantage is the ease of use is further enhanced when the handheld version is used. This will provide people with the ability to perform self test whenever convenient such that the result can be received by a doctor and a doctors recommendation can be made remotely. Alternatively, for example, a recommendation may come from a computer, for example to advise a period of maximum fertility.

These and other aspects and embodiments of the inventions described and claimed herein will be apparent from and throughout the application and claims, all of which shall be considered to be a part of the written description thereof.

### BRIEF DESCRIPTION OF THE DRAWINGS

FIG. 1 is a schematic illustration of a fertility management system for human application.

FIG. 2 is a schematic illustration of a fertility management system for non-human application.

FIG. 3 shows an example of RIA data used for identifying the first rise in E1G using a modified Trigg's tracking signal algorithm. The tracking signal was calculated for each day of the cycle from the beginning of the cycle (first day) so that the algorithm is truly prospective. No baseline calculation is necessary.

FIG. 4 shows a standard curve for measurement of E1G in human urine samples utilizing strips which have been sprayed with an E1G-ovalbumin conjugate.

FIG. 5 shows a standard curve for measurement of PdG in human urine samples utilizing strips which have been sprayed with PdG-BSA as the capture material.

FIG. 6 shows menstrual cycle profiles for E1G and PdG based on urinary hormone excretion rates as measured by the color intensity on the strips. The PdG data were only collected once the E1G peak was detected.

FIGS. 7A and 7B show standard curves for measurement of E1G and PdG in dairy cow as measured in urine samples. The E1G and PdG data were obtained with ELISA assays.

FIG. 8 shows the daily E1G and PdG excretion rate profiles from cow 68.

FIG. 9 shows the daily E1G and PdG excretion rate profiles from cow 68 based on two consecutive cycles. These cycles are all corrected for variations in urine volume by use of creatinine excretion.

FIG. 10 shows the PdG concentration profile for an individual cow (cow 68) before a adjustment for urine volume is made according to creatinine profile.

FIG. 11 shows the close correlation between the bulling behavior of the animal and the ratio of E1G/PdG in order to adjust for the variations in the urine volume.

FIG. 12 shows a profile of pregnanediol glucuronide concentration as measured in milk (cow 68). The profile was similar to that from the urinary data, however, the PG level from the milk samples was much lower and no correction was made for variations in milk volume.

FIG. 13 shows the smoothing effect on the PdG concentration profile by normalization based on creatinine measurement (Jaffe reaction).

FIG. 14 shows the smoothing effect on the PdG excretion rate profile obtained with lateral flow strips by normalization based on specific gravity correction.

FIG. 15 shows determination of pregnancy via the use of PdG measurements utilizing ELISA assay for PdG with creatinine correction.

FIG. 16 shows similarity in the excretion rate profiles for E1G and PdG between measurements obtained by the half strips method and the measurements obtained by the Ovarian Monitor method for the same urine samples.

FIG. 17 illustrates an E1G MAR Standard Curve.

FIG. 18 shows a normalised menstrual cycle E1G excretion rates as measured by the MAR system and the ovarian monitor.

FIG. 19 illustrates a PdG MAR standard curve.

FIG. 20 shows a normalised menstrual cycle PdG excretion rates as measured by the MAR system and the ovarian monitor.

FIG. 21 illustrates the first rise day to estimated day of ovulation.

FIG. 22 shows days from E1G peak to PdG cut-off day.

FIG. 23 depicts factors influencing the PdG MAR standard curve correction methods

FIG. 24 illustrates urinary excretion of PdG without correction for urine volume in the cycling cow

FIG. 25 illustrates urinary excretion of PdG with correction for urine volume in the cycling cow.

FIG. 26 shows the urinary excretion of E1G and PdG with correction for urine volume in the cycling cow.

FIG. 27 illustrates the ratio of urinary excretion of E1G/PdG in the cow for the detection of estrus.

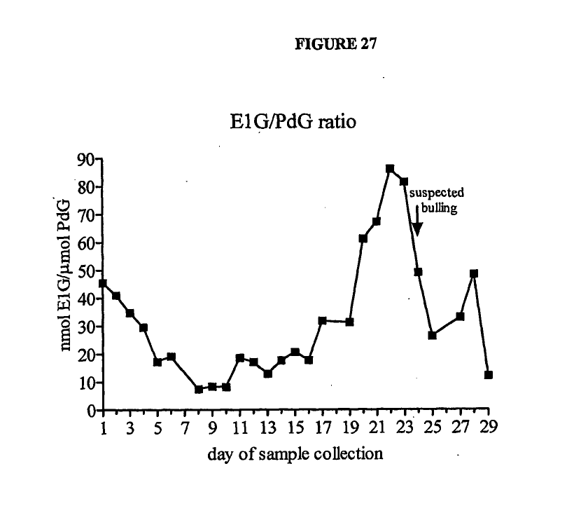

### DETAILED DESCRIPTION

The practice of the present invention may employ various conventional techniques of molecular biology (including recombinant techniques), microbiology, cell biology, biochemistry, nucleic acid chemistry, and immunology, which are within the skill of the art. Such techniques are explained fully in the literature, and include but are not limited to, by way of example only, MOLECULAR CLONING: A LABORATORY MANUAL., second edition (Sambrook et al., 1989) and MOLECULAR CLONING: A LABORATORY MANUAL., third edition (Sambrook and Russel, 2001), jointly and individually referred to herein as “Sambrook”; OLIGONUCLEOTIDE SYNTHESIS (M. J. Gait, ed., 1984); ANIMAL CELL CULTURE (R. I. Freshney, ed., 1987); HANDBOOK OF EXPERIMENTAL IMMUNOLOGY (D. M. Weir & C. C. Blackwell, eds.); GENE TRANSFER VECTORS FOR MAMMALIAN CELLS (J. M. Miller & M. P. Calos, eds., 1987); CURRENT PROTOCOLS IN MOLECULAR BIOLOGY (F. M. Ausubel et al., eds., 1987, including supplements through 2001); PCR: THE POLYMERASE CHAIN REACTION, (Mullis et al., eds., 1994); CURRENT PROTOCOLS IN IMMUNOLOGY (J. E. Coligan et al., eds., 1991); THE IMMUNOASSAY HANDBOOK (D. Wild, ed., Stockton Press NY, 1994); BIOCONJUGATE TECHNIQUES (Greg T. Hermanson, ed., Academic Press, 1996); METHODS OF IMMUNOLOGICAL ANALYSIS (R. Masseyeff, W. H. Albert, and N. A. Staines, eds., Weinheim: VCH Verlags gesellschaft mbH, 1993), Harlow and Lane (1988) ANTIBODIES, A LABORATORY MANUAL., Cold Spring Harbor Publications, New York, and Harlow and Lane (1999) USING ANTIBODIES: A LABORATORY MANUAL Cold Spring Harbor Laboratory Press, Cold Spring Harbor, N.Y. (jointly and individually referred to herein as Harlow and Lane), Beaucage et al. eds., CURRENT PROTOCOLS IN NUCLEIC ACID CHEMISTRY John Wiley & Sons, Inc., New York, 2000); and Agrawal., ed., PROTOCOLS FOR OLIGONUCLEOTIDES AND ANALOGS, SYNTHESIS AND PROPERTIES Humana Press Inc., New Jersey, 1993).

Unless indicated otherwise, the following terms have the following meanings when used herein and in the appended claims. Those terms that are not defined below or elsewhere in the specification shall have their art-recognized meaning.

“Analyte,” as used herein, is the substance to be detected which may be present in a test sample. The analyte can be any substance for which there exists a naturally occurring specific binding member (such as, an antibody), or for which a specific binding member can be prepared. Thus, an analyte is a substance that can bind to one or more specific binding members in an assay. “Analyte” also includes any antigenic substances, haptens, antibodies, and combinations thereof. As a member of a specific binding pair, the analyte can be detected by means of naturally occurring specific binding partners (pairs) such as the use of a lectin as a member of a specific binding pair for the determination of a carbohydrate. Analytes include proteins, peptides, amino acids, hormones, steroids, vitamins, drugs (including those administered for therapeutic purposes as well as illicit purposes), bacteria, viruses, and metabolites of or antibodies to any of the above substances. The details for the preparation of such antibodies and the suitability for use as specific binding members are well known to those skilled in the art. To those skilled in the art, it will also be appreciated that the body fluid “concentration” of the chosen analyte or analytes need not be measured in absolute terms. The analyte concentration may be measured in relative terms, e.g., as a range or a ratio relative to the concentration of a reference analyte present in the same sample of body fluid. Generally, it will be sufficient to assay an analyte in a manner which yields a signal, convertible to numerical data, related to the actual concentration, so that such data can be compared with similar data obtained at a different stage in the cycle to determine whether or not a significant change in actual concentration has occurred. Accordingly, where the specification and claims below refer to the “concentration” or “measurement” of an analyte, this expression is to be understood broadly.

Herein, the following abbreviations may be used for the following amino acids (and residues thereof): alanine (Ala, A); arginine (Arg, R); asparagine (Asn, N); aspartic acid (Asp, D); cysteine (Cys, C); glycine (Gly, G); glutamic acid (Glu, E); glutamine (Gln, Q); histidine (His, H); isoleucine (Ile, I); leucine (Leu, L); lysine (Lys, K); methionine (Met, M); phenylalanine (Phe, F); proline (Pro, P); serine (Ser, S); threonine (Thr, T); tryptophan (Trp, W); tyrosine (Tyr, Y); and valine (Val., V).

The term “amino acid sequence” refers to an oligopeptide, peptide, polypeptide, or protein sequence, a fragment of any of these, and to naturally occurring or synthetic molecules, as well as to electronic or other representations of foregoing suitable for use in conjunction with a computer, for example.

As used herein, analyte signals which are photometric include signals characterized by transmission of spectral wavelength detectable both visually and non-visually by methods and means recognized in the art, including for example, visible light, fluorescence, and phosphorescence.

As used herein, analyte signals which are electroactive included signals which are characterized by the generation of electric and magnetic fields detectable by art-recognized methods and means for detecting electric and magnetic fields. Representative analyte signals include, for example, signals from paramagnetic particles and/or supermagnetic particles in a Magnetic field.

The term “antibody” is used in the broadest sense, and includes monoclonal antibodies (including full length monoclonal antibodies, and agonist and antagonist antibodies), polyclonal antibodies, multispecific antibodies (e.g., bispecific antibodies), antibody fragments (e.g., Fab, F(ab)₂ and Fv), and antibody derivatives (e.g., recombinant or synthetic) so long as they exhibit a desired biological activity. These antibodies, binding portions or fragments thereof, hinge portions or fragments thereof, and effector regions or portions thereof, are all useful in the constructs of the invention.

The term “antibody fragment” refers to a portion of a full-length antibody, and includes the antigen binding or variable regions. Examples of antibody fragments include Fab, Fab′, F(ab′)₂ and Fv fragments. Papain digestion of antibodies produces two identical antigen binding fragments, called the Fab fragment, each with a single antigen binding site, and a residual Fc fragment. Pepsin treatment yields an F(ab′)₂ fragment that has two antigen binding fragments which are capable of cross-linking antigen, and a residual other fragment (which is termed pFc′). As used herein, “binding fragment” with respect to antibodies, refers to Fv, F(ab) and F(ab′)₂ fragments and functional mutants and analogs thereof. The Fab fragment, also designated as F(ab)′, also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region Fab′-SH is the designation herein for Fab′ in which the cysteine residue(s) of the constant domains have a free thiol group. F(ab′) fragments are produced by cleavage of the disulfide bond at the hinge cysteines of the F(ab′)₂ pepsin digestion product. Additional chemical couplings of antibody fragments are known to those of ordinary skill in the art.

“Binding proteins” include antibodies, monoclonal antibodies, antibody fragments (including Fab, Fab′, F(ab′)₂, and Fv fragments), linear antibodies, single-chain antibody molecules, multispecific antibodies formed from antibody fragments, or other antigen-binding proteins, any of which may be chimeric, humanized, or otherwise altered to be less immunogenic in a subject.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies may be made, for example, by the hybridoma method first described by Kohler and Milstein, Nature 256:495 (1975), or may be made by recombinant methods, e.g., as described in the art. Monoclonal antibodies may also be isolated from phage antibody libraries using the techniques described in Clackson et al., Nature 352:624-628 (1991), as well as in Marks et al., J. Mol. Biol. 222:581-597 (1991).

In general, the term “biologically active” refers to a molecule having a specified function or functions. The functional activity or activities may be less than, greater than, or about the same as, a naturally occurring molecule.

As used herein, the term “derivative” includes a chemical modification of a polypeptide, polynucleotide, or other molecule. In the context of this invention, a “derivative polypeptide”, for example, one modified by glycosylation, pegylation, or any similar process, retains at least one activity. For example, the term “derivative” of binding protein includes binding proteins, variants, or fragments that have been chemically modified, as, for example, by addition of one or more polyethylene glycol molecules, sugars, phosphates, and/or other such molecules. Polypeptides may also be “derived” from a reference polypeptide by having, for example, amino acid substitutions, deletions, or insertions relative to a reference polypeptide. Thus, a polypeptide may be “derived” from a wild-type polypeptide or from any other polypeptide. As used herein, a compound, including polypeptides, may also be “derived” from a particular source, for example from a particular organism, tissue type, or from a particular polypeptide, nucleic acid, or other compound that is present in a particular organism or a particular tissue type.

As used herein, the expression “fertile phase” is used to mean that interval in a female menstrual cycle, spanning the event of ovulation, during which it is possible that intercourse will result in fertilization, because of the normal viability of spermatozoa and ova.

The term “high affinity” for binding proteins described herein refers to an association constant (Ka) of at least about 10⁶M⁻¹ or 10⁷M⁻¹, preferably at least about 10⁸M⁻¹, more preferably at least about 10⁹M⁻¹ or greater, more preferably at least about 10¹²M⁻¹ or greater, for example, up to 10¹²M⁻¹ or greater.

“Indicator reagents” may be used in various assay formats useful in the inventions, including those identified or described herein. The “indicator reagent” comprises a “signal generating compound” (label) which is capable of generating a measurable signal detectable by external means conjugated (attached) to a specific binding member for the analyte. “Specific binding member” as used herein means a member of a specific binding pair. That is, two different molecules where one of the molecules through chemical or physical means specifically binds to the second molecule. In addition to being an antibody member of a specific binding pair for the analyte, the indicator reagent also can be a member of any specific binding pair, including either hapten-anti-hapten systems such as biotin or anti-biotin, avidin, streptavidin, or biotin, a carbohydrate or a lectin, a complementary nucleotide sequence, an effector or a receptor molecule, an enzyme cofactor and an enzyme, an enzyme inhibitor or an enzyme, and the like. An immunoreactive specific binding member can be an antibody, an antigen, or an antibody/antigen complex that is capable of binding either to the analyte as in a sandwich assay, to the capture reagent as in a competitive assay, or to the ancillary specific binding member as in an indirect assay.

As used herein, the term “Internet” incorporates the term “computer network” such as an “Intranet,” and any references to accessing the Internet shall be understood to mean 25, accessing a hardwired computer network as well. Herein, the term “computer network” shall incorporate publicly accessible computer networks and private computer networks, and shall be understood to support modem dial-up connections.

An “isolated” molecule (for example, a polypeptide or polynucleotide) refers to a molecule that is present outside of from its original environment or has been removed from its original environment (for example, the natural environment if it is naturally-occurring). For example, a naturally-occurring polynucleotide or polypeptide present in a living animal is not isolated, but the same polynucleotide or polypeptide, separated from some or all of the coexisting materials in the natural system (for example, proteins, lipids, carbohydrates, nucleic acids), is isolated.

The term “ligand” as used in the present invention refers to antigens, antibodies, haptens, hormones and their receptors, deoxyribonucleic acid and other organic substances for which a specific-binding material can be provided.

“Mammal” for purposes of treatment refers to any animal classified as a mammal, including human, domestic and farm animals, nonhuman primates, and zoo, sports, or pet animals, such as dogs, horses, cats, cows, etc.

The term “sample” includes biological samples which can be tested by the methods of the present invention described herein and include human and animal body fluids such as crevicular fluid, sweat, sebum, tears, vaginal fluid, whole blood, serum, plasma, cerebrospinal fluid, urine, lymph fluids, and various external secretions of the respiratory, intestinal and genitourinary tracts, tears, saliva, milk, white blood cells, myelomas and the like, biological fluids such as cell culture supernatants, fixed tissue specimens and fixed cell specimens. Any substance which can be diluted and tested using, for example, assay formats described or identified herein are contemplated to be within the scope of the present invention.

The various “signal generating compounds” (labels) contemplated include chromogens, catalysts such as enzymes, luminescent compounds such as fluorescein and rhodamine, chemiluminescent compounds, radioactive elements, and direct visual labels. Examples of enzymes include alkaline phosphatase, horseradish peroxidase, beta-galactosidase, and the like. The selection of a particular label is not critical, but it will be capable of producing a signal either by itself or in conjunction with one or more additional substances. The labels can also be visible label, for example, colloidal gold, colored latex particle, or an invisible label, for example, paramagnetic particles (PMPs), including superparamagnetic particles, or other PMPs which have surface properties that allow antibodies or recognition labels to be conjugated to the particles.

The term “therapeutically effective amount” means the amount of the subject compound that will elicit a desired response, for example, in a tissue, system, animal, or human that is sought, for example, by a researcher, veterinarian, medical doctor, or clinician. “Treatment” refers to both therapeutic treatment and prophylactic or preventative measures. Those in need of treatment include those already with the condition as well as those in which the condition is to be prevented or facilitated or its progress stopped or slowed or monitored.

In one aspect, the invention is directed to monitoring the ovulation cycle in female animals (e.g., mammals). As will be appreciated by one of skill in the art, the invention may be embodied as a method, a computer program product, a device, a data processing system, or as a kit. Information provided from various aspects of the invention is useful, for example, for measuring the fertility of the female mammal, which in turn is useful for enhancing fertility or for contraception. One aspect of the invention includes measuring the fertility of a mammal (including humans) by detecting specific analytes in body fluids.

Various body fluids may be tested, including for example blood, crevicular fluid, fecal material, milk, mucus, sweat, sebum, tears, urine, saliva, and vaginal fluid. Certain body fluids might be preferred for a particular animal species, and more than one type of body fluid can be analyzed. The methods, kits and devices provided herein can be conveniently used by persons that are not trained in performing medical testing procedures. In some embodiments, they are portable so they can be used at home or in an environment where certain animals are located. Where the body fluid is from a human subject, the sample can be taken by the subject herself or by another person. Alternatively, a sample is taken without direct human participation, for example, as part of an automated collection device or process.

The analyte concentration may be measured in absolute terms, or in relative terms such as a ratio relative to the concentration of a reference analyte present in the same sample of body fluid. Analytes can by detected, for example, by immunological procedures known in the art. Analytes of interest in the invention include, for example, hormones, hormone derivatives, and hormone metabolites such as estrogen metabolites and progesterone metabolites (e.g. those indicative of fertility).

Examples of estrogen metabolites that may be detected include, for example, estrone 3-sulfate, 2-hydroxyestrone, 4-hydroxyestrone, 2-methoxyestrone, 4-methoxyestrone, 2-methoxyestrone 3-sulfate, 2-methoxyestrone 3-glucuronide, 16 alpha-hydroxyestrone, estradiol-17α, estradiol 17β, 16-glucuronide-estriol; estradiol-17beta 3-glucuronide; estradiol-17beta 3-sulfate, 2-hydroxy-estradiol-17β, 2-methoxy-estradiol-17β, 2-methoxyestradiol-17beta 3-sulfate, 2-methoxy-estradiol-17beta 3-glucuronide, 6β-hydroxy-estradiol-17β, 2-methoxyestradiol, 17-epiestriol, 2-hydroxyestradiol, 16-ketoestradiol, 16β-hydroestrone, 16-epiestriol. In certain embodiments, estrogen and metabolites thereof include, for example, estradiol, estrone, estriol, 2(OH) Estrone, 4 hydroxy-estrone, 16α-hydroxy-estrone, 2-methoxyestrone, and 4-methoxyestrone. A particularly suitable estrogen metabolite for detection is estrone glucuronide.

Analytes of interest for certain embodiments include progesterone and progesterone metabolites. Major urinary metabolite of progesterone include, for example, 5β-pregnan-3α, 20α-diol glucuronide. Plasma metabolite of progesterone include, for example, 5β-pregnan-3α-ol-20-1-(5β-pregnenolone) and 5α-pregnan-3α-ol-20-1-(5α-pregnenolone). A particularly suitable progesterone metabolite for detection is pregnanediol glucuronide (PdG).

Binding Agents

An analyte of interest is capable of binding with a desired affinity to a binding agent described herein. Suitable binding agents include antibodies or fragments thereof, ligand and binding agent pairs, receptors, and the like. Certain embodiments have a first binding element comprising antibodies or fragments thereof capable of binding estrone glucuronide and a second binding element comprising antibodies or fragments thereof capable of binding pregnanediol glucuronide. Estrone glucuronide and pregnanediol glucuronide may, for example, be detected by the use of polyclonal antibodies or monoclonal antibodies that serve as a binding agent.

Suitable antibodies for detection of estrogen metabolites include, for example, mouse anti-estrone 3 glucuronide monoclonal antibody (unconjugated, Clone M7021931 from Fitzgerald Industries International); mouse anti-estrone sulphate (ES) monoclonal antibody (unconjugated, Clone M56261, from Fitzgerald Industries International); and anti-estrone 3 glucuronide monoclonal antibody (unconjugated, clone 9.F.25 from United States Biological., Swampscott, Mass. 01907). Representative antibodies useful for detection of progesterone metabolite include, for example, anti-Pregnandiol-3-alpha-Glucuronide Monoclonal Antibody, unconjugated, Clone 8.F.233 (United States Biological; Swampscott, Mass. 01907).

Particularly suitable antibodies against E1G have been described in the literature. See, for example, Lewis J O, et al., Steroids 59 (4) 288-191 (1994), and Henderson K. M., et al., Clin Chim Acta. December 29; 243(2):191-203 (1995), each of which is incorporated by reference herein. Particularly suitable antibodies against PdG are commercially available (East Coast Biologicals, North Berwick, Me.).

### Capture Elements

The invention may employ various functional means for immobilizing or capturing analytes (e.g. hormones and hormone metabolites), including those described herein or known in the art. Binding agents may be immobilized or otherwise attached to one or more capture elements. A capture element is preferably associated with a solid phase, but liquid phase capture elements may also be used. Suitable capture elements include porous materials, such as glass fiber, membranes, papers, strips, pads, and the like. Suitable membranes include nylon, nitrocellulose, polyester material, and the like.

In certain embodiments, test strips and kits are provided that are particularly useful for monitoring the ovulation cycle of a female animal. One, two, or more analytes may be captured on a single strip, such as a single lateral flow strip. For example, in some embodiments, more than one antibody is immobilized to a single capture element such that a single capture element (e.g. a strip) is capable of detecting one or more analytes. Antibodies or other binding agents may be conjugated to or associated with a detection element. In one embodiment, a single strip comprising antibodies against estrone glucuronide and antibodies against pregnanediol glucuronide is provided.

One or more analytes can be measured in a single assay, including an assay performed using a single body fluid testing device that is capable of performing assays on more than one strip (e.g. two or more strips), or alternatively, a device that detects two or more analytes independently on a single strip. One embodiment is directed to a single quantitative test strip for detecting and quantifying estrone glucuronide and pregnanediol glucuronide. In embodiments where more than one different analyte is detected in the same sample liquid, it is desirable to have reaction conditions balanced to maximize efficiency for the detection of each analyte.

In one embodiment, a strip assay for a first analyte uses a labeled particle for detection that comprises an antibody specific for the first analyte. The strip has a detection zone that comprises immobilized analyte or an analogue thereof. Each labeled particle may comprise a plurality of identical antibody molecules. The amount of biologically active antibody for a particular analyte on each particle can be standardized. The concentration of analyte or analyte analogue in the detection zone should be in excess of the effective concentration (molar concentration) of antibody on the particles. The quantity of particle-labeled antibody available in the assay should be in excess, relative to the anticipated analyte concentration in the sample. These levels can be adjusted so that the presence of free analyte in the sample results in a significant level of binding of the free analyte to the antibodies on the particles and thus inhibits binding of the particle label to the immobilized analyte/analogue in the detection zone. It is sometimes desirable for the average particle to have a sufficient number of active antibody molecules to ensure binding of the particle in the detection zone, but in an amount where the presence of analyte in the sample has a limiting effect on this binding. In this embodiment, the extent to which the particles become bound in the detection zone is therefore inversely proportional to the concentration of analyte in the sample liquid.

In certain embodiments of a strip assay, the particle labeled antibody is placed upstream from the detection zone so that a liquid sample contacts the particle labeled material and carries it to the detection zone. In this assay, it is preferred that the potential reaction between the free analyte and the particle-labeled antibody is at least substantially complete before these reagents reach the detection zone. In this assay, the extent to which the particles bind to the immobilized analyte/analogue in the detection zone is a function of the residual uncomplexed antibody remaining on the particles. Thus, the concentration of immobilized analyte/analogue in the detection zone should be high in order to promote efficient capture of the particles as they pass through this zone. It is also desirable that the antibody on the particles has a high affinity for the analyte to enhance the efficiency of the previous binding of the particle-labeled antibody to free analyte in the sample liquid. This affinity is typically at least about 10⁸, preferably at least about 10⁹, and more preferably at least about 10¹⁰, litres/mole.

Antibodies or antigens can be dispensed onto the test and control lines on the membrane and may be coupled with anchor proteins (e.g., avidin, streptavidin, biotin). The membrane may be blocked using blocking buffers that contain bulk proteins, (e.g., casein, bovine serum albumin) and treated with surfactants for its long-term stability and flow characteristics. Certain reagents may be added to the striping solutions to ensure more-consistent dispensing and binding, and prevent hydrophilicity at the test and control lines (Tween 20 is used at very low concentrations).

In certain embodiments, low concentrations of alcohols may be used to precipitate proteins onto the membranes to assist in binding. Surfactants may be used to make the pad hydrophilic, particularly if it is a glass fiber or polyester pad. In certain embodiments, polymers may be added to harden the pad and control the flow rate. In certain embodiments, antibodies or other biochemical reagents may be added to capture red blood cells or mucins. In further embodiments, buffering components may be used such that a sample will be at a desired pH when it reaches the conjugate pad.

Chemical and biological treatments can be performed on a sample at various times, before it contacts a capture element. Such treatments may include, for example, removing red blood cell, or removing mucins or other interfering components from a sample before it reaches a capture element. In certain embodiments, a sample is pretreated to facilitate the availability of antigenic sites are available for the assay, or removes interfering components before adding a sample.

### Analyte Detection

The invention may employ various functional means for detection analytes (e.g. hormones and hormone metabolites), including those described herein or known in the art. Detection reagents may form complexes with an analyte or binding element to allow an analyte to be detected. These may include complexes between analyte-specific binding molecules (for example, antibodies) and different possible reporter molecules such as, for example, enzymes (e.g. horseradish peroxidase), dyes, radionuclides, luminescent groups, fluorescent groups, biotin, colliodal particles (see U.S. Pat. No. 7,122,196 to Reed et al., and U.S. Pat. No. 6,586,193 to Yguerabide et al.), metal colloids such as colloidal gold and selenium, non-metal colloids, nanoparticles, polymeric beads and latex beads, carbon black label (U.S. Pat. No. 5,252,496 to Kang et al) and metal sol reagents and conjugates (U.S. Pat. No. 5,514,602 to Brooks, Jr. et al), as well as the use of liposomes mediated carrier dye molecules, and non-visual reporting molecules or labels such as, for example, paramagnetic particles. The detection element (e.g. conjugate) may be a biological component (e.g., antibody, antigen, hapten) that is bonded to a visible label (e.g., colloidal gold, colored latex particle), or an invisible label (e.g., paramagnetic particle). The conjugation of a binding agent to reporter group may be achieved using standard methods known to those of ordinary skill in the art and may also be purchased conjugated to a variety of reporter groups from many commercial sources (e.g., Zymed Laboratories, San Francisco, Calif., and Pierce, Rockford, Ill.).

Detection of analytes can be accomplished by standard assay techniques known in the art. Analytes can be detected in a simple positive or negative format based upon a predetermined threshold value. In some embodiments, it is preferred that the amount of the analyte is quantified. This may be, for example, an absolute quantification or a excretion rate quantification. Analytes can be quantified by measuring the band intensity on a strip that corresponds to that analyte. In some embodiments, more than one analyte is detected or quantified in a multiplexed assay. In another aspect, analyte excretion rates are determined by the use of a quantitative strip in certain embodiments. In other embodiments, an antibody-particle (e.g. nanoparticle) conjugate is not preformed, but rather the attachment or immobilization takes place upon hydration of an antibody or binding agent together with a nanoparticle (see WO 2005/051295A2 to Lin, R. et al., entitled “Asymmetrically Branched Polymer Conjugates and Microarray Assays”, incorporated by reference herein).

In certain embodiments a capture element such as pads, membranes, test strips, and the like are used in conjunction with paramagnetic particle mediated detection. The paramagnetic particles impart a magnetic fingerprint or signature for analytes of interest

The use of paramagnetic particle detection assays and system enhances the efficiency and accuracy of detection and relative to conventional immunodetection means. In biochemical separations applications, colloidal paramagnetic-particle labels utilizes the ability of antibodies to selectively link the analyte of interest to the magnetic nanoparticle.

### Detector

The invention may employ various functional means of detectors for the detection of analytes (e.g. hormones and hormone metabolites), including those described herein or known in the art. A detector for detecting analytes of interest may be utilized, for example, in a laboratory setting or in a home or field location. Certain embodiments are directed to a portable detector capable of use for measuring the excretion rate of particular metabolites. In other embodiments, the detector is not portable. The detector, whether portable or not, may be in communication with a database. The database may be electronic, for example computer or internet based. Thus, in certain embodiments a portable detector is utilized that is in communication with an electronic database comprising historical values of excretion rates for analytes of interest, including one or more estrogen metabolite and/or one or more progesterone metabolite.

Instruments useful for the detection, monitoring and/or analysis of biochemical analytes based on the detection of superparamagnetic particles are known in the art. Representative instruments include, for example, Magnetic Assay Reader (MAR) from Quantum Design, San Diego Calif.; as well as those described elsewhere, see WO 95/13531 to Catt et al., EP-A-833145 to Catt et al., U.S. Pat. No. 6,046,585 to Simmonds, U.S. Pat. No. 6,275,031 to Simmonds, U.S. Pat. No. 6,437,563 Simmonds et al., US Pat. App. No. 20040214347 to LaBorde et al., and U.S. Pat. No. 6,607,922 to LaBorde, the contents of each of which is incorporated in its entirety by reference. The superparamagnetic particles are typically bound to the analyte in a sandwich assay format. The detector can for example, measure the local magnetic field expressed by the total mass of magnetic particles in the immune complexes trapped in the detection region. Then, by way of an empirically established calibration curve, the resultant value may be correlated to the number of molecules of interest. Representative instruments are adaptable to existing assay formats and chemistry techniques including, for example, lateral flow membranes, DNA arrays, and dipstick assays.

Magnetic particles may be coupled to target particles by conventional methods to create magnetic bound complex samples. The target particles may include atoms, individual molecules and biological cells, among others. The magnetic bound complex samples are deposited in accumulations of several to several hundred particles at predetermined locations.

### Database and System

In another aspect, a fertility monitoring system is provided. The invention may employ various functional means of databases, hardware and software and systems for monitoring analytes and monitoring fertility, including those described herein or known in the art. Ovarian monitoring systems may take the form of an entirely hardware embodiment, an entirely software embodiment or an embodiment combining software and hardware aspects. They may be embodied, for example, as a computer program product on a computer-readable storage medium having computer-readable program code means embodied in the medium. Any suitable computer readable medium may be utilized including hard disks, CD-ROMs, optical storage devices, or magnetic storage devices.

Embodiments of the invention are described below with reference to flowchart illustrations of methods, apparatus (systems) and computer program products. Each block of the flowchart illustrations, and combinations of blocks in the flowchart illustrations, can be implemented by computer program instructions. These computer program instructions may be loaded onto a general purpose computer, special purpose computer, or other programmable data processing apparatus to produce a machine, which may be combined with the analyte detection system into a single device, such that the instructions which execute on the computer or other programmable data processing apparatus create means for implementing the functions specified in the flowchart block or blocks.

Computer program instructions may also be stored in a computer-usable memory that can direct a computer or other programmable data processing apparatus to function in a particular manner, such that the instructions stored in the computer-usable memory produce an article of manufacture including instruction means which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

Accordingly, blocks of the flowchart illustrations support combinations of means for performing the specified functions, combinations of steps for performing the specified functions and program instruction means for performing the specified functions. It will also be understood that each block of the flowchart illustrations, and combinations of blocks in the flowchart illustrations, can be implemented by special purpose hardware-based computer systems which perform the specified functions or steps, or combinations of special purpose hardware and computer instructions.

Computer programs suitable for implementing the present invention may be written in various object-oriented programming languages, such as Delphi and Java®. However, other object oriented programming languages, such as C++ and Smalltalk, as well as conventional programming languages, such as FORTRAN or COBOL, are contemplated as within the scope of the invention.

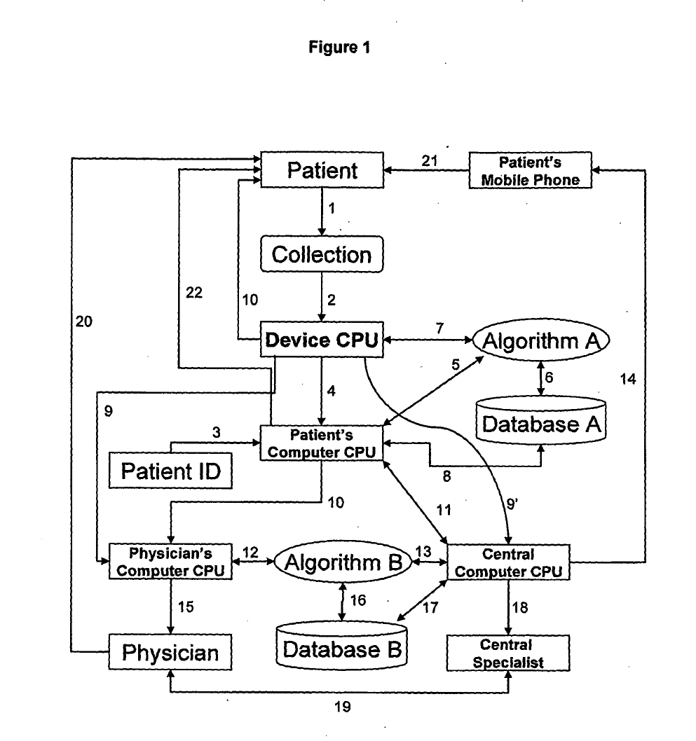

An embodiment of a system provided for obtaining, analyzing, storing, and transmitting fertility status assessment data of remotely located subjects in need of fertility management according to the present invention is schematically illustrated in FIG. 1. As shown, a plurality of remotely located subject monitor systems (SMS) are configured to establish communications directly with a central data processing system (CDPS) via communications links. The communication links can be a device selected from the group comprising a transmitter, a beeper, a receiver, a telephone, a modem, a cellular phone, a cable, an internet connection, a World Wide Web link, a television, a closed circuit monitor, a computer, a display screen, a telephone answering machine, facsimile machine, or a printer.

A plurality of remotely located healthcare provider central processing units (CPU's) are configured to establish healthcare provider-CPU communications with the CDPS server via communication links. In certain embodiments, the communication link is an internet or intranet link. In other embodiments, the communication link is a mobile telephony text messaging service. It is envisaged that other communication modes may be employed as they become available.

A SMS or CDPS server or other apparatus configured to execute program code embodied within computer usable media may operate as means for performing the various functions and methods of the various operations of the present invention. Embodiments of the invention may be used with various client-server communications protocols, including but not limited to specific protocols such as TCP/IP protocol.

Several embodiments are described herein with respect to ovarian cycle monitoring and fertility management. However, assays for a wide variety of medical conditions where monitoring and assessment of physiologic and/or biologic parameters are needed to facilitate or achieve clinical or therapeutic efficacy are also contemplated.

A SMS serves as primary means for collecting data from a subject and as means for case managers or health care provider to interface with a subject. Representative features of a SMS may include, for example, small size or portability, data processing capabilities and built-in or attachable external means for communication with linked components, data collection capability from bodily fluids, data collection capability directed to subject supplied data on health status, and monitoring capability of subject compliance to medical/fertility regime. A SMS may also function to allow two-way communication with CDPS server. A SMS may also function to analyze subject data collected and deliver live or pre-recorded responses and/or fertility management recommendations based on a physician or health care professionals instructions. A SMS may provide capability for downloading subject data to CDPS server at specified time intervals or in real time, capability for communicating messages, updates to physician or healthcare provider, instructions and fertility management regimens, fixed or contingent self-monitoring schedules, or other feedback from CDPS server.

Subject data collected via a SMS may include physiologic data (e.g., urinary metabolite, blood glucose measure, body temperature, and the like) or behavioral data (e.g., assessments related to diet, exercise, stress, the presence of illness). In certain embodiments, the subject data collected is urinary metabolite data. In certain embodiments, the SMS comprises an algorithm directed to a particular subject's reproductive condition in order to optimize efficacy of the specific fertility regimen. In certain embodiments, the SMS may be configured to make automatic adjustments to a subject's self-monitoring and fertility management regimen based on subject-entered data. In certain embodiments, the SMS may also contain a database to help subjects evaluate the effects of concurrent therapy for other non-fertility indications which might affect the fertility or ovulation cycle of the subject.

In certain embodiments, subjects are responsible for recording data within their SMS and transmitting the data to a CDPS server on a regular basis. In other embodiments, transmission of data to a CDPS server is highly automated and requires little or no input from a subject. In certain embodiments, a subject can use the system by plugging a SMS into a standard telephone jack and, with the press of a button, establish communications with a CDPS server CPU. Each SMS may have the ability to prompt subjects when data transmissions are required, and to initiate and complete data transmissions using alerting devices such as, for example, an alarm-driven timer.

In other embodiments, the SMS contains a user interface for displaying text, graphics, user-prompts, and various other information. In certain other embodiments, the SMS user interface may serve as the primary means of communication between the CDPS server and the subject. In certain embodiments, the SMS may also be configured to notify subjects of transmission schedules to the CDPS server; to notify subjects of urgent conditions related to fertility or otherwise to promptly seek medical attention; and to provide motivational feedback to subjects based upon past performance (e.g., reward subjects for keeping on schedule with data recordings and transmissions of data to a CDPS server). A suitable SMS for monitoring fertility management data of subjects is manufactured by Quantum Design (San Diego). Other representative features of the SMS may include, for example, systems and subsystems as those described in U.S. Pat. No. 6,046,585 to Simmonds; U.S. Pat. No. 6,275,031 to Simmonds; U.S. Pat. No. 6,437,563 to Simmonds et al., U.S. Pat. No. 6,607,922 to LaBorde; and US Pat. App. No. 20040214347 to LaBorde et al. Embodiments of the SMS may include, for example, a display, a keyboard, an analyte meter; internal data storage, internally stored fertility monitoring algorithm and/or software, and a data processor or CPU for operating the SMS and for communicating with a CDPS server. In certain embodiments, the SMS uses subject-entered data and internal software to continuously monitor ovulation status by measurement of metabolic byproducts such as, for example, urinary metabolites.

In some embodiments, when the SMS analyte meter is used to record fertility status-associated values, the internal software may query the subject for various information including, but not limited to, health status, diet, exercise, and medications taken. In some embodiments, the SMS internal software is menu-driven for ease-of-use by subjects. In certain embodiments, the data entered within the fertility monitoring SMS is stored with date and time information and can be alarm initiated (e.g. a subject or SMS can be prompted to perform a task or function). In certain embodiments, the SMS internal software analyzes the entered data and continuously informs the subject of her fertility status and prescribed regimen. In certain embodiments, the SMS internal software calculates adjustments for a subject's ovulation variation according to a physician or health care professional's prescription as applied to the data entered into the SMS by the subject.

In certain embodiments, the internal software of a SMS is configurable by a case manager via a CDPS server. A case manager can make adjustments to a subject's fertility management regimen and to a subject's fixed or contingent self-monitoring schedules. These adjustments can be made automatically within a SMS during routine data transfer to a CDPS server. In addition to providing fertility management, a SMS can be used to remind subjects to schedule appointments for important examinations.

In certain embodiments, the fertility management algorithm allows a physician or other health care professional to specify retrospective and/or supplemental adjustment regimens. In certain embodiments, the SMS contains a database of medication interaction information and is configured to allow a subject to query the database for information related to the subject's use of multiple medications. In certain embodiments, the SMS may be configured to communicate with an external database which may contain, for example, medication interaction information, specific historical fertility data profile for each subject, and historical fertility profiles for populations of subjects. In certain embodiments, the subject may query a database located within a CDPS server when communications are established between the SMS and the CDPS server. A SMS may also be configured to allow a subject to establish communications with other external databases.

Other features of a SMS may include connection slots for connecting a SMS to various peripheral devices; connection devices to land line telephone systems; and infrared ports for communications with peripheral devices. Additional SMS features for subjects in need of fertility managements are disclosed in U.S. Pat. Nos. 6,046,585 to Simmonds; 6,275,031 Simmonds; 6,437,563 to Simmonds et al; and 6,607,922 to LaBorde; as well as U.S. Pat. App. No. 20040214347 to LaBorde et al., which are incorporated herein by reference in their entirety.

Communications modalities for the SMS are not limited to land line telephone communications with a CDPS server. In certain embodiments, the SMS may communicate with a CDPS server using various communications technologies, without limitation. For example, a SMS may incorporate wireless communications technology for communicating with a CDPS server. In certain embodiments, the SMS may also incorporate direct satellite communications technology for communicating with a CDPS server.

Data entered into a SMS by a subject is transmitted to a central data processing system CDPS via communication means contemplated herein. It is understood that a CDPS server may be one or more data processing devices arranged in a network. Preferably, a direct communications connection is established between a SMS and a CDPS server. Alternatively, an indirect communications connection may be established between a SMS and the CDPS server via the Internet or other network described herein. A communications server is preferably utilized to handle inbound and outbound communications between a SMS and the CDPS server, as would be understood by those skilled in the art of client-server communications. The term CDPS server, as used herein, includes databases for storing and manipulating subject data as well as other server functions including, but not limited to, web servers, application servers, e-mail servers, fax servers, AVM servers, and the like.

In certain embodiments, a CDPS server analyzes and stores data transmitted from each subject SMS. This data is made available to authorized case managers or central specialist who can access the data via the internet, intranet, or other modes of communication described or contemplated herein. In particular, a CDPS server identifies and prioritizes subject fertility issues using the data transmitted from the subject SMS. This allows case managers to focus their attention first on subjects with urgent fertility problems or subjects in need of taking immediate action. In certain embodiments, a CDPS server performs real-time analysis on data as it is being transmitted from a SMS to identify fertility-related emergency situations that require immediate attention. If such an emergency is identified, a subject can be immediately notified via communications from a CDPS server to a SMS, without the intervention of a case manager. Alternatively, a case manager can be notified and the subject contacted directly via phone, e-mail, fax, or other modes of communication contemplated herein.

In certain embodiments, a CDPS server performs various other functions including allowing case managers to change the fertility planning program for subjects when a subject downloads data to a CDPS server. In certain embodiments, a CDPS server may include a “tickler system” for reminding case managers to verify that communications with subjects have occurred and for verifying that conditions requiring intervention or medical attention have been resolved. In certain embodiments, a CDPS server may also be configured to track subject supply usage automatically (e.g., test strips, pads or other detection devices) and this information may be used to provide just-in-time delivery of replacement supplies to a subject. In certain embodiments, a CDPS server may be configured to communicate with manufacturers and distributors of medical supplies utilized by subjects. By monitoring subject usage of supplies, orders can be placed with manufacturers and distributors directly via a CDPS server such that medical supplies can be delivered to subjects. In certain embodiments, a separate warehouse database may be added to a CDPS server to support complex analysis of subject data, and may also be used to review prescriptive changes made to a subject's fertility regimens and medication dosages.

Case Manager Clients (CPU)

In certain embodiments, case managers access a CDPS server via a case manager CPU (CMC) connected to the same network. The CMC preferably communicates with a CDPS server over an internet connection between the CMC and the CDPS server. In certain embodiments, data encryption may be utilized and other security methods may be implemented to transfer information between a SMS and CDPS server and between a CMC and the CDPS server or a SMS. Representative devices which may serve as CMCs for purposes of embodiments provided herein include, but are not limited to, desktop computers and portable computing devices, such as personal digital assistants (PDAs). In certain embodiments, a CMC preferably includes a central processing unit, a display, a pointing device, a keyboard, access to persistent data storage, and an Internet connection, for connecting to the internet. In certain embodiments, an internet connection may be made via a modem connected to traditional phone lines, an ISDN link, a T1 link, a T3 link, via cable television, via an ethernet network, and the like. In certain embodiments, an internet connection may be made via a third party, such as an “Internet Service Provider” (“ISP”). In certain embodiments, an internet connection may be made either by a direct connection of a CMC to the Internet or indirectly via another device connected to the internet. In the latter case, a CMC is typically connected to this device via a local or wide area network (LAN or WAN). In certain preferred embodiments, data transfer rates between a CMC and a CDPS server are equal to, or greater than, fourteen thousand four hundred baud (14,400 baud). However, lower data transfer rates may be utilized.

It is to be understood in the art that that various processors may be utilized to carry out the embodiments of the invention without being limited to those enumerated herein. Although a color display is preferable, a black and white display or standard broadcast or cable television monitor may be used. In certain embodiments, a CMC preferably utilizes either a Windows® 3.1, Windows 95®, Windows NT®, Unix®, Mac/Apple Operating Systems, or OS/2® operating system. However, it is to be understood that a terminal not having computational capability, such as an IBM® 3270 terminal or a network computer (NC), or having limited computational capability, such as a network PC (Net PC) may be utilized in accordance with embodiments provided herein for accessing the internet in a client capacity.

In certain embodiments, a case manager accesses a CDPS server via a CMC to review the fertility conditions of multiple subjects. In certain embodiments, case managers preferably are able to review, via information downloaded from a CDPS server, all subject activity and data for their assigned subjects including data transmission history, prescription review, analysis and adjustment. A CMC allows a case manager to review subject data in various formats, including a hierarchical, problem-oriented format wherein subjects with medical conditions requiring immediate attention are presented foremost. In certain embodiments, a CMC may also allow a case manager to add, edit, and delete certain subject data stored in a CDPS server. In certain embodiments, a CMC also can interface directly with each SMS to provide a subject with information and to modify condition-specific software contained therein.

### System Security

Access to a system for monitoring fertility status assessment data of remotely located subjects in need of fertility management according to certain embodiments may be controlled using logon security which provides case managers and other users with certain circumscribed privileges to examine and/or edit data. These rights can limit certain users ability to examine confidential clinical health data, and may also be employed to limit the ability to edit any clinical data or make changes to specific fields in a subject's fertility-related regimen or adjustment algorithm. Similar access control may be applied to the data, at various levels, which define subjects' fertility or medical conditions. In certain embodiments, flexible configuration and associated security may be an element of a system for monitoring fertility conditions of remotely located subjects that permeates many of the subsystems.

Default values and classifications for many values may be provided at the system level. Default values may be modified in a hierarchical manner, and may be controlled in part by access rights of a user, to a permit uniqueness at various levels. In certain embodiments, detection devices many be encoded with unique identifying numbers, such as for example, test strip ID number or bar codes.

### Operations

In certain embodiments, subject data are obtained by a CDPS server from a SMS. A CDPS server analyzes the obtained data to identify subjects with fertility conditions requiring urgent attention. A CDPS server may prioritize the identified subject conditions according to urgency or severity. A CDPS server may display to a case manager (or other user), via a client in communication with the CDPS server, a selectable list of subjects with identified fertility conditions arranged in priority order. In certain embodiments, a CDPS server may provide to a case manager, via a client, options for treating each identified fertility condition. In certain embodiments, physician-prescribed or health care professional-prescribed fertility regimen or modification thereof may be implemented based on subject data obtained from a SMS. In certain embodiments, fertility management information may be communicated directly to a subject or to a subject's SMS by a case manager via a client in communication with a central data processing system.

### Obtaining Data From SMS

In preferred embodiments, when a CDPS server obtains subject data from a SMS, fertility data analysis may be performed by algorithm B. In certain embodiments, data transmitted to a CDPS server is analyzed substantially simultaneously with transmission of the data for the purposes of identifying “emergency” fertility conditions requiring immediate attention. Preferably, this analysis is performed while communications are still established between a CDPS server and a SMS transmitting the data. If emergency conditions are not identified, data obtained from a SMS is stored within a CDPS server database (database B) for later analysis and retrieval. In certain embodiments, a database may contain information obtained for comparative analysis over a population of subjects. In certain embodiments, if urgent conditions are identified, instructions are downloaded to the SMS regarding what actions should be taken by the subject. For example, the subject may be instructed to immediately take a specific action or to immediately seek medical attention. In certain embodiments, the CDPS server may communicate a new fertility regimen SMS, or to the subject via telephone, AVM, e-mail, facsimile transmission, and the like. In addition, changes may also be made to fertility algorithms stored within a SMS or within the CDPS server, such that a subject's next course of action is changed in response to the identified urgent condition. Furthermore, changes may also be made to a subject's fixed or contingent self-monitoring schedules. The data obtained from a SMS is then stored within a CDPS server database for later analysis and retrieval.

### Analyzing Subject Data

In certain embodiments, a case manager is provided with various options for resolving one or more fertility conditions. In certain embodiments, a case manager may be presented with an option to contact a subject. The case manager may contact a subject, via telephone, e-mail, AVM and facsimile transmission. A case manager may be presented with an option to adjust a fertility regimen or self-monitoring schedule, either within a subject's SMS or the CDPS server. If a case manager decides to adjust a regimen within a subject's SMS, the present invention facilitates this modification though a CDPS server the next time communications are established between the CDPS server and the subject's SMS. In certain embodiments, a subject may be prompted to establish communications between her SMS and a CDPS server to receive modifications made by a case manager.

In certain embodiments, a case manager may be presented with an option to schedule a subject for a visit with a health care provider or with an option to seek expert fertility medical input. If these options are selected, the present invention facilitates scheduling a subject to visit a health care provider or obtaining input from a medical expert. In certain embodiments, a case manager may provide that no action is required for a particular fertility condition and may remove an identified fertility condition from an active condition list for a particular subject after reviewing available data. In certain, embodiments, the operations are performed by a CDPS server immediately after transmission of data from a SMS to the CDPS server.

### Communicating Treatment-Information to Subject

In certain embodiments, a case manager may also select and/or compose messages to be downloaded to a subject's SMS, or transmitted via telephone, AVM, e-mail and facsimile transmission, which are designed to reinforce correct behaviors or alter maladaptive behaviors.

In certain embodiments, a case manager may also compose a message asking a subject to schedule an office visit with a physician or health care professional, and may also alter a SMS transmission schedule (which may take affect following the next transmission). In certain embodiments, special messages related to scheduling office appointments ask the subject to make an appointment with a named professional and provide his or her phone number. In certain embodiments, the SMS may query the subject on a daily basis concerning whether the appointment has been made, and then solicit the appointment date for uploading to the CDPS. After the appointment date has passed, the SMS can query the subject to ascertain if the appointment was actually kept.

In cases where case managers have questions concerning a subject's fertility condition or prescription, case managers may seek input from medical experts using a user interface. In certain embodiments, a case manager may communicate with subjects in various ways, such as via telephone, e-mail, AVM and facsimile transmission. In certain embodiments, the present invention provides pre-composed text for inclusion in text-based communications such as text-messaging, letters, faxes and e-mail directed to a subject. In certain embodiments, case managers may utilize the present invention to facilitate and track subject appointments with clinic personnel or other providers involved in health care. Once a decision is made to schedule a subject appointment, a system task reminder may be generated that requires periodic follow-up until a record of a scheduled appointment time is input into a CDPS server. A case manager may employ a subject's SMS to prompt the subject to make an appointment, and subsequently query the subject for the appointment date once it has been made. Other contact methods may also be employed to prompt a subject to make an appointment and subsequently to inform the case manager concerning the date (e.g., via e-mail, AVM, telephone, and facsimile transmission). In certain embodiments, a SMS may also be used to verify appointment compliance. In certain embodiments, the present invention also tracks appointment compliance (e.g., whether a subject kept his/her appointments). In certain embodiments, healthcare providers can be sent communications to confirm whenever an appointment has been kept by a subject and to supply associated lab or examination data to a CDPS server. To track appointment compliance with providers who cannot directly access a CDPS server, a case manager may generate correspondence and associated follow-up reminders in order to obtain confirmation and associated clinical data if desired.

In certain embodiments, statistical analysis may optionally be performed that utilizes pattern analysis, multiple regression, time series and other types of analyses to compare current subject data sets to earlier data and to data of other appropriate subjects.

In certain embodiments, a computer program is used to enter data daily, and to obtain a graphical display. The computer program has algorithms to interpret data for fertility monitoring, as well menus such that the user has access to help on the details of running the test. The program has a user mode and an advisor mode. The user mode allows the user to email her file to an advisor who can open it up in the advisor copy and see the cycle unfolding. The advisor mode also has access to a database. Another aspect of the invention includes a system for communication of data between users and a central advisory facility re fertility status of an individual. Also provided are a web based interface that has a data interpretation algorithm and client billing interfaces.

### Methods

The inventions provided herein may be used to determine the ovulation cycle status or measure fertility in a female animal (e.g. mammals, birds, reptiles, amphibians, fish, etc.). Typically the animal is a mammal, including for example humans, domestic animals, non domestic animals, farm animals (e.g. bovine or equine) and pets.

For the assays provided herein, it is not necessary for the female animal to assume regular menstrual cycles. In certain embodiments, information from previous menstrual cycles is not used in monitoring fertility. The assays provided herein can be used, for example, for normal cycle pregnancy achievement or avoidance, return to fertility after breastfeeding, approaching the menopause, for management of infertility, gonadotrophin therapy, etc.

Certain embodiments are directed to a rapid non-invasive laboratory accurate tests which are useful for monitoring the ovarian cycle. Estrone glucuronide and pregnanediol glucuronide tests provided herein are indicators as to follicle growth and corpus luteum establishment. For example, if the estrone glucuronide excretion rate increases, then there is a high degree of certainty that a follicle is growing. If the pregnanediol glucuronide excretion rate increases, there is a high degree of certainty that luteinisation has occurred to at least some extent. A range of threshold values for pregnanediol glucuronide which act as indicators of the menstrual cycle are used in certain embodiments. Threshold values described in Vigil, P., et al., Fertility and Sterility, S167, (1998) and Blackwell, L. F., et al., Steroids, 63, 5. (1998), incorporated by reference herein, may be used.

In certain embodiments, estrone glucuronide and pregnanediol glucuronide are each detected or quantified. In other embodiments, the only analyte that is measured is pregnanediol glucuronide. For most applications described herein, estrone glucuronide and pregnanediol glucuronide are the most useful analytes to measure. However, other analytes may be monitored. Follicle stimulating hormone (FSH) and luteinizing hormone (LH) may rise but unless the ovarian events are confirmed by, for example, ultrasound, or a functional test, the following events are assumed but not proven. It is possible for example to have an luteinizing hormone surge/rise with no ovulation. It is also possible to have no detectable urinary luteinizing hormone surge/rise but ovulation still occurs as shown by the pregnanediol glucuronide and estrone glucuronide excretion patterns.

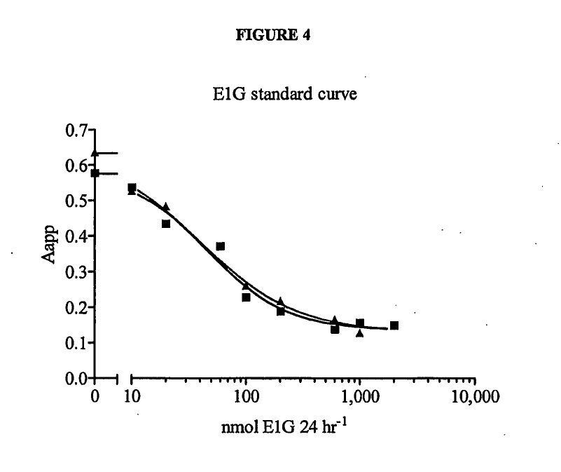

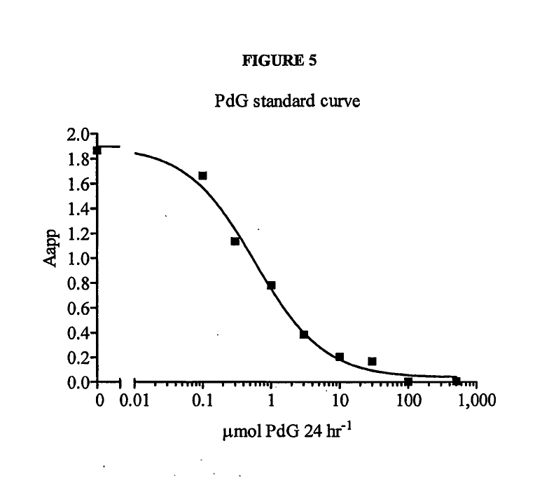

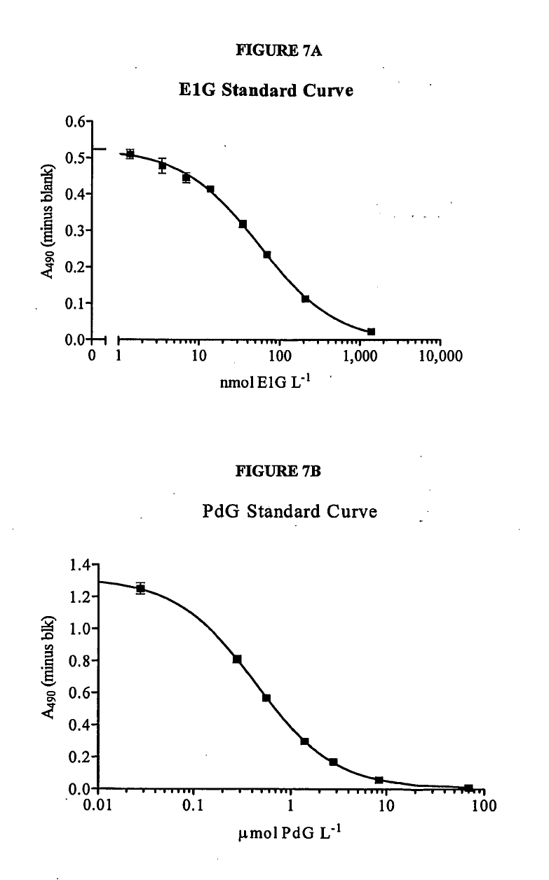

In another aspect, the excretion rates of certain hormone metabolites from urine are determined. Excretion rates or other data obtained from one or more time point can be compared to a compilation of data obtained, for example, from a particular animal., a particular individual., or a set of individuals. Such data compilations may be in the form of, for example, a reference curve or graph, an electronic database, or the like. Data compilations of excretion rates for particular analytes, in particular animal species, and under a variety of conditions are provided herein. For example, reference curves for estrone glucuronide and pregnanediol glucuronide obtained from cyclic urine samples in humans and cows are provided herein (see Examples 4 and 6; FIG. 4, FIG. 5, FIG. 7A and FIG. 7B). Reference curves in humans described Brown, J. B., et al., Progr. Biol. Clin. Res., 285, 119. (1988), incorporated by reference herein, may be utilized.

Species specific analyte databases are unique and particularly suitable for providing accurate data for determining the ovulation cycle status in particular animals. For example, in certain embodiments the excretion rates for estrone glucuronide and pregnanediol glucuronide for at least one bovine ovulation cycle are provided. This compilation of bovine estrogen metabolite and progesterone metabolite values is also provided as an electronic database.

In certain embodiments, the body fluid used for the samples is urine and it is collected over a specified interval of time. Suitable time intervals include, for example, 3 hours, 4 hours, 5 hours, 6 hours, 7 hours, 8 hours, 9 hours, 10 hours, and up to 24 hours. Other intervals of time may be used, including fractions of the time intervals listed, or greater time intervals. In some embodiments, the urine is collected over at least a 3 hour time period. In other embodiments, urine is collected over at least a 3 hour time period and the volume of the urine sample is measured, and then adjusted to a normalized volume that corresponds to the time interval of the collection period. The step of normalizing the sample volume may be performed prior to quantifying the excretion rate of particular analytes. For example, in certain embodiments, the volume of a urine sample is normalized prior to determining the excretion rates for an estrogen metabolite and a progesterone metabolite.

In particular embodiments, urine is collected from a human female over at least a 3 hour time period and the volume is adjusted to a normalized volume equal to about 150 ml/hr. Other sample volume adjustments are possible, including for example adjusting collecting urine from a human female and adjusting the normalized volume equal to about 100 ml/hr, 200 ml/hr, 250 ml/hr, 300 ml/hr, 350 ml/hr, 400 ml hour, 500 ml/hr, 1000 ml/hr, etc. Additional volume adjustments or dilutions may be made. The sample volume and/or excretion rate may be adjusted or normalized by a computer algorithm. Where body fluid (e.g. urine, milk) is collected from a non human female, any sample volume adjustments is typically dependent on the animal and the body fluid.

In certain embodiments, excretion rates of an estrogen metabolite and a progesterone metabolite are quantified daily for a set interval of time. Suitable intervals of time include, for example, from between about 2 to about 4 days, from about 2 to about 5 days, from about 2 to about 6 days, from about 2 to about 7 days, from about 2 to about 8 days, from about 2 to about 9 days, from about 2 to about 10 days, from about 2 to about 12 days, from about 2 to about 15 days, from about 2 to about 20 days, from about 2 to about 25 days, from about 2 to about 28, and from about 2 to about 30 days.

In another aspect, information obtained regarding the ovulation cycle status is used to measure fertility in a female animal., including determining a time frame for optimal fertility within a menstrual cycle of said female subject. Thus, various aspects of the invention are useful for determining a time frame for optimal fertility for performing an in vitro fertilization of said female subject.

In certain embodiments, excretion rates of an estrogen metabolite and a progesterone metabolite are quantified daily for a set interval of time. Suitable intervals of time include, for example, from between about 2 to about 4 days, from about 2 to about 5 days, from about 2 to about 6 days, from about 2 to about 7 days, from about 2 to about 8 days, from about 2 to about 9 days, from about 2 to about 10 days, from about 2 to about 12 days, from about 2 to about 15 days, from about 2 to about 20 days, from about 2 to about 25 days, from about 2 to about 28, and from about 2 to about 30 days.

In another aspect, information obtained regarding the ovulation cycle status is used to measure fertility in a female animal., including determining a time frame for optimal fertility within a menstrual cycle of said female subject. Thus, various aspects of the invention are useful for determining a time frame for optimal fertility for performing an in vitro fertilization of said female subject.

In certain embodiments, one or more hormone metabolite is measured for one or more days, or on a daily basis for a desired period of time. Algorithms are provided herein that are used to analyze, estrone glucuronide and pregnanediol glucuronide excretion rates, for example from one time point to one or more other time points, to provide a reliable determination as to whether a statistically significant increase in estrone glucuronide has occurred. Algorithms can be used to set one or more threshold values for analyzing estrone glucuronide and pregnanediol glucuronide levels determined from strip assays. From these thresholds, various predications regarding the fertility status can be made in a point-of-care, home, or clinical setting with an accuracy that is equivalent to that of test performed by a clinical laboratory.

A PdG excretion rate threshold can be determined such that it applies to substantially all women because it marks a level of PdG excretion which is virtually never reached or exceeded and which is subsequently followed by an ovulation without an intervening menstrual bleed. In certain embodiments, this is set at an excretion rate of 7 μmol/24 h for PdG. This value is used as a threshold for marking the beginning of luteal phase infertility. When the PdG excretion rate equals or exceeds this value a determination is made that the cycle is no longer fertile and no further testing is required. (see Blackwell, L. F., et al. Steroids, 63, 5. (1998), incorporated by reference herein)

Threshold values for the excretion rate of E1G are also useful in certain embodiments. A statistically significant rise in the excretion rate of E1G followed by a fall in E1G excretion rate may indicate the presence of a growing follicle as described by Blackwell, L. F. et al., Steroids, 57, 554 (1992), incorporated by reference herein. Once a follicle commences to grow it has two possible fates; to continue to ovulation at which time there is a sharp drop in the E1G excretion rate, or by atresia in which case there may also be a sharp drop in E1G excretion rate. If the PdG excretion rate increases to equal or exceed a predetermined threshold value, a determination is made that ovulation has likely occurred. This can be confirmed by continued monitoring of PdG until the level exceeds a predetermined level, which may be set at 10 μmol/24 h for PdG.

In practice, the use of thresholds for E1G excretion rates are more difficult to apply in an assay because E1G excretion rates are more variable amongst different women. Also, the fraction of ovarian oestradiol that is converted into estrone glucuronide varies between individuals. In certain embodiments, these problems are obviated by determining a E1G excretion rate for an individual woman to use this as the marker for the beginning of the fertile period as described in Brown, J. B. and Blackwell, L. F., The Ovarian Monitor. Instruction Manual. Ovulation Method Reference and Research Centre. Melbourne, Australia (ISBN 0 908482 03 05). (1989), incorporated by reference herein.

In certain embodiments, excretion rates for both E1G and PdG are measured in tandem to provide a determination of the general position in the fertility spectrum. If both E1G and PdG excretion rates are both below a predetermined threshold, a determination can be made that the woman is in the early follicular infertile period or is in a state of amenhorrea (the absence of a menstrual period). Once a predetermined E1G threshold is exceeded, a determination can be made that the woman is in the fertile phase of the cycle if a predetermined PdG threshold is not exceeded. Once this predetermined PdG threshold is exceeded, a determination can be made that the woman is in the luteal phase infertile period and can no longer conceive in that cycle.

In certain embodiments, excretion rates for particular analytes are stored in a central database that is in communication with a analyte monitoring device. The analyte monitoring device may, for example, communicate with a central database through a wired or wireless connection.

In certain embodiments, if the excretion rates for estrone glucuronide and pregnanediol glucuronide are both at or below a predetermined threshold, a determination is made that the female is in an infertile state. If a statistically significant rise in estrone glucuronide excretion occurs or the excretion rate is elevated to or above a certain threshold but pregnanediol glucuronide is at or below a predetermined threshold, a determination is made that the female is in a potentially fertile state. If pregnanediol glucuronide has risen to or above a threshold level, than a determination is made that the female has ovulated. Pregnanediol glucuronide excretion rates will also be indicative of the equality of the corpus luteum, deficient, adequate, or short luteal phase.

One method of determining analyte excretion rates provided herein comprises applying a urine volume adjustment. Certain embodiments utilize a correction for urine volume. Urine volume corrections can be made by diluting a urine sample in situ. Such methods may initially include instructing a users how to collect a timed urine sample. While analyte excretion rates over a 24 hour period may be useful for comparisons, a collection period of less than 24 hours may be used. A female subject (e.g. client/user) may collect all the urine except the first voiding at the start of the time period, but including the last voiding at the end of the time period, into a container that is calibrated according to hours of collection. The sample is then diluted with water (tap or distilled water can be provided) to 150 ml/hr of collection to the nearest quarter hour. Thus in certain embodiments, a 3.5 hour collection will be diluted to about 525 ml and only a small aliquot of the diluted sample need be retained for the assay.

In one embodiment, the fertility monitor comprises a sample dispenser that provides a fixed sample volume to a test strip. In another embodiment, the fertility monitor comprises a sample dispenser that provides an adjusted or normalized sample volume to a test strip. In alternative embodiments, urine volume corrections are made by applying an algorithm to adjust values to obtain excretion rates. The algorithm may be, for example, a computer program or internet based. For example, a computer program can be used by a home user for providing information on the use of the system and to permit display of the data on a daily basis.

To correct for urine volume fluctuations it is necessary to make a measurement from which the urine is normalised. In one embodiment this is achieved by collecting all of the urine over a fixed time period and diluting to a constant volume so that all urines have the same total volume per hour of collection. For animals this is not possible. Hence in these cases a measurement must be made by which the hormone concentration data may be normalised.

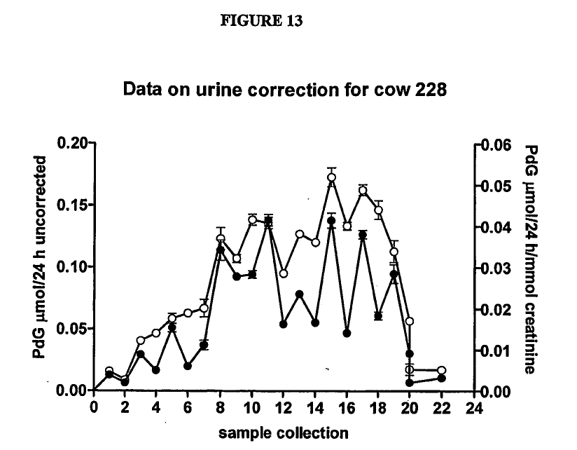

One such measurement is creatinine. This can be measured by the Jaffe reaction and the hormone concentration for each day divided by the amount of creatinine in the urine. FIG. 13 shows the smoothing effect on the PdG profile by such calculations.

In another aspect, a urine sample volume correction is made with reference to the specific gravity determination made for a sample. This measurement can be taken by an optional component of the monitoring device provided herein. The specific gravity of the sample is determined through a measurement of the sample's refractive index. The refractive index is used to calculate the concentration of the sample, which is used to calculate the analyte excretion rate.

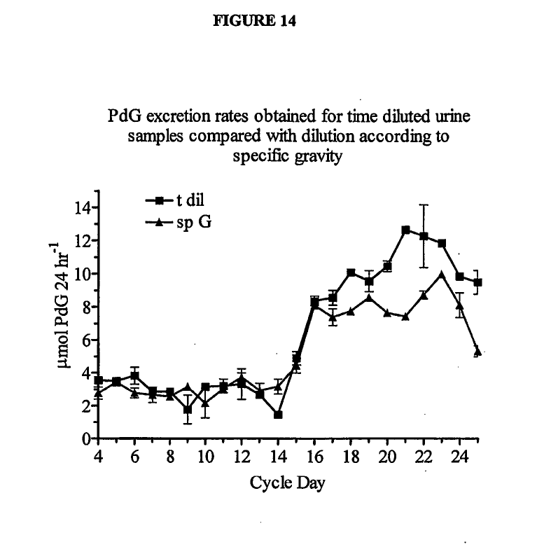

An alternative is to use specific gravity to make the correction. For example if the average specific gravity of a urine sample diluted to 150 ml/h is known then a dilution factor can be worked out for any urine sample on the basis of its specific gravity. This was done for a menstrual cycle and the PdG excretion rates determined on this basis compared with the usual time diluted samples. The results are shown in the FIG. 14.

### Dairy Procedures

In another aspect, ovulation cycle monitoring methods and devices described herein are used on non-human animal species. In certain embodiments, a method is provided for determining the fertility status of an animal comprising detecting, monitoring or analyzing the respective estrus and ovulation cycles. In the dairy industry, after heifers reach puberty (first ovulation) or following the postpartum anestrous period (a period of no estrous cycles) in cows, a period of estrous cycling begins. Estrous cycles give a heifer or cow a chance to become pregnant about every 21 days. During each estrous cycle, follicles develop in wave-like patterns, which are controlled by changes in hormone concentrations. In addition, the corpus luteum (CL) develops following ovulation of a follicle. While it is present, this corpus luteum CL inhibits other follicles from ovulating. The length of each estrous cycle is measured by the number of days between each standing estrus.

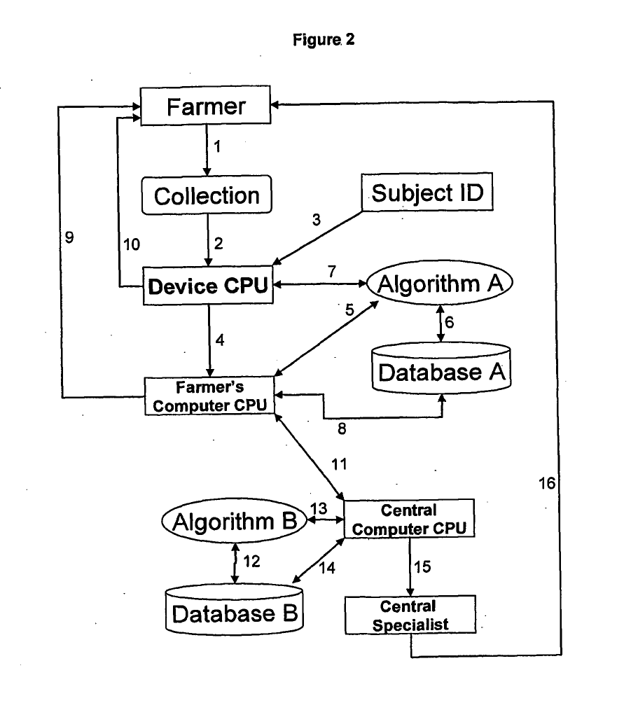

Anestrus occurs when an animal does not exhibit normal estrous cycles. This occurs in heifers before they reach puberty and in cows following parturition (calving). During an anestrous period, normal follicular waves occur, but standing estrus and ovulation do not occur. Therefore, during the anestrous period heifers or cows cannot become pregnant. Standing estrus, also referred to as standing heat, is the most visual sign of each estrous cycle. It is the period of time when a female is sexually receptive. Estrus in cattle usually lasts about 15 hours but can range from less than 6 hours to close to 24 hours. In cattle, the period of time when a female will stand and allow mounting by other animals is the sexually receptive period. A females enters standing estrus gradually. Prior to standing estrus she may appear nervous and restless (for example, walking a fence line in search of a bull or bawling more than usual). Prior to standing to be mounted by a bull or other cows, she will usually try to mount other animals. These signs will progress until standing estrus occurs. Other signs that a cow might be in standing estrus are a roughed up tailhead, a clear mucous discharge from the vagina, and a swollen vulva. However, the only conclusive sign that a cow is in estrus is standing to be mounted by other animals. Following standing estrus, the ovulatory follicle that is present typically ovulates, releasing the egg it contains. Rupture of the dominant follicle is referred to as ovulation and occurs between 24 and 32 hours after the onset of standing estrus. Following the release of an egg from an ovulatory follicle the egg will enter the female reproductive tract and be fertilized if the female has been mated. Following each standing estrus, a new estrous cycle will be initiated. In a normally cycling animal the interval between each standing estrus should be about 21 days (FIG. 2), but the range in normal estrous cycle length is from 17 to 24 days. When evaluating reproductive efficiency, it is important to realize that the interval between standing estrus can vary from 17 to 24 days. There is an abrupt drop in serum progesterone levels 3-4 days prior to the next (expected) oestrus. This is clearly seen in the urine by monitoring PdG excretion rates.

Certain compounds (e.g. hormones, metabolites, etc.) are abbreviated herein as follows: E1G—estrone glucuronide; PdG—pregnanediol glucuronide, PMP—paramagnetic particles, MES—2-(N-morpholino) ethanesulfonic acid, sodium salt, EDC—1-ethyl-3-(3-dimethylaminopropyl)carbodiimide, Mab—monoclonal antibody, Ab—antibody, BSA—bovine serum albumin, EDTA—ethylenediaminetetraacetic acid, PEG—polyethylene glycol, DCC—dicyclocarbodiimide, NHS—N-hyrdroxysuccinimide, DMF—dimethylformamide, T—test, C—control, MAR—Magnetic Assay Reader or Magnetic Assay Reading, SD—standard deviation, MA—radioimmunoassay, ELISA—enzyme linked immunosorbent assay, OM—Ovarian Monitor, LH—luteinising hormone, HMG—human menopausal gonadotrophin, IU—international units, GT—gonadotrophin, HCG—human chorionic gonadotrophin, EDO—estimated day of ovulation, ΔT—change in Ovarian Monitor transmission units, BIP—basic infertile pattern.

The following Examples are offered by way of illustration and not by way of limitation.

### Example 1

### Preparation of polyclonal anti-PdG 213-5 Antibody—Gold Conjugate

Polyclonal anti-PdG Ab 213-5 was partially purified by octanoic acid precipitation followed by an ammonium sulphate cut. The antibody was diluted 1/10 with 10 mM phosphate buffer, pH 7.4. The gold sample was from British Biocell International 40 nm microspere and adjusted to pH 7.8 with 0.02 M K₂CO₃. The gold solution (10 mL) was added to 100 μL of a 1/10 dilution of the Ab+400 μL 10 mM phosphate buffer (pH 7.4) and mixed by vortexing and left for 5-10 minutes at room temperature. Blocking buffer (300 μL of 10% BSA in 10 mM phosphate, pH 7.4 buffer) was added and the solution mixed by vigorous vortexing, and left for 10 minutes. The mixture was then centrifuged at 6,000 rpm for 1 hr, the supernatant was discarded and the precipitate washed 3 times with 1 mL of storage buffer (2% BSA in PBS with azide). The conjugate was resuspended in 1 mL storage buffer. Conjugation of antibodies with microspheres was carried out according to Henderson K. and Stewart J., Reprod Fertil. Dev, 12, 183-189 (2000), incorporated by reference herein.

### Example 2

### Methods for Pregnancy Avoidance and Pregnancy Achievement and in Humans

### Pregnancy Avoidance

A growing follicle signals its presence by an increasing daily excretion rate of E1G. Blackwell, L. F. and Brown J. B. Steroids, 57, 554 (1992), incorporated by reference. Ovulation is indicated by a peak in the E1G excretion rate and a rising PdG excretion rate. Blackwell, L. F., et al., Steroids, 63, 5. (1998), incorporated by reference. A corpus luteum is indicated by rapidly rising PdG excretion rates. The normal cycle consists of three sequential phases: (1) an infertile phase (I) of variable length when the ovaries are quiescent (or inactive), which is shown by continuing low rates of both E1G and PdG excretion; (2) a fertile phase (F) of variable length when an egg is growing in its life support system (the follicle), which is indicated by the first statistically significant rise in the E1G excretion rate while the PdG excretion rates remain low; and (3) a second infertile phase (IL) of fixed length (10-14 days) after ovulation when the follicle becomes a corpus luteum, which is indicated by a rapidly rising PdG excretion rate to exceed a threshold value of 7 mmol/24 h.

If the PdG excretion rate is still elevated 16-18 days later pregnancy is likely. The sequence will be I, F and then IL but only if ovulation occurs and the PdG excretion rates rise. The data is read from the strips by the paramagnetic reader and stored in the reader. The reader compares the band intensity of the strip with a standard or calibration curve which relates band intensities to the corresponding E1G or PdG concentration. The standard curve is stored in the reader and applies to the batch identification of the strips being used. A range of standard curves is typically applied. If the urine samples are timed and diluted to 150 ml/hr, the excretion rates are obtained directly from the standard curve which is established using timed and diluted urine samples. The readout from the strips is stored in the CPU of the reader and is stored as an array with the cycle day or the date as shown below for some PdG half strips (Table 1). The half strips have an absorbent pad, and incorporate both control and capture lines. The urine and antibody reagent are mixed in a well and the strips dipped into the wells. In contract, when full strips are used the antibody reagent is typically dried onto the conjugate pad and the strips are dipped into the urine as the sole liquid component.

TABLE 1
The PdG strip data stored in the CPU.

| Day | PdG strips μmol./24 h |
|---|---|
| 1.0 | 2.305506 |
| 2.0 | 3.739149 |
| 3.0 | 1.932238 |
| 4.0 | 1.18333 |
| 5.0 | 2.170635 |
| 6.0 | 2.119834 |
| 7.0 | 1.497935 |
| 8.0 | 0.4916423 |
| 9.0 | 0.8571002 |
| 10.0 | 1.172603 |
| 11.0 | 3.01097 |
| 12.0 | 1.517774 |
| 13.0 | 0.4000185 |
| 14.0 | 2.370969 |
| 15.0 | 3.092003 |
| 16.0 | 2.491832 |
| 17.0 | 2.079135 |
| 18.0 | 3.887302 |
| 19.0 | 8.253916 |
| 20.0 | 10.17638 |
| 21.0 | 6.525814 |
| 22.0 | 10.48178 |
| 23.0 | 13.19508 |
| 24.0 | 9.371401 |
| 25.0 | 12.90348 |
| 26.0 | 3.730723 |
| 27.0 | 8.376038 |
| 28.0 | 8.229706 |
| 29.0 | 5.375907 |

A method for monitoring a normal human menstrual cycle is performed by the following steps. (1) Enter the first day of bleeding (or the current bleeding day); (2) The monitor will signal on day 6 that testing should commence (very few pregnancies occur in the first 5-6 days of a cycle); (3) The test will signal F (fertile) or I (infertile); (4) If I is indicated, continue testing until F is indicated (by a statistically significant rise in the E1G excretion rate) and then discontinue for 5 days. If F already wait 5 days and go to step 5; (5) Recommence testing and continue until IL is signaled (by the rising PdG excretion rates); (6) Then cease testing until bleeding occurs when the sequence begins again.

Many variants of the “normal” cycle are possible and all are in fact usual. All are covered by the same testing system and the same principles. Anovulatory cycles are indicative of no rise in either the E1G or PdG excretion rates as measured by the strips. Test until I changes to F and then continue as above. Another variant of the normal cycle occurs when several follicles potentially start to grow and die before one ovulates (indicated by fluctuating E1G excretion rates above the baseline). In this case F will be indicated because the E1G excretion rate has risen but IL will not be indicated following this until the PdG excretion rate has risen. Hence testing may need to be continued for more days. One could wait 5 days after F has been signaled and if IL does not follow within the next two days wait another 5 days and test again and so on

### Pregnancy Achievement

As in the normal cycle but continue testing until the “mid-cycle” E1G peak is observed and time intercourse for the day that the E1G excretion rate drops from this value. The excretion rates measured from the strips will signal this as the most fertile day (Pk). Testing 18 days later may identify pregnancy. If the PdG excretion rates measured from the strips are still elevated, pregnancy will be indicated and a pregnancy test should be run. If pregnancy does not occur full cycle monitoring is recommended and the type of reduced fertility will be identified from the absolute levels of the PdG excretion rates measured by the test.

### Example 3

### Algorithm for the Identification of the First E1G Rise

A method of identification of the first rise in E1G for a normal cycle has been developed that is an adaptation of the Trigg's tracking signal algorithm described in Blackwell L. F. and Brown J. B., Steroids November; 57(11):554-62 (1992). The tracking signal algorithm has been modified to give a prospective detection of E1G rises. The method is performed by first determining four starting parameters. The starting parameters for this algorithm include: i) the initial value of the exponentially smoothed average (ESA(0)), ii) the initial value of the mean average deviation (MAD(0)), iii) the initial value of the forecast error (FE(0)) and iv) the initial value of the smoothed forecast error (SFE(0)). FE(0) and SFE(0) can be set to zero. Typically, ESA(0) and MAD(0) are calculated from the first 6 baseline days of the cycle if there is a baseline period. In this case the tracking signal can only give a warning after 6 days. If the value of ESA(0) is set as the first E1G excretion rate in the sequence of measurements and MAD(0) is set at an average value, either for the particular woman from previous cycles or from a population average, then a daily prospective analysis of the E1G data is given. The smoothing constant (α) is set at a value corresponding to a hypothetical baseline of 6 days (a value of 0.286 (N=6)). The statistical significance is set for the E1G data on the basis that it is better to recognize the first statistically significant rise in E1G excretion rate a day early rather than a day late. For the smoothing constant chosen a tracking signal of 0.72 represents 95% cumulative probability that a significant rise in E1G excretion has occurred (see Batty M, Operation research Quarterly, 20: 319-325 (1969), incorporated by reference herein).

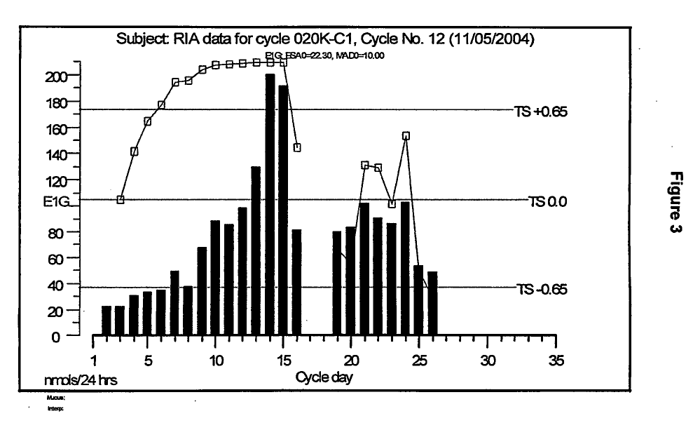

The identification of the first rise in E1G is illustrated in FIG. 3. The ESA(0) value was set at 22.3 μmol/24 h (the first value recorded for the E1G excretion rate and MAD(0)) was set at 10.0 which is an average value for the pre-ovulatory cycle data for this woman. A tracking signal of 0.85 was calculated for the E1G data on cycle day 7 which thus serves as the beginning of the potential fertile period (the day of the first statistically significant E1G rise at 95% confidence level). The tracking signal was calculated for each day of the cycle from the first day so the algorithm is truly prospective. No baseline calculation is needed. The rise in PdG excretion rate is measured simply by comparison with a threshold value of 7 μmol/24 h which applies to all women (Blackwell, L. F., et al., Steroids, 63, 5 (1998), incorporated by reference herein).

### Example 4

### Standard Curves

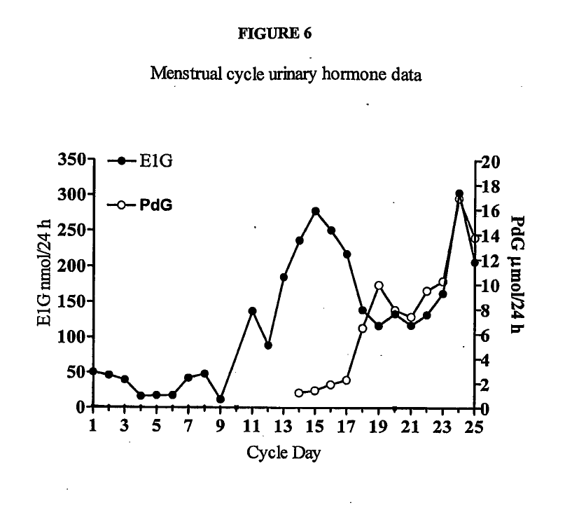

Standard curves for E1G and PdG for use with timed and diluted human urine samples are illustrated in FIGS. 4 and 5. FIG. 4 is a standard curve for E1G obtained from strips which have been sprayed with an E1G-ovalbumin conjugate. FIG. 5 is a standard curve for PdG where the strips have been sprayed with PdG-BSA as the capture material. The data given by the half strips (lacking a conjugate pad) are plotted against the level of E1G or PdG metabolite introduced (in units of amount per 24 hr) to show the relationship of the signal given by the strips to the standard concentrations. That is, these are calibration curves obtained with the strips from which the urinary concentrations can be read. The sensitivity of both curves is sufficient to measure menstrual cycle levels of E1G and PdG using timed urine simples. The menstrual cycle profile of E1G and PdG excretion rates obtained in this way are given in FIG. 6.

### Example 5

### Human Menstrual Cycle Profile for E1G and PdG

A human menstrual cycle profile is illustrated in FIG. 6. A menstrual cycle was then analysed by the strips with reading of the colour intensity to give the following menstrual cycle profile for E1G and PdG. The PdG data were only collected once the E1G peak was detected. For this cycle the first statistically significant E1G rise was on day 11, the E1G peak day was cycle day 15 and the beginning of the post-ovulatory infertility was cycle day 19 giving a fertile period of 8 days.

### Example 6

### Bovine Fertility Measurements

As with human data, corresponding standard curves were obtained with urine samples from dairy cows (FIGS. 7A and 7B). The E1G and PdG data were obtained with ELISA assays. FIG. 7A illustrates a standard curve for E1G and FIG. 7B illustrates a standard curve for PdG.

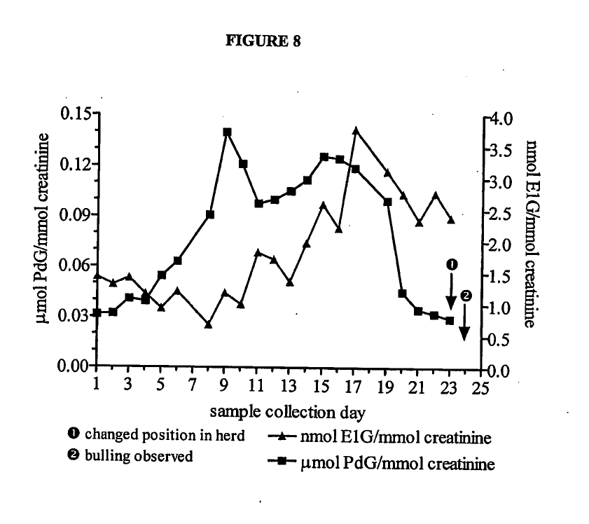

Bovine Daily E1G and PdG Excretion Rate Profiles from a cow are illustrated in FIG. 8. Daily data were obtained from cow 68 and the figure shows the E1G and PdG excretion for this period. The PdG data represent the corpus luteum from the previous cycle. This shows a marked decline 4 days before the next bulling. The E1G excretion rate peaks at about the time that the demise of the corpus luteum commences so both signals occur about 4 days before the next bulling in this cycle for this cow.

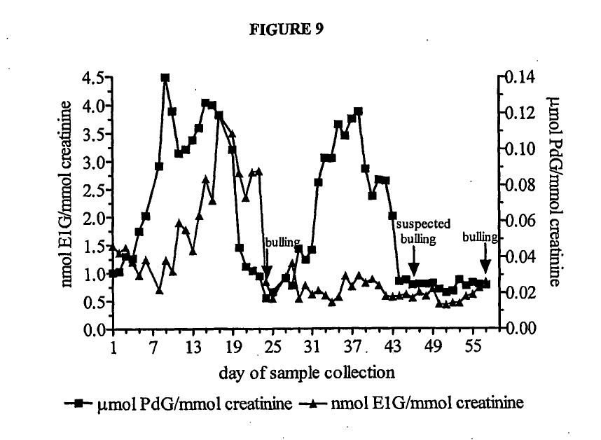

FIG. 9 shows two consecutive cycles for cow 68, the second being anovular. The first cycle is the same as the one above. These cycles are all corrected for variations in urine volume by use of creatinine excretion. Clearly there is no significant E1G excretion in the second cycle and further monitoring showed no PdG rise from days 45-59 in confirmation of its anovular nature. FIG. 9 shows the efficacy of the data obtained from urinary E1G and PdG data when corrected for urine volume by either creatinine or specific gravity. In FIG. 9 the second corpus luteum (shown by the rise in PdG level between days 29-45) results from the previous increase in E1G which indicates the presence of a dominant follicle. The first PdG peak (days 7-23) arises from the previous follicle which must have been present before the monitoring began. Note that where the third cycle was expected no PdG rise is evident. Also no E1G rise was evident in the second cycle. Thus this cow, just as some woman do, did not ovulate in the third cycle.

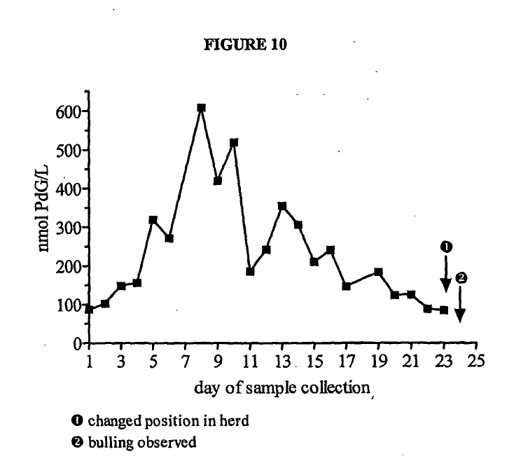

The presumed onset of the next estrous is shown by the very sharp decrease in PdG from days 22-25 and days 41-45. Thus this parameter is useful in predicting next oestrous but in some cycles this rapid decline in PdG will not be followed by ovulation. The lack of a PdG rise after day 45 reveals this fact. If correction for urine volume is not made the raw concentration data for PdG in the first cycle does not allow prediction of estrous (FIG. 10). It is apparent that the next oestrus could not be predicted from these PdG values from day 11 to day 25. The benefit of correcting for variation in urine volume and calculating a parameter approximating the excretion rate are obvious also in FIG. 13 for cow 228. For this cow, the next estrous may be indicated from the drop in the raw concentration data but it is distinctly more obvious after correction with creatinine.

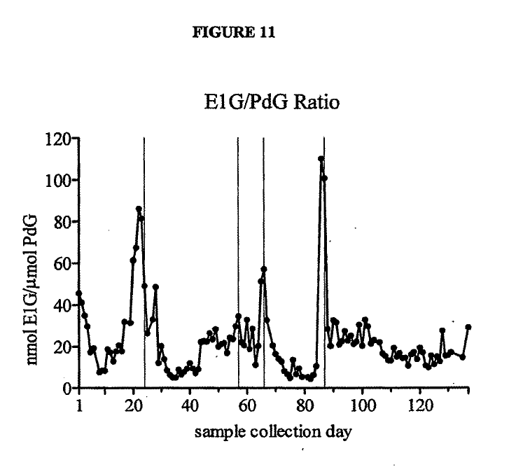

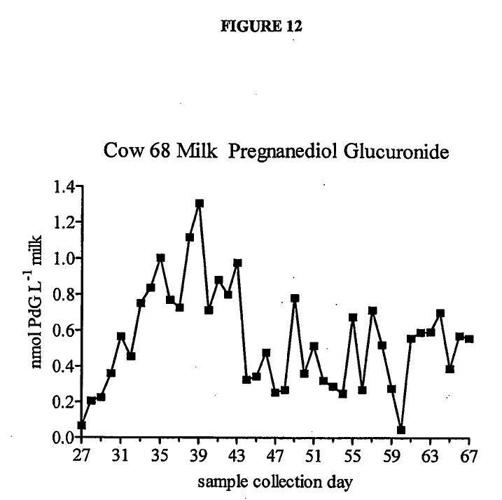

An alternative procedure for the bovine is to measure both E1G and PdG in the same urine sample with the strips and calculate the E1G/PdG ratio in which case the effect of variation in urine volume may disappear. The effect of this ratio and its relationship to the bulling behavior of the animal is shown in FIG. 11. The dotted lines indicate the bulling behavior as noted by the farmer. There is good agreement between the E1G/PdG peaks and bulling. However, note that in the second cycle where the hormonal data showed anovulation (days 25-60) no bulling was predicted from the ratio but the animal still showed bulling behavior (false oestrous). Pregnanediol glucuronide was also detectable in milk as shown in FIG. 12 for cow 68 and mirrored the urinary data. The levels were however much lower and no correction was made for variations in milk volume.

Finally the effect of using specific gravity as a basis for diluting the urine sample before measurement with half strips using latex bead conjugates is seen in FIG. 14. FIG. 14 shows that dilution with specific gravity gives a profile similar to that obtained with urines diluted to 150 ml/hr of collection. The profiles are similar except for the highest values which are read from the standard curve near its limit of accuracy on these standard curves.

In one experiment performed (data not shown), four cows were studied and 3 of the 4 cows became pregnant upon mating, which was detected by elevated levels of PdG after 21 days. Thus, accurate detection, monitoring, and modulation of the animal estrous/ovulation cycles can increase the effectiveness of reproductive management.

### Example 7

### Volume Adjustments

PdG Profile with Creatinine-Based Volume Adjustments

To correct for urine volume fluctuations it is necessary to make a measurement from which the urine is normalized. In one embodiment this is achieved by collecting all of the urine over a fixed time period and diluting to a constant volume so that all urines have the same total volume per hour of collection. For animals this is not possible. Hence in these cases a measurement must be made by which the hormone concentration data may be normalized by comparison to the amount of creatinine in the urine. This can be measured by the Jaffe reaction and the hormone concentration for each day divided by the amount of creatinine in the urine. FIG. 13 shows the smoothing effect on the PdG profile by such correction. Although the general profile is recognizable for this cow in the absence of urine volume correction it is clearly better with creatinine correction. A nearly 10 fold decrease in PdG/creatinine indicates the expected commencement of the next oestrous which occurred around day 23 as shown by the subsequent rise in PdG excretion.

PdG Profile with Specific-Gravity-Based Volume Correction

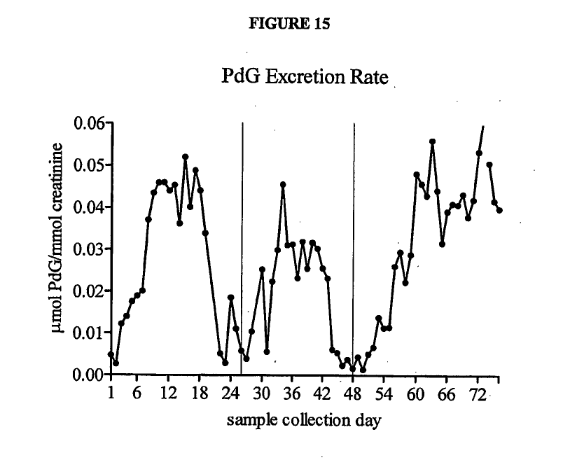

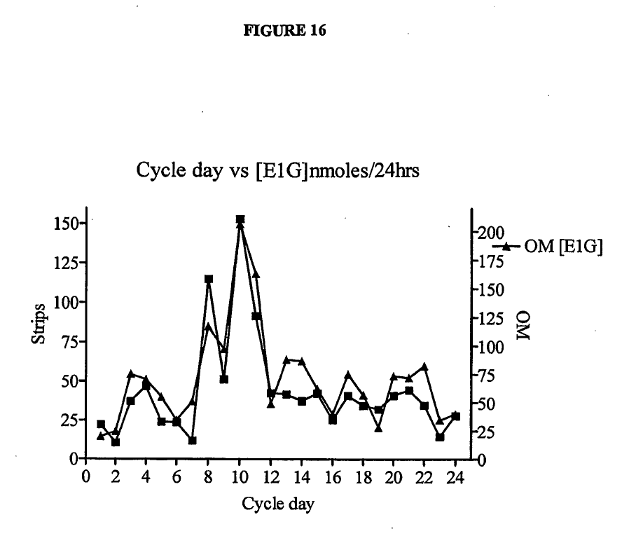

An alternative is to use specific gravity to make the correction. For example if the average specific gravity of a urine sample diluted to 150 ml/h is known then a dilution factor can be worked out for any urine sample on the basis of its specific gravity. This was done for a menstrual cycle and the PdG excretion rates determined on this basis compared with the usual time diluted samples. The results are shown in FIG. 14. FIG. 15 shows successive cycle of the pdG rise and fall and in the third cycle the cow was mated. The PdG rose as expected but did not fall again after 21-24 days indicating that cow (No. 228) was pregnant. This was later confirmed by veterinary diagnosis. FIG. 16 shows similarity in the excretion rate profiles for E1G and PdG between measurements obtained by the half strips method and the measurements obtained by the Ovarian Monitor method for the same urine samples. The agreement between the Ovarian Monitor data and strip data for the determination of MG excretion rates was indicative of the quantitative equivalence between the home-use strip and that of the Ovarian Monitor.

### Example 8

### Preparation of Conjugated Paramagnetic Particles

E1G was synthesised essentially according to the scheme of Bollenback et al., J of American Chemical Society 77:3310-3315 (1955) and Conrow & Bernstein, J of Organic Chemistry 36:863-70 (1971) and was used as a reagent for making the BSA-E1G capture material and for the E1G standards.

PdG purchased from the Sigma Chemical Company (Cat. No. P-3635) was used as a reagent for making the BSA-PdG capture material and for the E1G standards.

### E1G Paramagnetic Particle (PMP) Conjugation Protocol

A 10% suspension (10 mg, 100 μL) of carboxyl-modified magnetic latex particles (PMP) (R00-39 Estapor, Merck) diluted in 0.1 M MES, pH 7.0 buffer (900 μL) was activated with 10 mg EDC (PMP:EDC 1:1) at room temperature for 15 minutes with constant shaking. The magnetic particles were then pulled down with a strong magnet and the supernatant discarded. The activated latex was then reacted with 1.0 mL of a 1.0 mg/mL solution of mouse monoclonal E1G antibody (clone 2, IgG₂ₐ κ, purified by protein A affinity chromatography dialysed against phosphate buffered saline and freeze dried, Canterbury Health Laboratories, Christchurch, New Zealand) in 0.1 M borate buffer, pH 8.2. The mixture was vortexed and sonicated for 2 minutes to mix well, and then shaken at room temperature for 3 hours. The latex was blocked with 50 μL of 10% BSA in distilled water by shaking at room temperature for 30 minutes. The conjugated magnetic particles were then pulled down again with a strong magnet and the supernatant discarded. The pellet was resuspended into 1.0 mL of conjugate diluent to give a 10 mg/mL latex conjugate.

The conjugate diluent consisted of 10 mM borate buffer, 0.25% dextran, 0.25% Tween20, 1.0% BSA, 2 mM EDTA tetrasodium salt, 0.15% PEG (MW 10,000), 20% sucrose, 5% trehalose, 0.095% azide, pH 7.8.

### PdG Paramagnetic Particle Conjugation Protocol

This was carried exactly as for the E1G PMP conjugate but with the E1G antibody replaced with mouse monoclonal PdG antibody (clone 1 IgG_{2b} κ purified by protein A affinity chromatography, dialysed against water and freeze dried, Canterbury Health Laboratories, Christchurch, New Zealand).

E1G Capture Material

The E1G capture material was a BSA-E1G conjugate synthesised by the active ester method. The active ester reagent was made using a 1:3:1 ratio of DCC:NHS:E1G in freshly distilled DMF under dry conditions. 1.2 M stock solutions of NHS and DCC were prepared in DMF. 118 μL of the NHS stock solution was added to 46.5 moles of E1G dissolved in 41 μL of DMF followed by the addition of 41 μL of the DCC stock solution. After 2 hours the active ester reagent was added dropwise with gentle stirring to 43 mg of BSA dissolved in 2 mL 1% NaHCO₃ at 4° C. overnight to give an E1G active ester:BSA ratio of 72:1. The mixture was then dialysed over 24 hr (3×1 L) into 30 mM ammonium acetate buffer, with 0.02% sodium azide, pH 5.73 for mass spectral analysis, and then centrifuged at 2500 G for 5 minutes and the resultant clear supernatant removed from the white precipitate. The sample (2.5 mL) was then passed through a PD10 column (Cat. No. 17-0851-01, Pharmacia) pre-equilibrated with the dialysis buffer. Collection of eluent (3.5 mL) began when all the sample had run into the column. The final protein concentration was 8.4 mg/mL (Coomassie protein assay).

[BSA—fraction V, IgG free, fatty acid poor, Gibco, Cat. #30036-578]

### PdG Capture Material

The PdG capture material was a BSA-PdG conjugate synthesised according to the same protocol as that given for E1G and had a final concentration of 7.6 mg/mL.

### Example 9

### Nitrocellulose Membrane Preparation for the E1G Assay

The BSA-E1G capture material was dialysed into 10 mM phosphate buffer, pH 7.4 and diluted using the same buffer to 2 mg/mL prior to spraying. FF60 nitrocellulose membrane (35 mm, Schleicher & Schuell/Whatman) was sprayed with BSA-E1G as the test line and 0.5 mg/mL goat anti-mouse IgG (DCN, CA, USA) in the same buffer as the control line. The test line and control line were sprayed 6 mm apart, with the test line 12 mm from the bottom of the membrane using a Biojet dispensor (XYZ3050, Biodot, CA, USA). The membranes were striped at a rate of 75 μL/cm and dried at 37° C. for 2 hours in a forced air convection oven.

### Nitrocellulose Membrane Preparation for the PdG Assay

This was performed using the identical procedure to above, but with the BSA-E1G replaced with BSA-PdG capture material.

### Example 10

### E1G PMP Conjugate Pad Preparation

Glass fibre conjugate pad (9 mm×300 mm, Cat. No. GFCP203000, Millipore) was pretreated by soaking in 10 mM borate buffer, pH 8.0 containing 0.5% casein, 0.1% Tween 20 and 0.2% tauranol and 0.0.5% azide, for 30 minutes at room temperature. The pads were then placed on a paper towel, and dried at 37° C. overnight in a forced air convection oven. The treated pads were stored in a sealed foil pouch with desiccant. The E1G PMP conjugate was prepared for spraying by diluting in conjugate diluent (see conjugation protocol) to a concentration of 1.5 mg/mL, briefly vortexing and then sonicating in a water bath for 2 minutes. The conjugate, was then sprayed onto the dried pads at a rate of 12 μL/cm using an Airjet dispensor Airjet dispenser (Biodot, CA, USA). Sprayed pads were dried at 37° C. for 2 hours in a forced air convection oven and stored in sealed foil pouches with desiccant.

### E1G PMP Conjugate Pad Preparation

This was performed using the identical procedure to above, but with the E1G PMP replaced with PdG PMP.

### Sample Pad Preparation

CO48 cellulose sample pad (14 mm×230 mm Millipore) were pre-treated by soaking for 30 minutes at room temperature in sample pad buffer (0.2 M Tris, 4.0% Tween20, 0.05% sodium azide, pH 7.6). Excess buffer was removed by blotting the pads with absorbent paper towels and then the pads were dried for 5 hours at 37° C. in a forced air convection oven and stored in sealed foil pouches with desiccant.

### Test Strip Lamination Cutting and Assembly

The nitrocellulose membrane was laminated onto the middle portion of the backing card (Magna BioSciences, CA, USA) with the control line towards the top, followed by the 470 cellulose wick pad (18 mm×300 mm, Schliecher & Schuell/Whatman) to the top edge of the backing card giving a final overlap of 1.5 mm with the membrane. The PMP conjugate was laminated to overlap with the bottom edge of the membrane by 2 mm, followed by the lamination of the sample pad with the bottom edge of the backing card to give a final overlap with the PMP conjugate pad of 1-2 mm. The assembled cards were cut to 7.62 mm with the pin hole in the centre of the slicer (Magna BioSciences, CA, USA). The strips were placed in their cassettes (Magna BioSciences) and stored in sealed foil pouches with desiccant.

### Example 11

### E1G Standard Curve and E1G Menstrual Cycle Excretion Rates

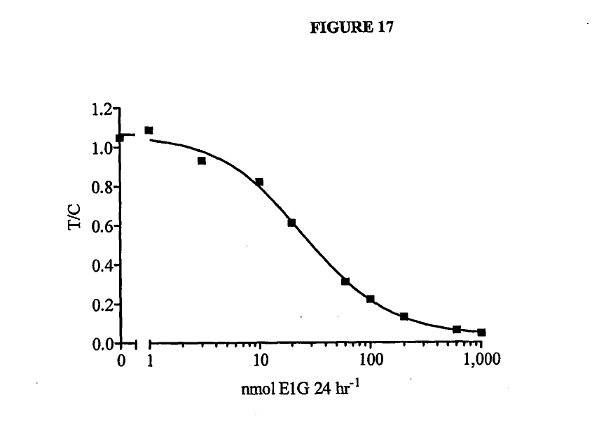

Blank urine for preparing the standards was obtained from a 4½ year old female child and time diluted to an excretion rate equivalent to 150 mL/hr. Standards covering the range 1-1000 nmol/24 hr (or 0.124-124 ng/mL) were prepared by serial dilution in the time diluted blank urine and then diluted a further ½ with water. The standard curve was performed by adding 140 μL of standard to the application well of the cassette and reading the control and test line MAR values after 15 minutes with a Magna BioSciences magnetic assay reader. The standard curve was generated by dividing by the MAR readings for the test line with the control line (T/C). See Table 2 and FIG. 17 for the resultant single point standard curve data and fit.

TABLE 2
E1G MAR Standard Curve

| [E1G] nmol/24 hr | [E1G] ng/24 hr | T/C |
|---|---|---|
| 0 | 0.000 | 1.049 |
| 1 | 0.124 | 1.088 |
| 3 | 0.372 | 0.931 |
| 10 | 1.240 | 0.821 |
| 20 | 2.480 | 0.611 |
| 60 | 7.440 | 0.310 |
| 100 | 12.400 | 0.220 |
| 200 | 24.800 | 0.130 |
| 600 | 74.400 | 0.063 |
| 1000 | 124.000 | 0.047 |

E1G daily excretion rates over a menstrual cycle were measured from urine samples provided by a 25 year old woman. Overnight urine samples were collected daily over a recorded time period and then time-diluted with tap water to 150 mL/hr. The time-diluted urine samples were then each diluted a further ½ before addition of 140 μL to the BIG cassette. The MAR results were expressed as T/C and the E1G excretion rate (nmol/24 hr) calculated from the corresponding standard curve. The cycle samples were also analysed by the Ovarian Monitor E1G assay system (Blackwell L. F., et al., Steroids 68:465-476 (2003) using the same time-diluted urines but without the extra ½ dilution. Both sets of data were collected in duplicate and expressed as averages. See Table 3 for the comparison of the E1G average excretion rates as measured by the two assay systems (NB: there was no sample collection for cycle days 11 and 19).

TABLE 3
Menstrual Cycle E1G Excretion Rates as Measured
by the MAR System and the Ovarian Monitor

[E1G] nmol/24 hr

| cycle day | MAR (T/C) | OM |
|---|---|---|
| 2 | 6 | 180 |
| 3 | 3 | 129 |
| 4 | 14 | 135 |
| 5 | 10 | 137 |
| 6 | 2 | 179 |
| 7 | 91 | 232 |
| 8 | 56 | 242 |
| 9 | 104 | 345 |
| 10 | 283 | 559 |
| 11 | | |
| 12 | 230 | 472 |
| 13 | 203 | 560 |
| 14 | 72 | 238 |
| 15 | 53 | 318 |
| 16 | 26 | 224 |
| 17 | 87 | 346 |
| 18 | 42 | 331 |
| 19 | | |
| 20 | 38 | 391 |
| 21 | 169 | 336 |
| 22 | 81 | 320 |
| 23 | 43 | 259 |
| 24 | 24 | 230 |
| 25 | 37 | 221 |
| 26 | 74 | 153 |

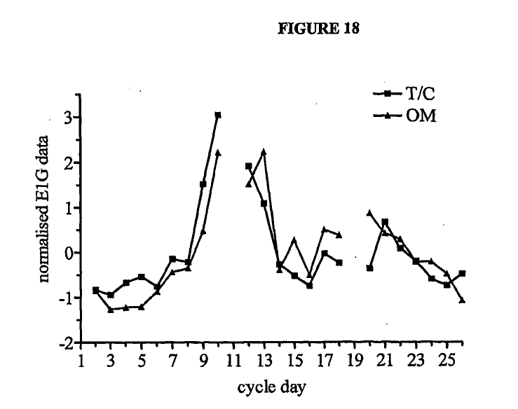

Since the Ovarian Monitor derived E1G excretion rates were higher than the MAR derived values, the cycles were normalised to allow the hormone excretion rate patterns obtained with the two systems to be compared. This required for each method for the mean MG excretion rate data for the full cycle to be subtracted off each individual cycle day's excretion rate, and then dividing each of these values by the standard deviation of the mean differences (standard normal variant transformation). The normalised data are shown in FIG. 18.

The normalised patterns show excellent agreement. The MAR E1G excretion rates carry the same information regarding follicle growth and maturation as does the validated reference assay (Blackwell L. F., et al., Steroids 68:465-476 (2003). The day of the first sustained rise in urinary E1G excretion levels has been defined as the first day of fertility. This is because E1G is a biomarker for the biologically active estrogen, estradiol, and an increase represents the selection of a potentially ovulatory follicle—the source of the estradiol. Both the MAR and Ovarian Monitor assays gave a significant rise greater than experimental error on day 7. A simple calculation gives the baseline mean for the Monitor data as 155 nmol/24 hr with a standard deviation of 25.3. The calculation is performed by defining a baseline period and taking the mean of the highest and lowest excretion rate over this period. For example, in this cycle the baseline consists of the cycle days 2-6. The mean of 180 and 129 nmol/24/hr approximates to the mean for the excretion rate for the baseline period. An approximation to the standard deviation is given by the difference between the mean and the highest excretion rate in the baseline period. The first day the excretion rate exceeds the mean plus 2 standard deviations (205 nmol/24 hr) is day 7 when the value is 232 nmol/24 hr (Table 2). For the MAR data the calculation gives the mean as 8.8 nmol/24 hr (SD=5.6) thus the threshold value is 20 nmol/24 hr which is clearly exceeded on day 7 by an E1G excretion rate of 91.3 nmol/24 hr showing the presence of a growing follicle in the ovary. The sample for day 11 is missing and this is most likely the peak day of E1G excretion by both assays.

### Example 12

### PdG Standard Curve and PdG Menstrual Cycle Excretion Rates

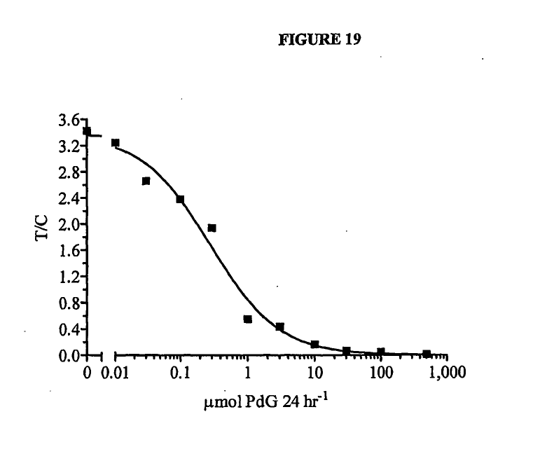

Blank urine for preparing the standards was obtained from a 4½ year old female child and time diluted to an excretion rate equivalent to 150 mL/hr. Standards covering the range 0.01-500 μmol/24 hr (or 1.38-69,000 ng/mL) were prepared by serial dilution in the time diluted blank urine and then diluted a further ⅕ with water. The standard curve was performed by adding 140 μL of standard to the application well of the cassette and reading the control and test line MAR values after 15 minutes with a Magna BioSciences magnetic assay reader. The standard curve was generated by dividing by the MAR readings for the test line with the control line (T/C). See Table 4 and FIG. 19 for the resultant single point standard curve data and fit.

TABLE 4
PdG MAR Standard Curve

| [PdG] μmol/24 hr | [PdG] ng/mL | T/C |
|---|---|---|
| 0.00 | 0.00 | 3.43 |
| 0.01 | 1.38 | 3.25 |
| 0.03 | 4.14 | 2.66 |
| 0.10 | 13.80 | 2.38 |
| 0.30 | 41.40 | 1.94 |
| 1.00 | 137.90 | 0.55 |
| 3.00 | 413.70 | 0.43 |
| 10.00 | 1379.00 | 0.17 |
| 30.00 | 4137.00 | 0.07 |
| 100.00 | 13800.00 | 0.05 |
| 500.00 | 69000.00 | 0.01 |

PdG daily excretion rates over days 7-26 of a 26 day menstrual cycle were measured from urine samples provided by a 33 year old woman. Day time urine samples were collected daily over a recorded time period and then time-diluted with tap water to 150 mL/hr. The time-diluted urine samples were then each diluted a further ⅕ before addition of 140 μL to the PdG cassette. The MAR results were expressed as T/C and the PdG excretion rate (nmol/24 hr) calculated from the corresponding standard curve. The cycle samples were also analysed by the Ovarian Monitor E1G assay system (Blackwell L. F., et al., Steroids 68:465-476 (2003) using the same time-diluted urines but without the extra ½ dilution and also by ELISA with an extra 1/50 dilution using a modified procedure of Henderson K. M., et al., Clin Chim Acta. December 29; 243(2):191-203 (1995). All sets of data were collected in duplicate and expressed as averages, with the exception of 3 days MAR data for which a T/C value could not be calculated due to a failure of the reader to measure the control line—for these days the results are necessarily in single point. Table 5 for the comparison of the PdG average excretion rates as measured by the three different assay systems (NB: there was no sample collection over the first 6 days of the cycle).

TABLE 5
Menstrual Cycle PdG Excretion Rates as measured
by the MAR System, the Ovarian Monitor and ELISA

| | [PdG] μmol/24 hr | | |
|---|---|---|---|
| Cycle Day | MAR (T/C) | OM | ELISA |
| 7 | 0.8 | 1.2 | 0.7 |
| 8 | 1.2 | 1.9 | 0.6 |
| 9 | 1.0 | 2.5 | 0.7 |
| 10 | 0.8 | 2.9 | 0.8 |
| 11 | 0.8 | 2.7 | 0.7 |
| 12 | 0.3 | 3.2 | 0.9 |
| 13 | 1.2 | 3.1 | 1.3 |
| 14 | 1.5 | 3.4 | 1.7 |
| 15 | 0.8 | 2.4 | 1.8 |
| 16 | 2.6 | 6.8 | 5.3 |
| 17 | 3.4 | 6.7 | 5.1 |
| 18 | 4.4 | 8.8 | 7.3 |
| 19 | 4.6 | 10.1 | 8.0 |
| 20 | 6.0 | 13.3 | 10.4 |
| 21 | 6.1 | 10.3 | 8.1 |
| 22 | 6.5 | 8.9 | 7.8 |
| 23 | 5.0 | 17.4 | 10.6 |
| 24 | 5.4 | 10.9 | 7.9 |
| 25 | 3.9 | 4.4 | 4.5 |
| 26 | 1.8 | 3.2 | 2.2 |

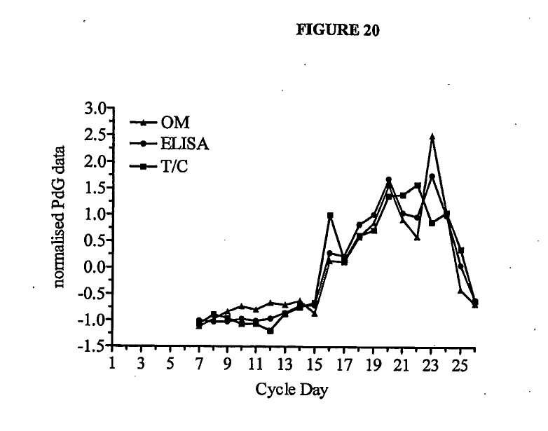

Since the Ovarian Monitor derived PdG excretion rates were higher than the MAR derived values, the cycles were normalised to allow the hormone excretion rate patterns obtained with the three systems to be compared. This was performed using a standard normal variate transformation as described for the previous E1G data. The normalised data are shown in FIG. 20.

Although the absolute PdG excretion rates obtained with the MAR cassettes are lower, the profiles are identical within the experimental errors of the methods. The post-ovulatory PdG rise is clear on day 16 in all cases. The Ovarian Monitor PdG fertility markers are set as follows: the PdG threshold that marks the end of fertility is set at ≧6.3 μmol/24 hr (Blackwell, L. F., et al. Steroids, 63, 5. (1998), the biochemical proof of ovulation marker is set at 9 μmol/24 hr and the marker for proof of an adequate corpus luteum (capable of supporting pregnancy) is set at 13 μmol/24 hr. Normalising for the MAR system this translated into excretion rate readings of 2.2, 4.6 and 6 μmol PdG/24 hr respectively. For both the Ovarian Monitor and the MAR system these 3 thresholds were reached on days 16, 19 and 20 respectively for both systems e.g. once the data was normalised the two systems were shown to give the same days for all the key PdG fertility markers.

### Example 13

Application to Menstrual Cycle Data

### E1G Excretion Rates as a Marker for the Beginning of the Fertile Period

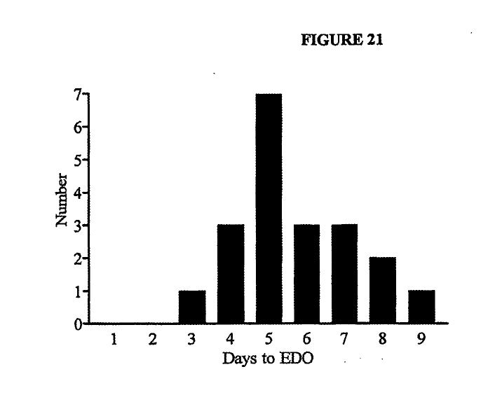

The data confirm that the MAR test results mimic the reference tests (either the Ovarian Monitor or the in-house ELISA assays). Thus the presence of a growing follicle and its growth to maturity can be followed by the rising values of the E1G excretion rate and ovulation and the quality of the corpus luteum can be monitored by the magnitude of the PdG excretion rates. For E1G excretion rates obtained with time diluted urine samples and using the day of the first statistically significant rise above the preceding baseline as determined by the Trigg's tracking signal for the beginning of the fertile window (Blackwell, L. F. and Brown J. B. Steroids, 57, 554 (1992) to the day following the mid-cycle peak in E1G excretion, the prospective warning of ovulation given for 20 cycles from published RIA data (Blackwell L. F., et al., Steroids 68:465-476 (2003) was as shown in FIG. 5. This is the warning to be expected from monitoring of E1G excretion rates using time diluted urine samples and gives a mean warning of 5.7 days (N=20) which is sufficiently early to avoid conception except possibly for the longest sperm survival times (>6 days) (Austin C R, J Reprod Fertil Suppl 22:75-89 (1975). It has been estimated that only 6% of pregnancies are due to sperm survival times greater than 3 days (Wilcox et al., New England J of Medicine 333:1517-21 (1995). Furthermore, the women with cycles with a short warning period of ovulation based on E1G are more likely to have had adequate warning of the beginning of their fertile phase than if this was the days of warning given for a woman with a cycle with a longer E1G rise. This is because these cycles with a short warning of ovulation based on E1G are less likely to be support extended sperm survival times, as one of the functions of estrogen is the stimulation of fertile mucus, without which sperm survival is exceedingly short. The First Rise Day to Estimated Day of Ovulation is shown in FIG. 21. A larger study (Blackwell, L. F. and Brown J. B. Steroids, 57, 554 (1992) has shown that detection of the first statistically significant rise in estrogen excretion rates gives a warning of impending ovulation of 6.5±1.4 days. These figures will apply to the E1G excretion rates determined by the PMP cassette assays.

### PdG Excretion Rates as a Marker for the End of the Fertile Period

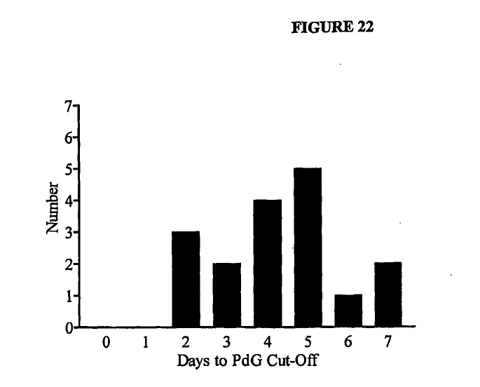

It is well accepted that any marker of the end of fertility must not delinate it to occur before the day of the mid-cycle E1G (or LH) peak. As shown in Table 6, the earliest day that the PdG threshold value was exceeded came 2 days after the mid-cycle E1G peak excretion rate day for 20 cycles analysed by RIA (Blackwell L. F., et al., Steroids 68:465-476 (2003). The use of a PdG threshold has an additional advantage over other markers for defining the end of the fertile period that extends past its temporal relationship with ovulation. A high circulating level of progesterone, the source of urinary PdG, is associated with infertility through the stimulation of the production of infertile mucus. In fact, this is one of the main means of action of the progesterone only mini-pill (ovulation has been estimated to only be prevented in 50% of cycles). In addition, high circulating levels of pregnanediol (an intermediate between progesterone and PdG) during the pre-ovulatory phase are known to be associated with infertility and high spontaneous abortion rates. Finally, the fact that throughout an estimated total of 4,000 women years of experience with the Ovarian Monitor, no pregnancies have resulted from the use of this threshold marker provides the strongest proof as to its validity for determining the end of the fertile period (Blackwell, L. F., et al. Steroids, 63, 5. (1998). The number of days from E1G Peak to PdG Cut-Off Day is illustrated in FIG. 22.

Assuming that the reproducibility of the MAR cassettes can match the Ovarian Monitor, the use of time diluted urines (e.g. correction of samples for hydration status) with the MAR tests will give the same warning as exhibited by the published PdG excretion rate data.

### Antibodies and Position of the MAR E1G and PdG Standard Curves

For best results it is essential that the measurements be made in the working range of the standard curve. For example the E1G standard curve shown in FIG. 17 is optimal over the excretion rate range of about 1 to 100 nmol/24 hr when the standards and samples are diluted 1 in 2. However, since the normal range of menstrual cycle E1G excretion rates is from about 8 to 465 nmol/24 hr, even with an extra dilution of ½ the signal from the test will encompass only the lower portion of the standard curve. Calculations show, and experiments confirm, that an extra 1/5 dilution of the E1G standards and the time-diluted samples will give the optimum responses from the MAR cassettes for the measurement of menstrual cycle urines. In other words the current cassettes are operating best over the range of 2 to 40 nmol/24 hr.

Similarly with the current antibodies used in the MAR PdG assay system, the optimum PdG standard curve covers the excretion rate range from 0.002 to 10 μmol/24 hr.

Thus the current antibodies require a 1/5 dilution of the time-diluted samples for the E1G test and a 1/25 dilution for the PdG test.

To avoid these dilutions in normal cycles, new antibodies need to be raised with characteristics such that no dilution of the time-diluted samples is necessary for normal menstrual cycles. This can be achieved by screening of clones produced by standard hybridoma techniques or other modern antibody generation techniques for less sensitive antibodies. Traditionally producers of commercial antibodies select the clones that give the lowest possible detection limit. For the present purposes clones will be selected with less sensitive characteristics, but which are ideal for the measurement of E1G and PdG excretion rates in time diluted, and possibly undiluted, urine samples.

Also, alternative clones originally generated when selecting for the existing E1G and PdG monoclonal antibodies might be re-examined for more favorable characteristics. As mentioned above if a clone does not produce a high titre, high affinity antibody (with a low detection limit) it is usually not investigated further for ELISA assays where maximum sensitivity is almost always sought.

An alternative solution is to find a commercial antibody-capture material binding couple that gives a less sensitive standard curve by using a capture material made with the appropriate steroid analogue and a linker to the capture protein. This involves screening of a variety of steroid derivatives, including steroid linker conjugates, against available monoclonal antibodies. There is some evidence that standard curves of differing sensitivity can result from such screenings even with the same antibody.

For example, a monoclonal antibody (Wallaceville Animal Research Centre (WARC), Upper Hutt, New Zealand) when used with an E1G glucose oxidase conjugate gave a standard curve with a mid-point of 5000 nmol/24 hr which was too insensitive to be used with pre-diluted urine samples. This assay system was 250 times less sensitive than the PMP cassette assay described in this patent. When an estrone BSA conjugate with an 8-carbon linker was used with this antibody the standard curve was even less sensitive. On the other hand the same antibody could be used with 6-ketoestrone carboxymethyl-oxime and estrone hemisuccinate conjugates to measure menstrual cycle excretion rates of E1G in urine samples (Henderson K. M., et al., Clin Chim Acta. December 29; 243(2):191-203 (1995). Hence choice of the correct linker can give a standard curve of the desired sensitivity.

Binding studies using surface plasmon resonance and a WARC monoclonal antibody against E1G showed that the binding was a function of the capture protein and the linker used.

TABLE 6
BSA Conjugate Binding as measured
by Surface Plasmon Resonance

| | Substitution | RU Binding | Concentration |
|---|---|---|---|
| Conjugate | Level | Units | (μg/mL) |
| BSA-E1G | 28-35 | 90 | 62.5 |
| BSA-C3C5-E1G | 13-20 | 163 | 62.5 |
| BSA-C5-E1G | 8-12 | 204 | 62.5 |

The BSA E1G conjugate linked by a 6-aminohexanoic acid moiety (BSA-05-E1G) gave the best binding since it had the least number of E1G molecules/protein and the least sensitive standard curves. A number of conjugates with lesser binding would be expected to give more sensitive standard curves.

### Accuracy of Test

A common problem with lateral flow or dipstick assays is the consistency of release of the coloured reagent from the conjugate pads. The precision of a quantitative test is highly dependent on this aspect. Factors such as variability of conjugate application, re-hydration, flow rate and the number of particles released all combine to lower the precision. Hence, in the next phase, the conjugate pad will be removed from the assay system and replaced by a lyophilised sample in a tube or syringe. Thus the conjugate can then be rehydrated and resuspended in the presence of the urine sample and then applied to the sample pad of the cassette. In this way precision of the tests should be dramatically improved. The acquisition of excretion rates, which parallel the changes in values as a function of ovarian activity delivered by reference assays as shown in FIGS. 18 and 8, and that have a coefficient of variation of less than 10% is unique. These data give access to the vast body of literature and other data that exists on the application of E1G and PdG excretion rates to reproductive biology of mammals including humans in all of its aspects (Baird, et al., Fertility and Sterility 71(1):40-49 (1999), as shown for example in FIGS. 19 and 20.

By avoiding binding of the conjugates to hydrophobic materials and using a syringe or related device it is possible to envisage a device that will allow a woman to collect a time-diluted urine sample and then automatically take up an aliquot of the sample and apply a set volume accurately to the cassette.

### Calculation of Results

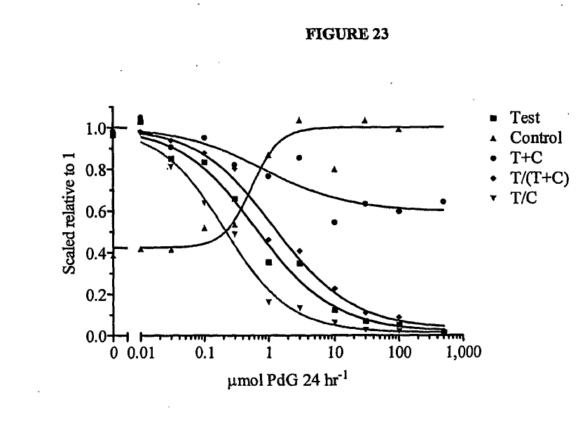

The output from the cassette tests consists of the magnetic intensity of the bound paramagnetic particles localised on the test line and one or more control lines. The variability in conjugate pad release can be simply observed by repeat runs of a single hormone concentration where it displays itself either as general large strip to strip variation or high strip to strip repeatablility with the occasional extreme outlier. Both of these types of variation can be partly corrected for by expressing the data relative to the control line, either as a fraction (T/T+C) or as a ratio (T/C). Thus to avoid the standard curve exhibiting large deviations from the best fit line and single point anomalies, a simple plot of T versus log [hormone] has been replaced by the use of the control line corrected data. However, since C and (T+C) both show a dependence on the hormone concentration (e.g. exhibit a standard curve) there are significant differences between the standard curves plotted by the three methods (T, T/C or T/(T+C) versus log [hormone]. FIG. 23 illustrates the factors influencing the PdG MAR Standard Curve Correction Methods.

The standard curves are fitted by non-linear regression to the equation:

Y=Y_{∝}+[Y₀−Y_{∝}]/[1+10^{[[logEC50-X]*slope]}]

Where Y_{∝} is the lowest reading (at infinite standard excretion rate), Y₀ is the reading at zero excretion rate and EC₅₀ is the excretion rate at the mid-point of the standard curve.

As long as the E1G or PdG standards are diluted the same as the urine samples, the E1G or PdG excretion rate is determined simply by extrapolation from the appropriate standard curve in nmol/24 hr (E1G) or μmol/24 hr (PdG).

### Example 14

### Volume Adjustment Using Time-Diluted Urine Samples

Urine based quantitative assays typically need to address the discrepancy between analyte concentration in the urine and the rate of analyte excretion. Obviously the greater the rate of urine production per unit time, the more diluted an analyte will become, even if it is released into the bladder at a constant rate. One of the key advantages of the present method is that the excretion rates are determined on urine samples time-diluted to a constant excretion rate of 150 mL/1 hr of urine collection. Analysis of time diluted urine samples give the most accurate data possible, not only because this method corrects for variation in hydration status but also because any matrix effects between urine samples are also minimised (by making it more constant) (see Blackwell L. F., et al., Steroids 68:465-476 (2003).

Partial corrections can be made by collecting first morning void samples. This method is based on the premise that the rate of urine production is more constant over the night as the water intake and energy expenditure is most variable during the day. However it has been the experience of our lab that the range of urine production rate (mL/hr) in early morning urine samples over the duration of a menstural cycle is the order of a factor of 10. Another means of correction is to divide by the creatinine concentration. This method is based on the premise that creatinine excretion (which is related to muscle mass) is constant. However creatinine excretion is known to decrease with age and vary with nutritional status and is effected by moderate to heavy exercise.

The simplest procedure, particularly for use with infertility patients, is to collect a sample over a known time period (as described by Blackwell L. F., et al., Steroids 68:465-476 (2003) in a calibrated jug and dilute to 150 mL/hr. The collection time may be as short as 3 hours although the results are expressed as per 24 hours for convenience.

New methods based on chemical properties may be developed which allow correction for urine volume.

### Example 15

### Exemplary Applications of the Excretion Rates

Given accurate and reproducible PMP cassette assays a large number of applications of the E1G and PdG excretion rates are possible. We intend to develop protocols for using the E1G and PdG excretion rates in all clinical and home use aspects of fertility and infertility. The principles behind the protocols are:
The first statistically significant rise in E1G excretion rates indicate the presence of a growing follicle and hence the beginning of potential fertility
A rise in PdG excretion rates to exceed a threshold, such as 6.3 μmol/24 hr with the Ovarian Monitor, or its equivalent with the PMP cassette assays, indicates the end of fertility
A mid cycle peak in E1G excretion rate followed by a small rise in the rate of PdG excretion indicates ovulation and the most fertile time for intercourse to achieve a pregnancy
Biochemical proof of ovulation is provided by an increase in the PdG excretion rate above the Ovarian Monitor assay equivalent of 9 μmol/24 hr
An adequate corpus luteum is shown by an increase in the PdG excretion rate above the Ovarian Monitor assay equivalent of 13 μmol/24 hr.

### EXAMPLES

The following examples are representative of the way in which these test might be integrated into clinical and personal management of fertility and infertility. Most of the experience discussed has been obtained by home monitoring using the Ovarian Monitor.

However, given the substantial equivalence of the PMP cassette assays with the Ovarian Monitor data the protocols described are directly transferable to the PMP cassette assay data by adjustment of any thresholds into PMP equivalents.

### 1. Avoidance of Pregnancy—Normal Cycle

Urine samples collected and diluted according to time (150 mL/hr) for a minimum of three hours collection. The urines were analysed by the Ovarian Monitor using 504 of time-diluted urine for E1G and 10 μL of time-diluted urine for PdG. The results are reported as change in transmission units per unit time; ΔT/20 minutes for the E1G assay and ΔT/5 minutes for the PdG assay. This is a typical cycle determined at home by the subject herself. The E1G excretion rates were measured daily for the follicular phase of the cycle and when an E1G peak day was determined, measurement was changed to PdG.

TABLE 7
Ovarian Monitor Cycle Data as Collected for Pregnancy Avoidance

| | E1G | PdG |
|---|---|---|
| Cycle day | (ΔT/20 min) | (ΔT/5 min) |
| 5 | 53 | |
| 6 | 69 | |
| 7 | 67 | |
| 8 | 78 | |
| 9 | — | |
| 10 | 98 | |
| 11 | 85 | |
| 12 | 70 | |
| 13 | 126 | |
| 14 | 146 | |
| 15 | 163 | |
| 16 | 205 | |
| 17 | 161 | 119 |
| 18 | | 253 |
| 19 | | 289 |

The commencement of the fertile phase is obviously on day 13 when the E1G excretion rate increases above the previous baseline average. This indicates the expression of ovarian aromatase activity and shows that a dominant follicle is present in which the ovum is surrounded by an estrogenic milieu. This follicle will end its life either in ovulation or in atresia. The rise may be calculated by taking days 5-12 as baseline. The approximate mean is (53+98)/2 or 75.5 ([lowest+highest/2]). The approximate standard deviation is (98−75.5) or 22.5 (highest—mean). Twice SD is 45 therefore the threshold value is (75.5+45) or 120.5 (Mean+2SD). The first day to exceed this calculated excretion rate is day 13 in agreement with the visual assessment. The peak E1G day is day 16, which indicates day 17 as the most fertile day. The end of fertility is day 18 since the PdG value exceeds the PdG threshold value (180 ΔT/5 min—equivalent to 6.3 mmol/24 hr for this set of Ovarian Monitor PdG tubes).

Similar algorithms would apply to the MAR cassette data since the E1G and PdG profiles paralleled those given by the Ovarian Monitor (see FIGS. 8 and 20). Important values on the MAR cassette platform need to be finalised in pre-clinical studies.

The above data can be utilised to avoid conception by avoiding intercourse after day 12 since day 13 is day 1 of the fertile window (Blackwell, L. F. and Brown J. B. Steroids, 57, 554 (1992). Once the PdG value exceeds the cut-off intercourse (day 18 in the above example) may be resumed since the cycle is infertile until the next bleed. Examples of the use of this protocol may be found in Brown et al., American Journal of Obstetrics and Gynecology—Supplement 165:2008-11 (1991). Clearly the present invention lends itself to a similar usage.

2. Avoidance of Pregnancy when Navigating through the Continuum

The range of ovarian activities that may be experienced by a woman, from amenorrhoea to a fully fertile ovulatory cycle, is called the continuum (Brown, J B, Scientific Basis and Problems of natural Fertility Regulation, a meeting at the Pontcal Academy of Science in Room (1994). The progression up through the continuum is clearly observed from the year preceding menarche until approximately three years post-menarche, and also during the return to fertility postpartum, while regression back through the continuum is experienced as women approach the menopause. Other women included in the lower end of the continuum (e.g. with subfertile ovarian activity) include women with dysfunctional bleeding and women 0.15 returning to fertility after oral contraceptive use. Deficient ovarian activity is also common in professional athletes, and in other women involved in circumstances that are associated with extreme physical or mental stress or weight loss. Even within the 20-40 year age group, the incidence of fully ovulatory cycles in unstressed women is only 90%.

The changes in a woman's position in the continuum from cycle to cycle are unpredictable; some stages in the continuum may be skipped, and a fully ovulatory cycle can occur at any time. Confusion in symptoms caused by the temporary passing through the infertile region of the continuum is one of the main causes of unplanned pregnancies in NFP.

The problem with most natural family planning methods is that they are primarily suited only to women from the top end of the continuum e.g. women with regular ovulatory cycles who exhibit the classical patterns of fertility. When these methods do take into account the ‘other’ women with irregular cycles, the guidelines are usually associated with long and excessive periods of abstinence over periods of life or conditions that are often in fact associated with low fertility.

Many of the women who use the Ovarian Monitor for pregnancy avoidance do so because of irregular cycling and the associated problems the more traditional methods of natural family planning produce for them. The proportion of this subgroup who have already experienced an unplanned pregnancy is particularly high (50%) (Brown et al., American Journal of Obstetrics and Gynecology—Supplement 165:2008-11 (1991). For these women, the attraction of the Monitor is the security the method provides; the hormonal assays allow them to define precisely their periods of fertility and changing position within the continuum. The guidelines for the use of the Ovarian Monitor throughout the continuum are very straightforward. If the E1G levels rise a dominant follicle is present and fertility must be assumed. When the E1G levels fall, the cycle should be tested for ovulation with the PdG assay. Once the PdG threshold is reached infertility may be assumed with safety until the next menstrual bleed. The different positions within the continuum as determined by the Ovarian Monitor hormone assays are outlined below.

In the complete absence of ovarian activity, the E1G and PdG levels remain uniformly low and menstruation does not occur. Obviously this represents the lowest level of the continuum and is associated with absolute infertility. This situation may be permanent or temporary.

If E1G levels are observed to rise and fall between bleeding episodes but the PdG levels remain uniformly low, the cycle is anovulatory. Anovulatory cycles fall into two main categories. In the most common type, the E1G levels rise indicating that a follicle has developed, but the estrogen rise is insufficient to trigger the LH surge and the follicle dies by atresia, causing E1G levels to fall, and bleeding due to estrogen withdrawal. In other anovulatory cycles the E1G levels rise to reach a plateau and the values remain at this plateau for a variable period of time. The plateau levels of E1G are usually lower than for the usual pre-ovulatory E1G peak and bleeding eventually occurs as a breakthrough phenomenon e.g. the elevated levels of circulating estradiol over prolonged periods of time leads to extensive proliferation of the endometrial layer to such a level that it cannot be maintained.

The unruptured, luteinised follicle represents the next stage in the continuum. In these cycles the E1G levels rise and fall, but represent an estradiol level that is too low to induce a fully ovulatory LH peak. However it is sufficient to cause some LH mediated luteinisation of the follicle. Partial luteinisation results in a marginal elevation of PdG levels after the E1G fall, however their suboptimal levels provide proof that the follicle was never ovulatory. A cycle is defined as having a luteinised unruptured follicle if the PdG levels rise to between 4.5 to 6.3 μmole/24 hr for two or more days.

The fact that all of the anovulatory conditions described above, may be followed immediately by a fertile ovulatory response with or without an intervening bleed, demonstrates some of the difficulties women face when moving within the continuum.

Cycles with short or deficient luteal phases represent the next step up in the continuum. A deficient luteal phase is one in which the PdG values rise to exceed 5 μmol/24 hr, but do not reach the ovulatory threshold of 9 μmol/24 hr, and a short luteal phase is one in which the PdG values exceed 9 μmol/24 hr but the post-ovulatory phase lasts for 11 days or less. Although both cycles are associated with a normal follicular phase, the cycles are infertile as their luteal phases are incapable of supporting pregnancy.

The fertile ovulatory cycle represents the highest level in the continuum. The fertile cycle is characterised by a well defined E1G peak followed by ovulation and a luteal phase which surpasses a PdG excretion rate of 9 μmol/24 hr and lasts a minimum of 12 days. At these minimum levels conception rates are 25% per cycle. Higher E1G and PdG values (PdG 36 μmol/24 hr) are more common and are associated with conception rates of 70% per cycle (Brown, J B, Scientific Basis and Problems of natural Fertility Regulation, a meeting at the Pontifical Academy of Science in Room (1994). The administration of estrogen analogues and gonadotrophins further elevates fertility and increases the possibility of multiple pregnancy by inducing superovulation. Such treatments have provided a 47% conception rate in patients with long-standing infertility (Brown, J B, Scientific Basis and Problems of natural Fertility Regulation, a meeting at the Pontifical Academy of Science in Room, (1994).

3. Return to Fertility after Breast Feeding

This is a difficult time in family planning when hormonal contraception may be contra-indicated. Although it is known that the chances of conception are probably less than 2% when fully breast-feeding (Kennedy et al., Contraception 39:477-96 (1989) there is a period when fertility returns and the chances of conception increase. An ovulatory cycle may occur before the first post-partum menstrual bleed. Monitoring of ovarian hormones can guide a woman safely through this period of returning fertility. The following phases have been recognised based on the Melbourne experience (see Blackwell, L. F., et al. Steroids, 63, 5. (1998).

### Establishment of E1G Baseline:

This is done by testing daily, consecutive, urine samples for E1G over a period of 7 days. A contact person experienced in application of the Monitor data is notified of the results and the baseline E1G level is established for the woman. Each woman must be treated as an individual.

Use during 0-6 Months Post Partum:

The E1G excretion rate is checked twice per week. If the E1G excretion rate is below baseline levels then the woman is in an infertile phase for a variable number of days. If the E1G excretion rate is low, experience suggests that new follicle will not appear before 6-7 days have elapsed so a week's safety for unrestricted intercourse is indicated. If the E1G excretion rate is above the baseline level or there is a change in the basic infertile mucus pattern (BIP) as described in the Billings Ovulation method, daily E1G tests are continued. A contact person may be notified who will advise the client of a regime for further testing. If there is a previous history of returning to fertility earlier than 6 months then the E1G excretion rate should be checked twice a week from 0-2 months increasing to three times per week after that.

Use between 6-9 Months Post Partum:

The E1G excretion rate is checked every third day. If at, or below, the individual's, baseline value the woman is in an infertile phase but less free time is available for unrestricted intercourse. If the E1G excretion rate is above the baseline level or there is a change the BIP daily E1G tests are continued. A contact person will advise of further testing. (Possibly via internet or reader algorithms.)

Use from 9 Months to Weaning:

The E1G excretion rate is checked every second day. If at, or below, the baseline level the woman is in an infertile phase. If the E1G excretion rate is above the baseline or there is a change in the BIP, daily E1G testing is continued. A contact person will advise of a further testing regime

### Comments on Interpreting the Results

I Days of infertility: i) E1G excretion rate at or below a baseline value
ii) PdG values above the cut off or threshold value
   II Days of Fertility: All days with raised E1G excretion rates above the individual's baseline value and associated with low PdG excretion rates.

A rise in E1G excretion rate above the baseline indicates the beginning of the potentially fertile phase. The E1G values continue to rise for 3 to 7 days to reach a peak. They then fall abruptly. On the day of the fall, PdG measurements should be commenced. The PdG excretion rate on the day of the fall will be low but it will continue to rise and on the third day be approaching or will have passed the PdG cut off (or threshold) value. Once the cut-off has been reached, ovulation has already occurred and the fertile phase has ended. No further testing is required for the remainder of the cycle.

The E1G excretion rate may go above the baseline level for one day only with no change in the Basic Infertile Pattern (BIP). While the day of raised E1G excretion rate is unavailable for intercourse, the following day with a baseline excretion rate of E1G and a continued Basic Infertile mucus pattern indicates a return to the infertile phase.

If the E1G excretion rate remains above the level for 2 or more days then it is recommended that intercourse be suspended in anticipation for the mid-cycle fall in E1G excretion rate and that PdG measurements be commenced on this day. If the PdG excretion rate rises to the cut off, ovulation has occurred and the late infertile days have commenced and intercourse may be resumed. However, if the PdG excretion rate remains low two to three days after the E1G fall, testing of the E1G excretion rate is recommenced or continued. If the E1G excretion rate returns to baseline and remains at baseline for three days with an associated low PdG excretion rate, the infertile phase has returned. Intercourse can be resumed applying the E1G baseline in conjunction with the Basic Infertile Pattern.

In some cycles the PdG excretion rates may rise but do not reach cut off before bleeding begins. In other cycles the PdG rise to the cut off is slow. Intercourse can be resumed on the fourth day after the E1G peak, provided that a clear rise has been recorded in the PdG values and they have reached three quarters of the cut off value. This is known as the “three quarter cut off rule”. However, a contact person should be consulted before using this rule for the first time and if ever in doubt about its application.

When monitoring ovarian activity, stereotyped patterns are not expected and the hormone values should not be ignored no matter what the woman expects; they are very unlikely to be wrong.

### Exemplary Application

CB fully breast feeding for 4.5 months. The client commenced monitoring at home and a baseline was established. The advice on potential fertility was made by a trained Monitor technician based on the above guidelines.

TABLE 8
Use of the Ovarian Monitor for Return to Fertility
after Breastfeeding (Pregnancy Avoidance)

| | E1G | |
|---|---|---|
| Weeks Post Partum | nmol/24 hr (OM) | Decision |
| 19 | 155 | Safe 1 week |
| 20 | 144 | Safe 1 week |
| 21 | 110 | Safe 1 week |
| 22 | 150 | Safe 5 days |
| 23 | 132 | Safe 4 days |
| 24 | 110 | Safe 3 days |
| 25 | 160 | Safe 3 days |

### 4. Achievement of Pregnancy

An example of a successful conception using the Ovarian Monitor to measure the E1G and PdG excretion rates is given below.

Timing of intercourse to the day of the drop in E1G excretion rates can be an effective means of attaining pregnancy when difficulty has been experienced. This woman collected her urine samples and waited until the day of the drop in E1G excretion rate was identified before having intercourse. She conceived successfully in the second such cycle.

TABLE 9
Use of the Ovarian Monitor to Attain Pregnancy

| | E1G | PdG | |
|---|---|---|---|
| Cycle day | nmol/24 hr | μmol/24 hr | Intercourse |
| 7 | 150 | | |
| 8 | 170 | | |
| 9 | 170 | | |
| 10 | 220 | | |
| 11 | 245 | | |
| 12 | 340 | | |
| 13 | 400 | | |
| 14 | 280 | | Yes |
| 15 | 210 | 2.2 | |
| 16 | 180 | 2.0 | |
| 17 | | 5.6 | |
| 18 | | | |
| 19 | | | |
| 20 | | 18.4 | |

The E1G peak excretion day was day 13 and the only recorded act of intercourse occurred on day 14 (the day of the E1G excretion rate decline). The PdG excretion rates remained low for the next two days but then began to rise almost reaching the Monitor threshold value of 6.3 μmol/24 hr on day 17. A PdG value taken on day 20 confirmed that ovulation had occurred (biochemically) since the PdG excretion rate was >13 μmol/24 hr and that the luteal phase was adequate. A positive pregnancy test was obtained on day 33.

In general, the first treatment to be considered in a case of difficulty in achieving conception should be an analysis of the menstrual cycle for proof of fertile ovulation. Thus measurement of the E1G and PdG excretion rates for a complete cycle allows an assessment of whether the patient is ovulating, whether the luteal phase is adequate or whether it is short. All of these factors are a bar to conception. If the E1G excretion rate rises at an average of 140% per day to reach a mid-cycle peak and then the PdG excretion rate rises through 4-6 μmol/24 hr, to exceed 6.3 μmol/24 hr (luteinisation), then through 9 μmol/24 hr (ovulatory) and finally 13 μmol/24 hr (adequate luteal phase) with a luteal phase length of 12-16 days (normal luteal phase length), the cycle is a normal fertile ovulatory one. Timing of intercourse for three months using the day of the fall in E1G excretion rates (which is quite dramatic) is then a possible option before further clinical intervention and it may aid conception in the cases of male subfertility or nonendocrinological sources of female subfertility. If pregnancy does not occur within this time frame then further treatment should be sought. If any of the cycle parameters are abnormal then clinical assistance is advisable and monitoring is advantageous.

### 5. Gonadotrophin Therapy

The application of the E1G and PdG excretion rates to aid in the performance of gonadotrophin therapy using the incremental dosage procedure (Brown et al., J of Obstetrics & Gynecology of the British Commonwealth 76(4):289-307 (1969) is given here based on an MD thesis by Dr Simon Thornton (1990). In this procedure the gonadotrophin (HMG) is administered in a low dose which is increased incrementally until an appropriate response is elicited from the ovaries as shown by increasing E1G excretion rates.

### Billing Codes

In certain embodiments, a particular diagnosis is assigned a unique billing code, which can for example, allow the electronic transmission of a diagnosis to a patient, health care provider, or insurance company.

TABLE 10
Assignment of Billing Codes

Diagnosis CPT Billing code
Anovulation associated with Infertility -628.0
unexplained infertility-628.9
menopausal symptoms- 627.2
perimenopausal menorrhagia-627.0
postmenopausal bleeding-627.1
premature menopause-256.31
amenorrhea-626.0
hormone imbalance, unspecified-259.9
decreased libido-799.81
chronic fatigue-780.71
nervousness-799.2
osteoporosis-733.00
Premenstral syndrome-625.4
ovulation bleeding-626.5
Dysfunctional Uterine Bleeding.-626.8
hormone replacement therapy- V07.4
surgical menopause syndrome- 627.4
hypomenorrhea-626.1
hyperstimulated ovaries-614.8
polycystic ovarian disease-256.4
habitual aborter -currently pregnant again-646.33
missed abortion-632
threatened abortion-640.03

### Incremental Dosage Procedure

Choice of the initial does of HMG. In the patient's first cycle the starting dose should be 75 International units (IU) per day. If the patient has had a previous cycle then the dose chosen should be the same as that which previously resulted in satisfactory follicular development. If in the previous cycle over stimulation or hyperstimulation occurred and the cycle was cancelled, the dose selected should be lower than that previously resulting in over stimulation.

When to start injections. Patients with amenorrhoea and low gonadotrophin (GT) levels are unlikely to bleed in response to progesterone withdrawal and injections may therefore be started immediately after a baseline E1G value has been performed. In oligomenorrhoeic patients with endogenous GT activity, the treatment cycle should start within 2 weeks of a spontaneous or progesterone induced withdrawal bleed.

Baseline E1G excretion rate. A baseline E1G excretion rate test is carried out and if the value is low (<100 nmol/24 hours) then treatment may be commenced. If the E1G value is >100 nmol/24 hours, this may be due to either spontaneous ovarian activity or pregnancy. Pregnancy should therefore be excluded. If the patient has not had a recent period then one should be induced with a progesterone withdrawal bleed. If the patient is very obese, is not pregnant and had a recent period, then treatment may be started even if E1G values are >100 nmol/24 hours.

The follicular phase. Injections of HMG are commenced and given daily for 4-5 days. Daily E1G tests are carried out from the 5th day of HMG injections and continued daily until the HCG injection is given. Results are compared with the E1G baseline excretion rate from the previous week. If there has been no change the HMG dose is increased on the sixth day of HMG injections by a factor of approximately 1.3-1.5. This dose is continued for a further 4-5 days and daily E1G monitoring continued. If there is no response after 4-5 days to this increase in HMG, then the HMG dose is again increased and daily monitoring continued. Incremental dose steps of approximately 1.3-1.5 (75 IU, 112.5 IU, 150 IU, 225 IU, 300 IU) are continued at 4-5 day intervals until a response is noted. If the E1G excretion rate increases, but then “plateaus” before reaching 200 nmol/24 hr, the sample should be repeated to confirm the “plateauing”. If the E1G value has failed to rise on the following day then the HMG dose should be increased. When a response occurs, HMG is continued until the E1G values have reached 200 nmol/24 hr. An ultrasound scan is arranged for the following day. Ideally this should be a vaginal ultrasound scan, which gives superior quality follicular imaging compared to the original abdominal approach. In most cases it is possible to manage the cycle with a single ultrasound scan only. When the leading follicle size is <18-19 mm on the day of the ultrasound scan, it may be assumed that the leading follicle grows at approximately 2 mm a day and the appropriate day for giving HCG estimated accordingly. If the leading follicle is <14 mm a repeat scan in a further 48 hours is recommended to get a clear idea of the size and number of follicles present prior to HCG administration.

The ovulating HCG injection. The ovulating injection of HCG is given when the leading follicle reaches 18-19 mm. HCG injections are usually withheld if E1G excretion rates are rising excessively quickly (doubling or nearly doubling each day), if the E1G value is >750 nmol/24 hr or if there are more than 3 mature follicles of 18 mm or more present on the day HCG should ideally be given. The dose of HCG chosen is the minimum dose that results in ovulation for that patient. This is usually given 36 hrs after the final HMG dose. The usual starting dose is 3000 IU or 5000 IU. Intercourse is normally recommended on the night of HCG administration, the following night and every two nights in the early luteal phase.

The luteal phase, Day 0. On the day that HCG is given (=day 0), a PdG test is performed to see if premature luteinisation has occurred and also to establish a baseline for later changes in PdG in the luteal phase.

The luteal phase, Day +3. E1G and PdG tests are carried out. These tests give a good guide to the likely luteal phase pattern of E1G and PdG. If the E1G value has dropped (similar to the normal cycle pattern) hyperstimulation is unlikely in this cycle and the luteal phase support injection (HCG 1000 IU) can be given with confidence on day +6 if it falls on the weekend.

The luteal phase, Day +6. E1G and PdG tests are both done. If the E1G is >1000 nmol/24 hr or the patient is in pain, no luteal phase support injection is given. The PdG value should still be rising.

The luteal phase, Day +9 and Day +12. If holding injections have been given, the only test that is needed is a PdG excretion rate test on day +9 to confirm ovulation (PdG>12.2 μmol/24 hr). If ovulation is confirmed, holding injections are given on day +9 and day +12 and no further tests are required until a pregnancy test on day +22. If holding injections were not given, E1G and PdG excretion rate tests are performed on day +9 and day +12. If both are still high and rising, no luteal phase support injections are given. However, if there is a fall in E1G or PdG excretion rates and the patient is not in pain, one or two phase support injections on day +9 and day +12 are given.

The late luteal phase. PdG excretion rate tests on day +15, +18 and +21 may be done for the patient's curiosity. If the levels are still rising this is suggestive of a conceptual cycle. However they do not alter the patient's management and are of no particular value for prognosis.

If the patient has not had a period by day +22, a pregnancy test is performed. (By waiting until day +22 any exogenous HCG will be cleared from the body).

If the pregnancy test is positive, an early scan is arranged to check on foetal number, position and viability.

Second and succeeding cycles. When conception has not occurred during the first cycle, treatment is recommenced after the next period. The doses used in the second treatment cycle depend on responses obtained during the first, so the starting dose of HMG is that which gave a successful response in the first. Once a patient's HMG requirements have been determined they are usually reproducible from cycle to cycle. If there is any doubt however the next lower HMG dose is used and the dose increased incrementally as before. If ovulation does occur in the first cycle then the same dose of HCG is used in subsequent cycles. If ovulation does not occur then HCG doses are increased incrementally, in subsequent cycles (5000, 10,000, 20,000 IU etc.) until ovulation does occur.

Interpretation of home results. The use of the E1G and PdG excretion rate tests for home monitoring of GT ovulation induction is dependent on knowledge of normal cycle outcomes and also the possible problems that can occur with point-of-care monitoring.

Although different protocols can be written, it is clear that ready access to measurement of E1G and PdG excretion rates, as with the current tests, gives ready access to a therapy which is proven, safe and successful.

### 6. Application of PMP to Animal Fertility

All dairy farmers are interested in simple, cheap methods for pinpointing estrous for artificial insemination programmes. Although their preferred fluid is obviously milk we have made some progress in the measurement of urinary E1G and PdG for detection of estrus.

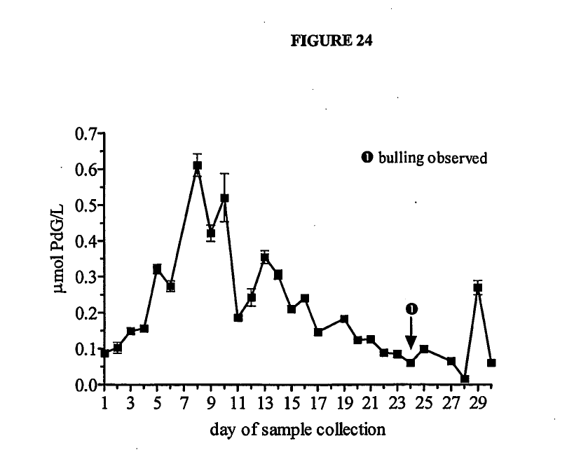

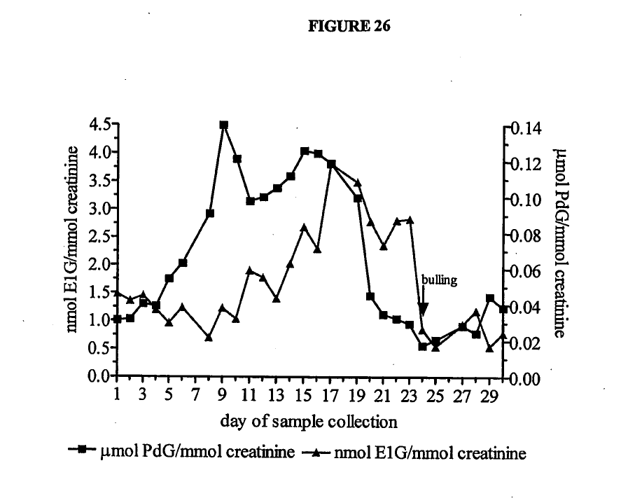

Like humans, cow hormone profiles benefit from some correction for urine production rate. FIG. 24 shows the PdG urinary profile obtained by ELISA without correction for urine volume, and FIG. 26 the same data after correction for urine volume by creatinine. Note how much the profile is modified by correcting the data for urine volume. After correction, the profile is converted into a very steep and broad peak. The day of observed bulling occurred on day 24 of the urine collection period.

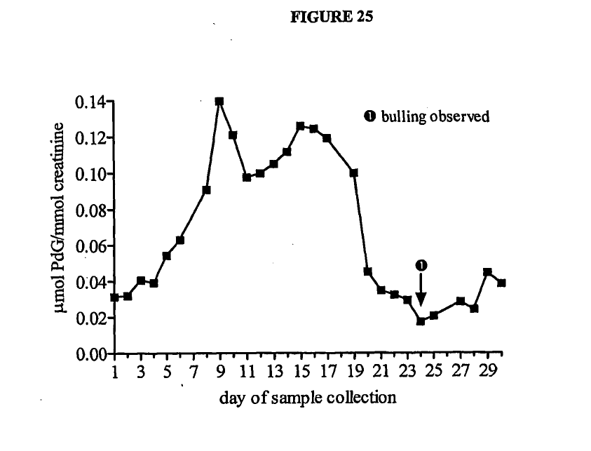

FIG. 25 shows the E1G urinary excretion data also corrected for creatinine excretion. The estrus cycle of a cow varies from a woman in that the progesterone production of luteal phase clearly overlaps with the follicular development and estrogen production of the next cycle. Ovulation occurs around the day of the E1G fall (similar to in humans). As this coincides with the fall of PdG levels from the previous luteal phase, a correction for the rate of urine production rate is not strictly necessary for the detection of estrus in the cow. Instead the E1G/PdG ratio can be used. As both creatinine concentrations for the E1G and PdG data are necessarily the same for each individual day, the units effectively cancel out and collection of data as mol/L becomes sufficient—see FIG. 26. FIG. 26 shows how if the E1G/PdG ratio is used, the day of estrus is easily predicted as the day of the big fall from a peak.

Although the standard curves obtained with the PMP cassettes are too sensitive to be used with the human menstrual cycle without dilution, the cow urine is excreted at significantly lower concentrations. Thus the standard curve obtained with the PMP cassettes may be appropriate for use with the undiluted cow urine. The day of lowest and highest PdG concentration (μmol/L) over the 30 days of collection was day 28 and 8 respectively (see FIG. 8). Because there is no correction for urine volume with the per litre units, the ratio between highest and lowest value is generally higher than that measured using a correction factor and the standard curve generally must cover a wider range so it can take into account the full range of possible values. For example it must be able to measure from a cow producing low levels of PdG and excreting high volumes of urine up to the levels of a cow producing high levels of PdG and excreting low volumes of urine. This is apparent here: when the data is corrected for creatinine (μmol PdG/mmol creatinine) the PdG ratio for the highest versus the lowest excretion rate is 5.3, but when the PdG ratio for the highest versus lowest for raw PdG concentration in the urine (μmol PdG/L) is used the ratio 36.9—i.e a standard curve to measure the uncorrected data (μmol PdG/L) requires a 7 fold greater range.

The two most extreme samples for PdG content were run directly on the PMP cassettes without dilution in triplicate, the values read off the standard curve and compared with the equivalent ELISA data that was also collected in triplicate. Day 8 gave a value of 0.61 μmol/L on the ELISA and 0.41 μmol/L on the PMP cassettes. Day 28 gave a value of 0.0165 μmol/L on the ELISA and 0.0177 μmol/L on the PMP cassettes.

Thus the agreement between the ELISA data and the PMP cassette data was exceptional at the extreme ranges of undiluted urinary PdG values obtained over a cow estrus cycle. The position of the standard curves obtained with the PMP cassettes are too sensitive to be able to be used with human samples without further dilution, but they appear initially at least, to be exceptionally well suited for the measurement PdG values likely to be encountered over a cow estrus cycle.

From the foregoing, it will be appreciated that, although specific embodiments of the invention have been described herein for the purpose of illustration, various modifications may be made without deviating from the spirit and scope of the invention. Accordingly, the present invention is not limited except as by the appended claims.

All patents, patent applications, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Additionally, all claims in this application, and all priority applications, including but not limited to original claims, are hereby incorporated in their entirety into, and form a part of, the written description of the invention. Applicants reserve the right to physically incorporate into this specification any and all materials and information from any such patents, applications, publications, scientific articles, web sites, electronically available information, and other referenced materials or documents. Applicants reserve the right to physically incorporate into any part of this document, including any part of the written description, the claims referred to above including but not limited to any original claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features reported and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for measuring fertility in a mammal, comprising:
obtaining a body fluid sample from a mammalian female subject;
contacting said sample with a solid phase capture element, said capture element comprising a first binding agent and a second binding agent, wherein said first binding agent is capable of binding an estrogen metabolite and said second binding agent is capable of binding a progesterone metabolite;
determining the excretion rate of said estrogen metabolite and said progesterone metabolite; and
determining the ovulation cycle status of said female subject based upon the relative excretion rates of said estrogen metabolite and said progesterone metabolite.

2. The method according to claim 1 wherein said estrogen metabolite is estrone glucuronide.

3. The method according to claim 1 wherein said progesterone metabolite is pregnanediol glucuronide.

4. The method according to claim 1 wherein said estrogen metabolite is estrone glucuronide and said progesterone metabolite is pregnanediol glucuronide.

5. The method according to claim 4 wherein said first binding element comprises an antibody capable of binding estrone glucuronide and said second binding element comprises an antibody capable of binding pregnanediol glucuronide.

6. The method according to claim 5 wherein one or both of estrone glucuronide and pregnanediol glucuronide are detected by binding to a polyclonal antibody.

7. The method according to claim 5 wherein one or both of estrone glucuronide and pregnanediol glucuronide are detected by binding to a monoclonal antibody or a binding fragment thereof.

8. The method according to claim 1 wherein said binding element is a single solid phase membrane or strip and both analytes are detected following binding to said strip.

9. The method according to claim 8 wherein said capture element is a single lateral flow strip.

10. The method according to claim 9 wherein said test strip contains antibodies or antibody fragments that bind estrone glucuronide and pregnanediol glucuronide.

11. The method according to any one of claims 2 to 10 wherein the antibodies against estrone glucuronide and pregnanediol glucuronide on the test strip are capable of being associated with a detection element.

12. The method according to any one of claims 2 to 10 wherein the antibodies against estrone glucuronide and pregnanediol glucuronide on the test strip are conjugated to a detection element.

13. The method according to claim 11 or 12 wherein said detection element is not colorimetric.

14. The method according to claim 11 or 12 wherein said detection element is a paramagnetic particle.

15. The method according to claim 1 wherein the mammal is a farm animal.

16. The method according to claim 1 wherein the mammal is bovine.

17. A method according to claim 16 comprising the step of comparing the excretion rates of said estrogen metabolite and said progesterone metabolite with a compilation of bovine estrogen metabolite and progesterone metabolite values determined over the course of at least one bovine ovulation cycle.

18. The method according to claim 17 wherein said compilation of bovine estrogen metabolite and progesterone metabolite values is an electronic database.

19. The method according to claim 1 wherein the mammal is equine.

20. The method according to claim 1 wherein the mammal is a human.

21. The method according to claim 1 wherein the body fluid is urine.

22. The method according to claim 21 further comprising determining the volume of said urine sample.

23. The method according to claim 21 wherein said body fluid is urine that is collected once.

24. The method according to claim 21 wherein said body fluid is urine that is collected without reference to a time interval.

25. The method according to claim 21 wherein said body fluid is urine that is collected over a specified time period.

26. The method according to claim 21 wherein said body fluid is urine that is collected without respect to a specified time period.

27. The method according to claim 21, wherein prior to quantifying the excretion rate of said estrogen metabolite and said progesterone metabolite, the volume of said urine sample is adjusted.

28. The method according to claim 1 wherein said body fluid is urine that is collected without respect to a specified time period and, before said excretion rates are determined, a sample volume adjustment step is performed by a sample dispensing device that makes a sample volume adjustment as needed.

29. The method according to claim 1 wherein said body fluid is urine that is collected without respect to a specified time period and a sample volume adjustment step is preformed according to an algorithm.

30. The method according to claim 1 wherein said body fluid is urine that is collected without respect to a specified time period and a sample volume adjustment step is preformed according to an algorithm based upon a spectroscopic analysis of the urine sample.

31. The method according to claim 1 wherein said body fluid is urine that is collected without respect to a specified time period and a sample volume adjustment step is preformed according to an algorithm based upon the specific gravity of the urine sample.

32. The method according to any one of claim 29, 30, or 31 wherein said algorithm is a computer algorithm.

33. The method according to claim 1 wherein said body fluid is urine that is collected over at least a 3 hour time period.

34. The method according to claim 1 wherein said body fluid is urine that is collected over at least a 3 hour time period and the volume is adjusted to a normalized volume.

35. The method according to claim 1 wherein said body fluid is urine that is collected over at least a 3 hour time period and the volume is adjusted to a normalized volume equal to about 150 ml/hr.

36. The method of claim 1 wherein the time frame for optimal fertility is determined.

37. The method of claim 1 wherein the day of ovulation is determined.

38. The method according to claim 1 further comprising the step of determining a time frame for optimal fertility for performing an in vitro fertilization of said female subject.

39. The method according to claim 32 wherein the step of adjusting said volume comprises normalizing the volume.

40. The method according to claim 1, further comprising the step of using an algorithm for correction of urinary volume bias before excretion rates are determined.

41. The method of claim 1 wherein a solid phase test strip with paramagnetic particles is used, wherein each said estrogen metabolite is detected by a first paramagnetic particle and said progesterone metabolite is detected by a second paramagnetic particle.

42. The method according to claim 1 wherein the amount of said estrogen metabolite and said progesterone metabolite is quantified.

43. The method according to claim 1 wherein a threshold amount of said estrogen metabolite and said progesterone metabolite are detected as a positive or negative value.

44. The method according to claim 1 that is used to determine a period of maximal fertility within a menstrual cycle.

45. The method according to claim 1 wherein a sample is taken daily and the excretion rates of said estrogen metabolite and said progesterone metabolite are quantified daily for a set interval of time.

46. The method according to claim 1 comprising the step of using a portable detector to measure excretion rate.

47. The method according to claim 22 wherein said portable detector is in communication with database comprising historical values of excretion rates for progesterone or a progesterone metabolite.

48. The method according to claim 22 wherein said portable detector is in communication with database comprising historical values of excretion rates for estrogen or a estrogen metabolite.

49. A test strip for performing the method of claim 1.

50. A test strip comprising a first binding element capable of binding a estrogen metabolite and a second binding element capable of binding a progesterone metabolite.

51. The test strip according to claim 42 wherein said first binding element and said second binding element are antibodies or fragments thereof.

52. A quantitative test strip for detecting and quantifying estrone glucuronide and pregnanediol glucuronide.

53. A kit for performing the method of claim 1, said kit comprising a container for a solid phase capture element and instructions for use of the kit, said capture element comprising a first binding agent and a second binding agent, wherein said first binding agent is capable of binding an estrogen metabolite and said second binding agent is capable of binding a progesterone metabolite.

54. A reader for performing the method of claim 1, said reader comprising:
a holder for a solid phase capture element;
a detection element for detecting a photometric or electroactive signal; and
means for transmitting and/or analyzing said photometric or electroactive signal.

55. A method for measuring fertility in a mammal, comprising:
obtaining a body fluid sample from a mammalian female subject;
contacting said sample with a first binding agent and a second binding agent, wherein said first binding agent is capable of binding an estrogen metabolite and said second binding agent is capable of binding a progesterone metabolite;
determining the excretion rate of said estrogen metabolite and said progesterone metabolite; and
determining the ovulation cycle status of said female subject based upon the relative excretion rates of said estrogen metabolite and said progesterone metabolite.

56. A fertility monitor comprising:
a sample dispenser;
a sensor for detecting the presence of at least two analytes in the sample;
a processor for calculating;
communication means to central database or internal data storage comprising estrone glucuronide and pregnanediol glucuronide excretion rate data.

57. A computer program for home users for providing information on the use of the method of claim 1 and to permit display of the data on a periodic basis.

58. A computer program product for the display and analysis of urinary estrone glucuronide and pregnanediol glucuronide excretion rates suitable for use with a computer in communication with an electronic database of menstrual cycle comprising historical estrone glucuronide and pregnanediol glucuronide excretion rate values.

59. A method for measuring fertility in an mammal, said method comprising the steps of
obtaining a body fluid sample from a female subject;
contacting said sample with a capture element comprising a binding agent capable of binding a progesterone metabolite;
quantifying the excretion rate of said progesterone metabolite;
determining the ovulation cycle status of said female subject based upon the excretion rate of the progesterone metabolite.

60. A method for measuring fertility in an mammal, said method comprising the steps of:
obtaining a body fluid sample from a female subject;
contacting said sample with a capture element capable of binding an estrogen metabolite;
quantifying the excretion rate of said estrogen metabolite;
comparing the excretion rate of said estrogen metabolite against an estrogen metabolite excretion rate made within the same ovarian cycle; and
determining the ovulation cycle status of said female.

61. A method for measuring fertility in an non-human mammal, said method comprising the steps of
obtaining a body fluid sample from a non-human female subject;
contacting said sample with a capture element, said capture element comprising a first binding agent and a second binding agent, wherein said first binding agent is capable of binding an estrogen metabolite and said second binding agent is capable of binding a progesterone metabolite;
quantifying the excretion rates for said estrogen metabolite and said progesterone metabolite;
determining the ovulation cycle status of said female subject based on the relative excretion rates of said estrogen metabolite and said progesterone metabolite.

62. A method of monitoring the physiologic status of one or more remotely located subjects wherein a central data processing system is configured to communicate with and receive data from one or more subject monitoring systems, wherein each subject monitoring system is capable of one or more of receiving, storing, and analyzing subject data, the method-comprising the steps of:
obtaining a sample from a subject for analysis;
contacting the sample with an analyte detector associated with a subject monitoring system;
measuring a photometric or electroactive signal corresponding to an analyte on the detection device and detecting one or more analyte;
performing an exchange of data between said subject monitoring system and said central data processing system;
generating a computer program product output comprising historical or real time physiologic status assessment data of said subject, wherein said computer program product output is in communication with the central data processing system;
analyzing said subject data from one or more subject monitoring systems;
determining the status of the subject based on the analysis performed by said computer program; and
communicating, transmitting, or displaying the identified subject status and a therapeutic management recommendation for one or more subjects.

63. A method of monitoring physiologic status of one or more remotely located subjects in need of therapeutic management wherein a central data processing system is configured to communicate with and receive data from a one or more subject monitoring systems, wherein each subject monitoring system is capable of one or more of receiving, storing, and analyzing subject data, the method-comprising the steps of:
obtaining a sample from a subject for analysis;
contacting the sample with an analyte detector associated with a subject monitoring system;
measuring a photometric or electroactive signal corresponding to an analyte on the detection device and detecting one or more analyte;
performing a volumetric correction for fluid volume bias using a computer executable algorithm;
quantifying the excretion rate of one or more analyte;
transmitting information from a subject monitoring system to a central data processing system;
generating a computer program product output comprising historical and/or real time physiologic status assessment data of said subject, wherein said computer program product output is in communication with the central data processing system;
analyzing said subject data from one or more subject monitoring systems;
optimizing accuracy of fertility status assessment and or fertility status prediction of individual fertility endpoints by statistical comparison by individual historical data and or subject population historical data;
determining the status of the subject based on the analysis performed by said computer program; and
communicating, transmitting, and/or displaying the identified subject status and a therapeutic management recommendation for one or more subjects via at least one remotely located client in communication with said central data processing system and/or subject monitor system.

64. A method of monitoring fertility status of a plurality of remotely located female subjects in need of fertility management wherein a central data processing system is configured to communicate with and receive data from a plurality of respective subject monitoring systems, wherein each subject monitoring system is capable of receiving, storing, and analyzing subject data, the method-comprising the steps of:
identifying a detection device associated with a subject;
verifying the identity of a detection device;
verifying the identity of a subject monitoring system associated with a subject;
obtaining a body fluid sample from said subject for analysis;
capturing said sample on detection device suitable for detection on the subject monitoring system;
assessing the detection device and measuring the signals from the analyte on the detection device to provide inputs to computer program product A (Algorithm A); and/or computer program product B (Algorithm B);
analyzing the obtained subject data transmitted from a subject monitoring system substantially simultaneously with the transmission thereof to the subject computer CPU and/or central data processing system to determine the subject's fertility status based on the analysis performed by said computer program product (Algorithm A);
generating a computer program product output A (database A) and/or computer program product output B (database B) comprising the fertility status assessment data from said computer program product A and/or computer program product B;
updating the computer program product output A (database A) and/or computer program product output B (database B) with additional fertility status assessment data inputs generated from detection and analysis of additional fertility status assessment data from the subject;
obtaining subject data from a plurality of subject monitoring systems at the central data processing system, wherein the subject data comprises fertility status assessment data;
analyzing said subject data transmitted from a plurality of subject monitoring systems at the central data processing system substantially simultaneously with the transmission thereof to the computer of a physician or designated health care professional;
determining the subject's fertility status based on the analysis performed by said computer program product B (Algorithm B) and to identify fertility issues of individual Subjects including potential abnormalities when compared against fertility status assessment data from broader subject populations;
communicating, transmitting, and/or displaying the identified subject fertility status and fertility management recommendation for each respective subject via at least one remotely located client in communication with said central data processing system and/or respective subject monitor system; and
transmitting information pertaining to the fertility status and fertility issues of individual Subjects including potential abnormalities when compared against fertility status assessment data from broader subject populations.

65. A method of monitoring physiologic status of one or more remotely located subjects in need of therapeutic management, wherein a central data processing system is configured to communicate with and receive data from one or more subject monitoring systems, wherein each subject monitoring system is capable of receiving, storing, and analyzing subject data, the method-comprising the steps of:
obtaining a sample from said subject for analysis;
capturing said sample on detection device suitable for detection on the subject monitoring system;
assessing the detection device and measuring the signals from the analyte on the detection device;
analyzing the obtained subject data transmitted from a subject monitoring system substantially simultaneously with the transmission thereof to the central data processing system to determine the subject's clinical or physiologic status;
generating a computer program product output (database) comprising historical and real time physiologic status assessment data of said subject or subjects in communication with the central data processing system;
analyzing said subject data transmitted from one or a plurality of subject monitoring systems at the central data processing system substantially simultaneously with the transmission thereof to the computer of a physician or designated health care professional;
determining the subject's clinical or physiologic status based on the analysis performed by said computer program and to identify clinical or physiologic issues of individual subjects including potential abnormalities when compared against clinical or physiologic status assessment data from broader subject populations; and
communicating, transmitting, and/or displaying the identified subject physiologic status and therapeutic management recommendation, including potential abnormalities when compared against clinical or physiologic status assessment data from broader subject populations, via at least one remotely located client in communication with said central data processing system and/or respective subject monitor system.

66. The method according to any one of claims 62-65, wherein the subject sample is selected from crevicular fluid, sweat, sebum, vaginal fluid, whole blood, serum, plasma, cerebrospinal fluid, urine, lymph fluids, external secretions of the respiratory, intestinal and genitourinary tracts, tears, saliva, milk, or white blood cells.

67. The method according to claim 69, wherein sample comprises urine.

68. The method according any one of claims 62-65, wherein said detection devices comprises a solid phase capture element selected from porous materials, glass fiber, membranes, papers, strips, pads, nylon, nitrocellulose, or polyester materials.

69. The method according to claim 68, wherein said analyte detection device comprises a lateral flow suitable for detection of one or more metabolites.

70. The method according to claim 68, wherein said detection device comprises a paramagnetic particle embedded assay detection pad.

71. The method according to claim 68, wherein said analyte generates photometric or electroactive detection signals.

72. The method according to claim 68, wherein said electroactive analytes are conjugated to paramagnetic particles.

73. The method according to claim 68, wherein said analytes is selected from hormones or hormone metabolites.

74. The method according to claim 73, wherein said analyte is selected from the group comprising estrogen, progesterone, testosterone or metabolites thereof.

75. The method according to claim 74, wherein said analyte is urinary hormone metabolites.

76. The method according to claim 75, wherein said urinary metabolite is selected from the group comprising estrone 3-sulfate, 2-hydroxyestrone, 4-hydroxyestrone, 2-methoxyestrone, 4-methoxyestrone, 2-methoxyestrone 3-sulfate, 2-methoxyestrone 3-glucuronide, 16 alpha-hydroxyestrone, estradiol-17α, estradiol 17β, 16-glucuronide-estriol; estradiol-17beta 3-glucuronide; estradiol-17beta 3-sulfate, 2-hydroxy-estradiol-17β, 2-methoxy-estradiol-17β, 2-methoxyestradiol-17beta 3-sulfate, 2-methoxy-estradiol-17beta 3-glucuronide, 6β-hydroxy-estradiol-17β, 2-methoxyestradiol, 17-epiestriol, 2-hydroxyestradiol, 16-ketoestradiol, 16β-hydroestrone, 16-epiestriol.

77. The method according to claim 76, wherein said estrogen or metabolite thereof is selected from the group comprising estradiol, estrone, estriol, 2(OH) Estrone, 4 hydroxy-estrone, 16α-hydroxy-estrone, 2-methoxyestrone, and 4-methoxyestrone.

78. The method according to claim 77, wherein said estrogen metabolite is estrone glucuronide (E1G).

79. The method according to claim 76, wherein said progesterone or progesterone metabolite is selected from the group comprising 5β-pregnan-3α,20α-diol glucuronide, 5β-pregnan-3α-ol-20-1-(5β-pregnenolone) and 5α-pregnan-3α-ol-20-1-(5α-pregnenolone).

80. The method according to claim 79, wherein said progesterone metabolite is pregnanediol glucuronide (PdG).

81. The method according to claims 62-65, wherein said analysis and or assessment is performed by a computer executable algorithm.

82. The method according to claim 81, wherein said database comprises historical and real time physiologic status assessment data of said subject or subjects in communication with the central data processing system.

83. The method according to claim 82, wherein said database comprises historical and real time fertility status assessment data of said subject or subjects in communication with the central data processing system.

84. The method according to claim 83, wherein said database comprises historical and real time urinary metabolite excretion rates status assessment data of said subject or subjects in communication with the central data processing system.

85. The method according to claim 84, wherein said database comprises historical and real time urinary glucuronide excretion rates status assessment data of said subject or subjects in communication with the central data processing system.

86. The method according to claim 81, wherein said subject's clinical or physiologic status are determined based on comparison against clinical or physiologic status assessment data from broader subject populations and or said individual.

87. The method according to claim 81, wherein said communication is performed by a device selected from the group comprising a transmitter, a beeper, a receiver, a telephone, a modem, a cellular phone, a cable, an internet connection, a world wide web link, a television, a closed circuit monitor, a computer, a display screen, a telephone answering machine, facsimile machine, or a printer.

88. The method according to claim 81, wherein said database consists of data selected from the group consisting of physiologic data and behavioral data.

89. The method according to claim 90, wherein said data is selected from the group consisting of urinary metabolite data; blood glucose measurement; body temperature measurement; assessments data related to diet, exercise, stress, and the presence of illness.

90. The method according to claim 81, wherein said algorithm optimizes efficacy of the specific fertility regimen based on particular subject's reproductive condition;

91. The method according to claim 89, wherein said algorithm is configured to make automatic adjustments to a subject's self-monitoring and fertility management regimen based on subject-entered data.

92. The method according to claim 89, wherein said algorithm contain a database useful for evaluation of the effects of concurrent therapy for other non-fertility indication which might affect the fertility or ovulation cycle of the subject.

93. The method according to claim 81, wherein said SMS suitable for monitoring fertility management data of subjects is capable of detecting paramagnetic analyte signals.

94. The method according to claim 81, wherein said database contains data directed to fertility status-associated values, health status, diet, exercise, and medications taken; date and time information of the last measurement; and prescribed course of action regimen.

95. The method according to claim 81, wherein said algorithm calculates adjustments for a subject's ovulation variation according to a physician or health care professionals prescription as applied to the data entered into the SMS by the subject.

96. The method according to claim 81, wherein said fertility algorithm for use within a SMS include fertility management algorithm allows a physician or other health care professional to specify retrospective and/or supplemental adjustment regimens.

97. The method according to claim 81, wherein said database of medication interaction information is configured to allow a subject to query the database for information related to the subject's use of multiple medications.

98. The method according to claim 81, wherein said database of medication interaction information is configured to allow a subject to query the database for specific historical fertility data profile for each subject and/or historical fertility profiles for populations of subjects.

99. A method for diagnosing and treating a post partum condition in a female, comprising:
obtaining a body fluid sample from a mammalian female subject;
contacting said sample with a solid phase capture element, said capture element comprising a binding agent capable of binding estrogen or an estrogen metabolite;
quantifying said estrogen or estrogen metabolite; and
diagnosing a post partum condition in said female subject based upon the amount of said estrogen or estrogen metabolite; and
treating said post partum condition based upon the upon the amount of said estrogen or estrogen metabolite detected.

100. The method according to claim 99 comprising administering a hormone replacement based upon the amount of said estrogen or estrogen metabolite.

101. A method for treating menopause and/or symptoms associated with menopause in a female, comprising:
obtaining a body fluid sample from a mammalian female subject;
contacting said sample with a solid phase capture element, said capture element comprising a binding agent capable of binding an estrogen or an estrogen metabolite;
quantifying said estrogen or estrogen metabolite; and
detecting a post partum condition in said female subject based upon the amount of said estrogen or estrogen metabolite detected; and
treating menopause and/or symptoms associated with menopause based upon the amount of said estrogen or estrogen metabolite detected.

102. A method of claim 101 wherein said menopause is characterized as one of natural menopause, perimenopause, induced menopause, premature menopause, or post menopause.

103. A method of detecting cancer in a mammal, comprising:
obtaining a body fluid sample from a mammalian subject;
contacting said sample with a solid phase capture element, said capture element comprising a binding agent capable of binding an hormone or hormone metabolite;
determining the amount or excretion rate of said metabolite; and
correlating the amount or excretion rate of said hormone or hormone metabolite with the probability of said mammalian subject having cancer; and
recommending a treatment protocol for said patient based upon the upon the amount or excretion rate of said hormone or hormone metabolite.

104. The method of detecting cancer of claim 103, wherein said subject is a female suspected of having breast cancer, wherein said hormone or hormone metabolite is estrogen or a estrogen metabolite.

105. A method detecting a reproductive disorder in a mammalian female, comprising:
obtaining a body fluid sample from a mammalian subject;
contacting said sample with a solid phase capture element, said capture element comprising a binding agent capable of binding a hormone or hormone metabolite;
determining the amount or excretion rate of said hormone or hormone metabolife; and
correlating the amount or excretion rate of said hormone or hormone metabolife with the probability of said mammalian subject having one or more disorder selected from anovulation associated with infertility, unexplained infertility, perimenopausal menorrhagia, postmenopausal bleeding, premature menopause, amenorrhea, a hormone imbalance, decreased libido, chronic fatigue, nervousness, osteoporosis, premenstral syndrome, ovulation bleeding, dysfunctional uterine bleeding, hormone replacement therapy, surgical menopause syndrome, hypomenorrhea, hyperstimulated ovaries, polycystic ovarian disease, habitual aborter, missed abortion, and threatened abortion; and
recommending a treatment protocol for said patient based upon the upon the amount or excretion rate of said hormone or hormone metabolife.
